(19) United States
(12) Patent Application Publication
(10) Pub. No.: US 2006/0107352 A1
(43) Pub. Date: May 18, 2006

(54) Transgenic expression cassettes for expression of nucleic acids in the plant blooms

(75) Inventors: Christel Renate Schopfer, Quedlinburg (DE); Matt Sauer, Quedlingubrg (DE); Martin Klebsattel, Quedlinburg (DE); Ralf Flachmann, Quedlinburg (DE)

Correspondence Address: CONNOLLY BOVE LODGE & HUTZ, LLP, P O BOX 2207, WILMINGTON, DE, 19899, US

(73) Assignee: SunGene GmbH & Co. KGaA, Gatersleben (DE)

(21) Appl. No.: 10/524,648

(22) PCT Filed: Jul. 30, 2003
(86) PCT No.: PCT/EP03/08394

§ 371 Date: Sep. 14, 2005

(30) Foreign Application Priority Data
Aug. 20, 2002 (DE) 102 38 979.9
Oct. 11, 2002 (DE) 102 47 599.7

(51) Int. Cl. A01H 1/00; C12N 5/04; C12N 15/82

(52) U.S. Cl. 800/284; 435/468; 435/419

(57) ABSTRACT

The invention relates to methods for the targeted transgenic expression of nucleic acid sequences in the flower of plants, and to transgenic expression cassettes and expression vectors which comprise promoters having an expression specificity for the flower of plants. The invention further relates to organisms (preferably plants) transformed with these transgenic expression cassettes or expression vectors, to cultures, parts or propagation material derived therefrom, and to the use of the same for producing human and animal foods, seeds, pharmaceuticals or fine chemicals.

The invention relates to methods for the targeted transgenic expression of nucleic acid sequences in the flower of plants, and to transgenic expression cassettes and expression vectors which comprise promoters having an expression specificity for the flower of plants. The invention further relates to organisms (preferably plants) transformed with these transgenic expression cassettes or expression vectors, to cultures, parts or propagation material derived therefrom, and to the use of the same for producing human and animal foods, seeds, pharmaceuticals or fine chemicals.

The aim of biotechnological operations on plants is to produce plants with advantageous novel properties, for example for increasing the agricultural productivity, for increasing the quality of human foods or for producing particular chemicals or pharmaceuticals (Dunwell J M (2000) J Exp Bot 51 Spec No:487-96). A basic precondition for transgenic expression of particular genes is the provision of promoters which are functional in plants. Promoters are important tools in plant biotechnology for controlling the expression of particular genes in a transgenic plant and thus achieving particular traits of the plant.

Various promoters functional in plants are known, for example constitutive promoters such as the promoter of the agrobacterium nopaline synthase, the TR double promoter or the promoter of the cauliflower mosaic virus (CaMV) 35S transcript (Odell et al.(1985) Nature 313:810-812). A disadvantage of these promoters is that they are constitutively active in virtually all tissues of the plant. Targeted expression of genes in particular plant parts or at particular times of development is not possible with these promoters. There is thus a particularly great need for promoters having a defined activity profile and a specificity for particular plant tissues.

Promoters having specificities for various plant tissues such as anthers, ovaries, flowers, leaves, stalks, roots, tubers or seeds have been described. The stringency of the specificity and the expression activity of these promoters varies widely.

The flower of plants serves for sexual reproduction of flowering plants. The flowers of plants—especially the petals—frequently accumulate large amounts of secondary plant products such as, for example, terpenes, anthocyans, carotenoids, alkaloids and phenylpropanoids, which serve as scents, defensive substances or as colorants in the flower. Many of these substances are of commercial interest. In addition, the flower bud and the flower of the plant is a sensitive organ, especially to stress factors such as cold.

Flower-specific promoters such as, for example, the phytoene synthase promoter (WO 92/16635), the promoter of the P-rr gene (WO 98/22593) or the promoter of the APETALA3 gene (Hill T A et al. (1998) Development 125:1711-1721) are known. However, all these promoters have one or more disadvantages which are predjudicial to wide use:
1) within the flower they are specific for one or more flower tissues and do not guarantee expression in all tissues of the flower.
2) they are—as in the example of the APETALA3 gene which is involved in flower development—highly regulated during flower development and are not active in all phases of flower development.
3) they occassionally show strong secondary activities in other plant tissues.

Despite the large number of known plant promoters, there is a need for promoters having a specificity for the flower of plants and guaranteeing high expression over a long period of flower development and flowering.

It is an object of the present invention to provide methods and suitable promoters for the targeted transgenic expression of nucleic acids in flower tissues.

We have found that this object is achieved by providing promoters of ε-cyclase. These promoters show an usually strong expression in numerous flower organs.

A first aspect of the invention relates to methods for the targeted transgenic expression of nucleic acid sequences in the flower of plants, including the following steps
I. introduction of a transgenic expression cassette into plant cells, where the transgenic expression cassette comprises at least the following elements
   a) at least one promoter sequence of a gene coding for an ε-cyclase, and
   b) at least one further nucleic acid sequence, and
   c) where appropriate further genetic control elements,
where at least one of said promoter sequences and one further nucleic acid sequence are functionally linked together, and the further nucleic acid sequence is heterologous in relation to the promoter sequence or the plant cell, and
II. selection of transgenic cells which comprise said expression cassette stably integrated into the genome, and
III. regeneration of complete plants from said transgenic cells, where at least one of the further nucleic acid sequences is expressed in the flower.

A further aspect relates to transgenic expression cassettes as can be employed in the method of the invention. The transgenic expression cassettes preferably include for the targeted transgenic expression of nucleic acid sequences in the flower of plants
a) at least one promoter sequence of gene coding for an ε-cyclase, and
b) at least one further nucleic acid sequence, and
c) where appropriate further genetic control elements,
   where at least one promoter sequence and one further nucleic acid sequence are functionally linked together, and the further nucleic acid sequence is heterologous in relation to the promoter sequence.

In a preferred embodiment of the method of the invention and/or of the expression cassettes of the invention, “promoter sequence of a gene coding for an ε-cyclase” means a sequence selected from the group of sequences consisting of
i) the promoter sequence of the ε-cyclase from Tagetes erecta as shown in SEQ ID NO: 1, the ε-cyclase from Arabidopsis thaliana as shown in SEQ ID NO: 7, the ε-cyclase from Oryza sativa as shown in SEQ ID NO: 8, and
ii) functional equivalents of the promoter sequences as shown in SEQ ID NO: 1, 7 or 8 having substantially the same promoter activity as the promoter of the ε-cyclases as shown in SEQ ID NO: 1, 7 or 8 and
iii) functionally equivalent fragments of the sequences under i) or ii) having substantially the same promoter activity as the promoter of ε-cyclases as shown in SEQ ID NO: 1, 7 or 8.

It is particularly preferred for “promoter sequence of a gene coding for an ε-cyclase” to mean the promoter sequence from Tagetes erecta as shown in SEQ ID NO: 1 and functionally equivalent fragments thereof.

The expression cassettes of the invention may comprise further genetic control sequences and/or additional functional elements.

It is possible and preferred for the transgenic expression cassettes to make possible, through the nucleic acid sequence to be expressed transgenically, the expression of a protein encoded by said nucleic acid sequence and/or the expression of a sense-RNA, antisense-RNA or double-stranded RNA encoded by said nucleic acid sequence.

A further aspect of the invention relates to transgenic expression vectors which comprise one of the expression cassettes of the invention.

A further aspect of the invention relates to transgenic organisms which comprise one of the expression cassettes or expression vectors of the invention. The organism can be selected from the group consisting of bacteria, yeasts, fungi, nonhuman animals and plant organisms or of cells, cell cultures, parts, tissues, organs or propagation material derived therefrom, and the organism is preferably selected from the group of agricultural crop plants.

A further aspect of the invention therefore relates to an isolated nucleic acid sequence including the promoter of the ε-cyclase from Tagetes erecta as shown in SEQ ID NO: 1, and functionally equivalent fragments thereof.

In a preferred embodiment, the nucleic acid sequence of the invention or the transgenic expression cassette of the invention in the form of a functionally equivalent promoter sequence includes, besides the sequence shown in SEQ ID NO: 1, additionally the sequence coding for the 5′-untranslated region of the ε-cyclase gene from Tagetes erecta. The sequence described by SEQ ID NO: 2 is particularly preferred.

In a further preferred embodiment, the nucleic acid sequence of the invention or the transgenic expression cassette of the invention in the form of a functionally equivalent promoter sequence includes, besides the sequence shown in SEQ ID NO: 1, additionally the sequence coding for the 5′-untranslated region of the ε-cyclase gene from Tagetes erecta and a sequence coding for a transit peptide, preferably for the transit peptide of the ε-cyclase protein from Tagetes erecta as shown in SEQ ID NO: 4. This sequence is preferably oriented in the 3′ direction in relation to one of the promoters of the invention. The sequence described by SEQ ID NO: 3 is particularly preferred as promoter sequence in this connection.

A further aspect relates to the use of the isolated nucleic acid sequences, transgenic expression vectors or transgenic organisms of the invention for the transgenic expression of nucleic acids and/or proteins.

A further aspect of the invention relates to the use of the nucleic acid sequence of the invention for reducing the expression of an ε-cyclase. Included within this according to the invention are expression cassettes able to express a double-stranded RNA corresponding to the promoter sequence.

It is particularly preferred to use said transgenic organisms or cells, cell cultures, parts, tissues, organs or propagation material derived therefrom to produce human and animal foods, seeds, pharmaceuticals or fine chemicals, where the fine chemicals are preferably enzymes, vitamins, amino acids, sugars, saturated or unsaturated fatty acids, natural or synthetic flavorings, aromatizing substances or colorants. The invention further includes methods for producing said human and animal foods, seeds, pharmaceuticals or fine chemicals employing the trangenic organisms of the invention or cells, cell cultures, parts, tissues, organs or propagation material derived therefrom.

The transgenic expression cassettes of the invention are particularly advantageous for the following reason:
a) they impart selective expression in the flower of plant and make numerous applications possible, such as, for example, resistance to stress factors such as cold or targeted synthesis of secondary plant products. Expression takes place throughout the period of flower development with high activity.

“Expression” means the transcription of the nucleic acid sequence which is to be expressed transgenically, but may also include—in the case of an open reading frame in the sense orientation—the translation of the transcribed RNA of the nucleic acid sequence to be expressed transgenically into a corresponding polypeptide.

“Transgenic” means—for example in relation to a transgenic expression cassette, a transgenic expression vector, a transgenic organism or methods for the transgenic expression of nucleic acids—all constructions resulting from methods of genetic manipulation, or methods using such, in which either
a) an ε-cyclase promoter (e.g. as shown in SEQ ID NO: 1, 7 or 8) or a functional equivalent thereof or a functionally equivalent fragment of the aforementioned, or
b) the nucleic acid sequence which is to be transgenically expressed, are functionally linked to a promoter according to a), or
c) (a) and (b)
   are not located in their natural genetic environment or have been modified by methods of genetic manipulation, where the modifications may be for example substitutions, additions, deletions, inversions or inserts of one or more nucleotide residues. The promoter sequence of the invention (e.g. the sequence as shown in SEQ ID NO: 1, 7 or 8) contained in the expression cassettes is preferably heterologous in relation to the further nucleic acid sequence which is to be expressed transgenically and is functionally linked thereto. “Heterologous” means in this connection that the further nucleic acid sequence does not code for the gene which is naturally under the control of said promoter.

“Natural genetic environment” means the natural chromosomal locus in the original organism or the presence in a genomic library. In the case of a genomic library, the natural genetic environment of the nucleic acid sequence is preferably still retained at least in part. The environment flanks the nucleic acid sequence at least on one side and has a sequence length of at least 50 bp, preferably at least 500 bp, particularly preferably at least 1000 bp, very particularly preferably at least 5000 bp. A naturally occurring expression constant—for example the naturally occurring combination of the promoter as shown in SEQ ID NO: 1 and of a gene coding for a protein as shown in SEQ ID NO: 10 or 12—becomes a trangenic expression construct when the latter is modified by unnatural, synthetic (“artificial”) methods such as, for example, an in vitro mutagenesis. Appropriate methods are described (U.S. Pat. No. 5,565,350; WO 00/15815; see also above).

“Transgenic” means in relation to an expression (“transgenic expression”) preferably all expressions caused by use of a transgenic expression cassette, transgenic expression vector or transgenic organism—complying with the definitions given above.

The transgenic expression cassettes of the invention, and the transgenic expression vectors and transgenic organisms derived therefrom may include functional equivalents to the ε-cyclase promoter sequence described in SEQ ID NO: 1, 7 or 8.

Functional equivalents also include all the sequences derived from the complementary strand of the sequences defined by SEQ ID NO: 1, 7 or 8 and having substantially the same promoter activity. Particularly preferably included are the sequences shown in SEQ ID NO: 2 or 3, which, besides the promoter sequence, comprise the 5′-untranslated region or the 5′-untranslated region and the region coding for the transit peptide of the ε-cyclase from Tagetes erecta.

Functional equivalents means in particular natural or artificial mutations of the ε-cyclase promoter sequence described in SEQ ID NO: 1, 7 or 8, and the homologs thereof from other plant genera and species which still have substantially the same promoter activity as the ε-cyclase promoter as shown in SEQ ID NO: 1, 7 or 8.

A promoter activity is referred to as substantially the same when the transcription of a particular gene to be expressed is, under the control of, for example, a functional equivalent of the ε-cyclase promoter sequence described by SEQ ID NO: 1, 7 or 8, or of a functionally equivalent fragment thereof—under conditions which are otherwise unchanged—higher in at least one flower tissue than in another non-flower tissue, for example the root or the leaves. In this connection, the expression under the control of one of the promoters of the invention in a flower tissue is preferably at least twice or five times, very particularly preferably at least ten times or fifty times, most preferably at least hundred times, that in another non-flower tissue, for example the root or the leaves.

“Flower” generally means a shoot of limited growth whose leaves have been transformed into reproductive organs. The flower consists of various “flower tissues” such as, for example, the sepals, the petals, the stamens or the carpels. Androecium is the term used for the totality of stamens in the flower. The stamens are located within the circle of petals and sepals. A stamen is composed of a filament and of an anther located at the end. The latter in turn is divided into two thecae which are connected together by a connective. Each theca consists of two pollen sacs in which the pollen is formed.

“Targeted” means in relation to expression in the flowers of plants preferably that the expression under the control of one of the promoters of the invention in at least one plant flower tissue is at least ten times, particularly preferably at least fifty times, very particularly preferably at least one hundred times that in a non-flower tissue such as, for example, the leaves.

The sequences preferably employed for estimating the level of expression are those which code for easily quantifiable proteins. Very particular preference is given in this connection to reporter proteins (Schenborn E, Groskreutz D. (1999) Mol Biotechnol 13(1): 29-44) such as the green fluorescent protein (GFP) (Chiu W L et al. (1996) Curr Biol 6:325-330; Leffel S M et al. (1997) Biotechniques 23(5):912-8), chloramphenicol acetyltransferase, luciferase (Millar et al. (1992) Plant Mol Biol Rep 10:324-414), β-glucuronidase or β-galactosidase. Very particular preference is given to β-glucuronidase (Jefferson et al. (1987) EMBO J 6:3901-3907).

“Conditions which are otherwise unchanged” means that the expression initiated by one of the transgenic expression cassettes to be compared is not modified by combination with additional genetic control sequences, for example enhancer sequences. Unchanged conditions further means that all general conditions such as, for example, plant species, stage of development of the plants, culture conditions, assay conditions (such as buffer, temperature, substrates etc.) are kept identical between the expressions to be compared.

Functional equivalents of the ε-cyclase promoter as shown in SEQ ID NO: 1, 7 or 8 preferably includes sequences which
a) have substantially the same promoter activity as the ε-cyclase promoter as shown in SEQ ID NO: 1, 7 or 8, and
b) have a homology of at least 50%, preferably 70%, more preferably at least 80%, particularly preferably at least 90%, very particularly preferably at least 95%, most preferably 99%, with the sequence of the ε-cyclase promoter shown in SEQ ID NO: 1, 7 or 8, where the homology extends over a length of at least 100 base pairs, preferably at least 200 base pairs, particularly preferably of at least 300 base pairs, very particularly preferably of at least 400 base pairs, most preferably of at least 500 base pairs.

It is possible in this connection for the level of expression of 35 the functional equivalents to differ both downwards and upwards from a comparison value. Preference is given in this connection to the sequences whose level of expression, measured on the basis of the transcribed mRNA or the subsequently translated protein, under conditions which are otherwise unchanged differs quantitatively by not more than 50%, preferably 25%, particularly preferably 10%, from a comparison value obtained with the promoters described by SEQ ID NO: 1, 7 or 8. Particularly preferred sequences are those whose level of expression, measured on the basis of the transcribed mRNA or the subsequently translated protein, under conditions which are otherwise unchanged exceeds quantitatively by more than 50%, preferably 100%, particularly preferably 500%, very particularly preferably 1000%, a comparison value obtained with the promoter described by SEQ ID NO: 1, 7 or 8.

Further examples of functionally equivalent promoter sequences employed in the transgenic expression cassettes or transgenic expression vectors of the invention can easily be found for example in various organisms whose genomic sequence is at least partly known, such as, for example, Arabidopsis thaliana, Brassica napus, Nicotiana tabacum, Solanum tuberosum, Helianthus annuus, Linum sativum oder Oryza sativa, followed by homology comparisons in databases. A possible and preferred starting point for this is the coding regions of the gene whose promoter is described by SEQ ID NO: 1, 7 or 8. Starting from, for example, the cDNA sequences of these genes described by SEQ ID NO: 9, 11, 13 or 15 or the protein sequence derived therefrom and described by SEQ ID NO: 10, 12, 14 or 16 it is possible easily to identify, in a manner familiar to the skilled worker, the corresponding homologous genes—and thus the relevant promoter regions, in other plant species by screening databases or gene libraries (using appropriate gene probes).

In a further preferred embodiment, functional equivalents to the ε-cyclase promoter as shown in SEQ ID NO: 1, 7 or 8 include sequences which are located in a plant organism in the 5′ direction in front of a genomic sequence which codes for an ε-cyclase.

ε-Cyclase means in general all proteins which have an ε-cyclase activity.

By ε-cyclase activity is meant the enzymic activity of an ε-cyclase.

An ε-cyclase means a protein which has the enzymatic activity of converting a terminal linear lycopene residue into an ε-ionone ring.

In particular, ε-cyclase means in general all proteins able to catalyze the cyclization of lycopene to δ-carotene (and where appropriate further to ε-carotene) and/or of neurosporene to α-zeacarotene. The ε-cyclase preferably has an oxidoreductase activity and/or naturally shows a predominant localization in the plastids, especially the chloroplasts and chromoplasts.

An ε-cyclase preferably means a protein having the enzymatic activity for converting lycopene into δ-carotene. Accordingly, ε-cyclase activity means the amount of lycopene converted by the ε-cyclase protein, or the amount of δ-carotene formed, in a particular time.

The ε-cyclase activity in genetically modified plants of the invention and in wild-type or reference plants is preferably determined under the following conditions:

the ε-cyclase activity can be determined by the method of Fraser and Sandmann (Biochem. Biophys. Res. Comm. 185(1) (1992) 9-15) in vitro if potassium phosphate as buffer (pH 7.6), lycopene as substrate, stromal protein of paprika, NADP+, NADPH and ATP are added to a defined amount of plant extract.

The ε-cyclase activity in genetically modified plants of the invention and in wild-type and reference plants is particularly preferably determined by the method of Bouvier, d'Harlingue and Camara (Arch Biochem Biophys 346(1) (1997) 53-64): the in vitro assay is carried out in a volume of 0.25 ml. The mixture contains 50 μM potassium phosphate (pH 7.6), various amounts of plant extract, 20 nM lycopene, 0.25 mg of paprika chromoplastid stromal protein, 0.2 μM NADP+, 0.2 μM NADPH and 1 μM ATP. NADP/NADPH and ATP are dissolved in 0.01 ml of ethanol with 1 mg of Tween 80 immediately before addition to the incubation medium. After a reaction time of 60 minutes at 30° C., the reaction is stopped by adding chloroform/methanol (2:1). The reaction products extracted into chloroform are analyzed by HPLC.

An alternative assay with radioactive substrate is described in Fraser and Sandmann (Biochem Biophys Res Comm 185(1) (1992) 9-15). A further analytical method is described in Beyer, Kröncke and Nievelstein (J Biol Chem 266(26) (1991) 17072-17078).

In a preferred embodiment of the invention, functional equivalents of the ε-cyclase promoter described by SEQ ID NO: 1, 7 or 8 include all promoters which are located in a plant organism in the 5′ direction in front of a genomic sequence which codes for an ε-cyclase having a homology of at least 60%, preferably at least 80%, particularly preferably at least 90%, most preferably at least 95%, with a protein as shown in SEQ ID NO: 10, 12, 14 or 16, where said promoters represent the natural promoter of said genomic sequence.

Functional equivalents of ε-cyclase promoter described by SEQ ID NO: 1, 7 or 8 particularly preferably include all promoters which are located in a plant organism in the 5′ direction in front of a genomic sequence which codes for a nucleic acid sequence whose derived cDNA has a homology of at least 60%, preferably at least 80%, particularly preferably at least 90%, most preferably at least 95%, with the nucleic acid sequence as shown in SEQ ID NO: 9, 11, 13 or 15, where said promoters represent the natural promoter of said genomic sequence, and the cDNA codes for an ε-cyclase.

Preferred promoters include a sequence region of least 250 base pairs, preferably at least 500 base pairs, particularly preferably 1000 base pairs, most preferably at least 2000 base pairs, in the 5′ direction calculated from the ATG start codon of said genomic sequences.

Functional equivalents of the ε-cyclase promoter described by SEQ ID NO: 1, 7 or 8 are particularly preferably all promoters which are located in a plant organism in the 5′ direction in front of a genomic sequence which codes for an ε-cyclase which comprises at least one of the following sequence motifs:

| | |
|---|---|
| 1. G(G/C)GPAGL(A/S) (V/L)A | (SEQ ID NO: 17) |
| 2. (L/I) (N/G/S)RXYG(K/R) (V/L) | (SEQ ID NO: 18) |
| 3. MVFMD(Y/W)RD | (SEQ ID NO: 19) |
| 4. PTFLY(A/V)M(P/A) | (SEQ ID NO: 20) |
| 5. AXMVHP(S/A)TGY(M/S)V(A/V)R | (SEQ ID NO: 21) |
| 6. LWPXER(R/K)RQRXFF | (SEQ ID NO: 22) |

Very particularly preferred functional equivalents of the promoter described by SEQ ID NO: 1, 7 or 8 are those promoters which are located in a plant organism in the 5′ direction in front of a genomic sequence which codes for a protein, where said protein includes at least one of the following sequences:
1. the homologous sequence (H1) from Lactuca sativa as shown in SEQ ID NO: 24,
2. the homologous sequences (H2 and H3) from Adonis palaestina as shown in SEQ ID NO: 26 or 28,
3. the homologous sequence (H4) from Arabidopsis thaliana as shown in SEQ ID NO: 30
4. the homologous sequences (H5 and H6) from Citrus x paradisi as shown in SEQ ID NO: 32 or 34
5. the homologous sequence (H7) from Citrus sinensis as shown in SEQ ID NO: 36
6. the homologous sequence (H8) from Spinacea oleracea as shown in SEQ ID NO: 38
7. the homologous sequence (H9) from Solanum tuberosum as shown in SEQ ID NO: 40
8. the homologous sequences (H10 and H11) from Daucus carota as shown in SEQ ID NO: 42 or 44
9. the homologous sequence (H12) from tomato as shown in SEQ ID NO: 46

Most preferred functional equivalents of the promoter described by SEQ ID NO: 1, 7 or 8 are those promoters which are located in a plant organism in the 5′ direction in front of a genomic sequence which codes for a nucleic acid sequence whose derived cDNA includes at least one of the following sequences:
1. the homologous sequence (H1) from Lactuca sativa as shown in SEQ ID NO: 23,
2. the homologous sequences (H2 and H3) from Adonis palaestina as shown in SEQ ID NO: 25 or 27,
3. the homologous sequence (H4) from Arabidopsis thaliana as shown in SEQ ID NO: 29
4. the homologous sequences (H5 and H6) from Citrus x paradisi as shown in SEQ ID NO: 31 or 33
7. the homologous sequence (H7) from Citrus sinensis as shown in SEQ ID NO: 35
5. the homologous sequence (H8) from Spinacea oleracea as shown in SEQ ID NO: 37
6. the homologous sequence (H9) from Solanum tuberosum as shown in SEQ ID NO: 39
8. the homologous sequences (H10 and H11) from Daucus carota as shown in SEQ ID NO: 41 or 43
9. the homologous sequence (H12) from tomato as shown in SEQ ID NO: 45.

Further examples of functionally equivalent promoter sequences employed in the transgenic expression cassettes or transgenic 45 expression vectors of the invention can easily be found for example in various organisms whose genomic sequence is at least partly known, such as, for example, Arabidopsis thaliana, Brassica napus, Nicotiana tabacum, Solanum tuberosum, Helianthus annuus, Linum sativum, followed by homology comparisons in databases.

A further aspect of the invention relates to the use of at least one nucleic acid sequence or of a part thereof in methods for identifying and/or isolating promoters of genes which code for said nucleic acid sequence, where said nucleic acid sequence codes for an amino acid sequence which includes at least one sequence motif as shown in SEQ ID NO: 17, 18, 19, 20, 21 or 22 or a variation indicated for these sequence motifs. Said nucleic acid sequence preferably codes for an amino acid sequence including a sequence as shown in SEQ ID NO: 24, 26, 28, 30, 32, 34, 36, 38, 40, 42, 44 or 46. Said nucleic acid sequence particularly preferably includes a sequence as shown in SEQ ID NO: 23, 25, 27, 29, 29, 31, 33, 35, 37, 39, 41, 43 or 45. “Part” means in relation to the nucleic acid sequence preferably a sequence of at least 10 bases, preferably 15 bases, particularly preferably 20 bases, most preferably 30 bases.

Further included according to the invention are methods for identifying and/or isolating promoters of genes which code for a promoter having specificity for the flower of plants, where at least one nucleic acid sequence or a part thereof is employed in the identification and/or isolation, where said nucleic acid sequence codes for an amino acid sequence which includes at least one sequence motif as shown in SEQ ID NO: 17, 18, 19, 20, 21 or 22 or a variation indicated for these sequence motifs. Said nucleic acid sequence preferably codes for an amino acid sequence including a sequence as shown in SEQ ID NO: 24, 26, 28, 30, 32, 34, 36, 38, 40, 42, 44 or 46. Said nucleic acid sequence particularly preferably includes a sequence as shown in SEQ ID NO: 23, 25, 27, 29, 29, 31, 33, 35, 37, 39, 41, 43 or 45. “Part” means in relation to the nucleic acid sequence preferably a sequence of at least 10 bases, preferably 15 bases, particularly preferably 20 bases, most preferably 30 bases. In a preferred embodiment, the method of the invention is based on the polymerase chain reaction, where said nucleic acid sequence or a part thereof is employed as primer.

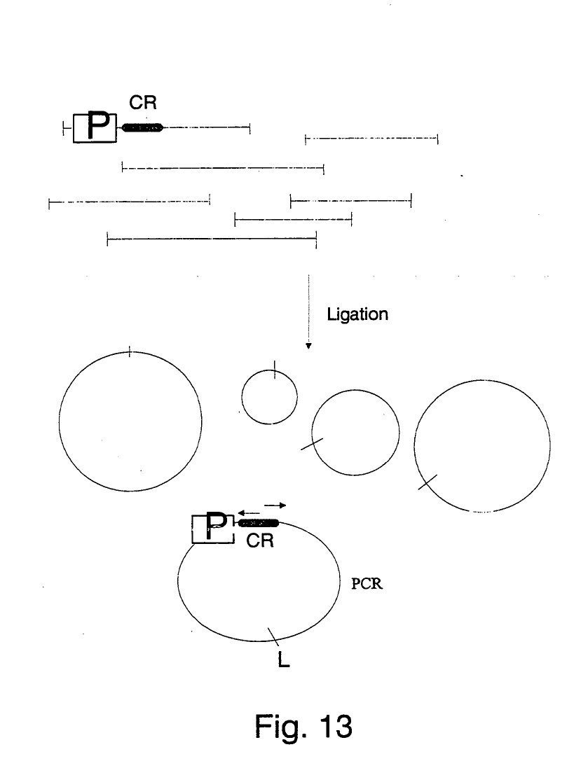

Various methods for identifying and isolating, starting from a nucleic acid sequence (e.g. a gene transcript such as, for example, a cDNA), the promoter of the corresponding gene are known to the skilled worker. In principle, all methods for amplifying flanking chromosomal sequences are available for example for this purpose. The two most commonly used methods are inverse PCR (“iPCR”; diagrammatically depicted in FIG. 13) and “thermal asymmetric interlaced PCR” (“TAIL PCR”). Also suitable in addition is the method of PCR walkings (Devic et al. (1997) Plant Physiol Biochem 35:331-339).

For the iPCR, genomic DNA of the organism from which the functionally equivalent promoter is to be isolated is completely digested with a given restriction enzyme, and then the individual fragments are religated, i.e. linked to themselves to give a circular molecule, in a diluted mixture. The large number of resulting circular DNA molecules also includes those comprising the known sequence (for example the sequence coding for the homologous protein). Starting from this, the circular molecule can be amplified by PCR using a primer pair where both primers are able to anneal to the known sequence segment. One possible embodiment of the iPCR is reproduced in example 2.

The TAIL-PCR is based on the use of firstly a set of successively truncated highly specific primers which anneal to the known genomic sequence (for example the sequence coding for the homologous protein), and secondly a set of shorter random primers with a lower melting temperature, so that a less sequence-specific annealing to genomic DNA flanking the known genomic sequence takes place. Annealing of the primers to the DNA to be amplified is possible with such a primer combination to make specific amplification of the desired target sequence possible. One possible embodiment of the TAIL-PCR is reproduced for example in example 2.

A further aspect of the invention relates to methods for preparing a transgenic expression cassette having specificity for the flowers of plants, including the following steps:
I. isolation of a promoter sequence, where at least one nucleic acid sequence or a part thereof is employed in the isolation, where said nucleic acid sequence codes for an amino acid sequence which includes at least one sequence motif as shown in SEQ ID NO: 17, 18, 19, 20, 21 or 22 or a variation indicated for these sequence motifs.
II. functional linkage of said promoter sequence to a further nucleic acid sequence, where said nucleic acid sequence is heterologous in relation to the promoter.

Said nucleic acid sequence preferably codes for an amino acid sequence including a sequence as shown in SEQ ID NO: 24, 26, 28, 30, 32, 34, 36, 38, 40, 42, 44 or 46. Said nucleic acid sequence particularly preferably includes a sequence as shown in SEQ ID NO: 23, 25, 27, 29, 29, 31, 33, 35, 37, 39, 41, 43 or 45. “Part” means in relation to the nucleic acid sequence preferably a sequence of at least 10 bases, preferably 15 bases, particularly preferably 20 bases, most preferably 30 bases. In a preferred embodiment, the method of the invention is based on the polymerase chain reaction, where said nucleic acid sequence or a part thereof is employed as primer. Methods known to the skilled worker, such as, for example, ligation etc., can be employed for the functional linkage (see below).

The level of expression of a functionally equivalent promoter can be both downwards and upwards compared with the promoter found in SEQ ID NO: 1, 7 or 8. Preference is given in this connection to the sequences whose level of expression, measured on the basis of the transcribed mRNA or the subsequently translated protein, under conditions which are otherwise unchanged differs quantitatively by not more than 50%, preferably 25%, particularly preferably 10%, from a comparison value obtained with the promoters described by SEQ ID NO: 1, 7 or 8. Particularly preferred sequences are those whose level of expression, measured on the basis of the transcribed mRNA or the subsequently translated protein, under conditions which are otherwise unchanged exceeds quantitatively by more than 50%, preferably 100%, particularly preferably 500%, very particularly preferably 1000%, a comparison value obtained with the promoter described by SEQ ID NO: 1, 7 or 8. The preferred comparison value is the level of expression of the mRNAs, naturally expressed from the promoter, of an ε-cyclase or of the protein resulting therefrom. Also preferred as comparison value is the level of expression obtained with any defined nucleic acid sequence, preferably nucleic acid sequences which code for easily quantifiable proteins. Very particular preference is given in this connection to reporter proteins (Schenborn E & Groskreutz D (1999) Mol Biotechnol 13(l):29-44) such as the green fluorescent protein (GFP) (Chiu W L et al. (1996) Curr Biol 6:325-330; Leffel S M et al. (1997) Biotechniques. 23(5):912-8), chloramphenicol acetyltransferase, a luciferase (Millar et al. (1992) Plant Mol Biol Rep 10:324-414) or β-glucuronidase, very particularly preferably β-glucuronidase (Jefferson et al. (1987) EMBO J 6:3901-3907).

Functional equivalents also include natural or artificial mutations of the promoter sequence described in SEQ ID NO: 1, 7 or 8. Mutations include substitutions, additions, deletions, inversions or insertions of one or more nucleotide residues. Thus, for example, the present invention also includes nucleotide sequences obtained by modification of the ε-cyclase promoter as shown in SEQ ID NO: 1, 7 or 8. The aim of such a modification may be further localization of the sequence contained therein or, for example, also the insertion or deletion of restriction endonuclease cleavage sites, the deletion of excess DNA or the addition of further sequences, for example further regulatory sequences.

Where insertions, deletions or substitutions, such as, for example, transitions and transversions, are appropriate, it is possible to use techniques known per se, such as in vitro mutagenesis, primer repair, restriction or ligation. Transition means a base-pair exchange of a purine/pyrimidine pair into another purine/pyrimidine pair (e.g. A-T for G-C). Transversion means a base-pair exchange of a purine/pyrimidine pair for a pyrimidine/purine pair (e.g. A-T for T-A). Deletion means removal of one or more base pairs. Insertion means introduction of one or more base pairs.

Complementary ends of the fragments for ligation can be made available by manipulations such as, for example, restriction, chewing back or filling in of overhangs for blunt ends. Analogous results are also obtainable by using the polymerase chain reaction (PCR) using specific oligonucleotide primers.

Homology between two nucleic acids means the identity of the nucleic acid sequence over the complete sequence length in each case, which is calculated by comparison with the aid of the GAP program algorithm (Wisconsin Package Version 10.0, University of Wisconsin, Genetics Computer Group (GCG), Madison, USA), setting the following parameters:

| | |
|---|---|
| Gap Weight: 12 | Length Weight: 4 |
| Average Match: 2.912 | Average Mismatch: −2.003 |

For example, a sequence which has a homology of at least 50% based on nuleic acids with the sequence SEQ ID NO: 1 means a sequence which has a homology of at least 50% on comparison with the sequence SEQ ID NO: 1 by the above program algorithm with the above set of parameters.

Homology between two polypeptides means the identity of the amino acid sequence over the respective sequence length, which is calculated by comparison with the aid of the GAP program algorithm (Wisconsin Package Version 10.0, University of Wisconsin, Genetics Computer Group (GCG), Madison, USA), setting the following parameters:

| | |
|---|---|
| Gap Weight: 8 | Length Weight: 2 |
| Average Match: 2.912 | Average Mismatch: −2.003 |

For example, a sequence having a homology of at least 60% based on protein with the sequence SEQ ID NO: 10 means a sequence which has a homology of at least 60% on comparison with the sequence SEQ ID NO: 10 by the above program algorithm with the above set of parameters.

Functional equivalents also means DNA sequences which hybridize under standard conditions with the nucleic acid sequence coding for the ε-cyclase promoter as shown in SEQ ID NO: 1, 7 or 8, or with the nucleic acid sequences complementary thereto, and which have substantially the same promoter properties. The term standard hybridization conditions is to be understood broadly and means both stringent and less stringent hybridization conditions. Such hybridization conditions are described inter alia in Sambrook J, Fritsch E F, Maniatis T et al., in Molecular Cloning—A Laboratory Manual, 2^{nd} edition, Cold Spring Harbor Laboratory Press, 1989, pages 9.31-9.57 or in Current Protocols in Molecular Biology, John Wiley & Sons, N.Y. (1989), 6.3.1-6.3.6.

For example, the conditions during the washing step can be selected from the range of conditions limited by those of low stringency (with approximately 2×SSC at 50° C.) and of high stringency (with approximately 0.2×SSC at 50° C., preferably at 65° C.) (20×SSC: 0.3 M sodium citrate, 3 M NaCl, pH 7.0). In addition, the temperature during the washing step can be raised from low-stringency conditions at room temperature, approximately 22° C., to more stringent conditions at approximately 65° C. Both parameters, the salt concentration and the temperature, can be varied simultaneously, and it is also possible for one of the two parameters to be kept constant and only the other to be varied. It is also possible to employ denaturing agents such as, for example, formamide or SDS during the hybridization. Hybridization in the presence of 50% formamide is preferably carried out at 42° C. Some exemplary conditions for hybridization and washing steps are given below:
(1) Hybridization conditions with for example
   a) 4×SSC at 65° C., or
   b) 6×SSC, 0.5% SDS, 100 μg/ml denatured fragmented salmon sperm DNA at 65° C., or
   c) 4×SSC, 50% formamide, at 42° C., or
   d) 2× or 4×SSC at 50° C. (low-stringency condition), or
   e) 2× or 4×SSC, 30 to 40% formamide at 42° C. (low-stringency condition), or
   f) 6×SSC at 45° C., or,
   g) 0.05 M sodium phosphate buffer pH 7.0, 2 μM EDTA, 1% BSA and 7% SDS.
(2) washing steps with for example
   a) 0.1×SSC at 65° C., or
   b) 0.1×SSC, 0.5% SDS at 68° C., or
   c) 0.1×SSC, 0.5% SDS, 50% formamide at 42° C., or
   d) 0.2×SSC, 0.1% SDS at 42° C., or
   e) 2×SSC at 65° C. (low-stringency condition), or
   f) 40 μM sodium phosphate buffer pH 7.0, 1% SDS, 2 μM EDTA.

Methods for preparing functional equivalents of the invention preferably include the introduction of mutations into the ε-cyclase promoter as shown in SEQ ID NO: 1, 7 or 8. Mutagenesis may be random, in which case the mutagenized sequences are subsequently screened for their properties by a trial and error procedure. Particularly advantageous selection criteria include for example the level of the resulting expression of the introduced nucleic acid sequence in a flower tissue.

Methods for mutagenesis of nucleic acid sequences are known to the skilled worker and include by way of example the use of oligonucleotides with one or more mutations compared with the region to be mutated (e.g. in a site-specific mutagenesis).

Primers with approximately 15 to approximately 75 nucleotides or more are typically employed, with preferably about 10 to about 25 or more nucleotide residues being located on both sides of the sequence to be modified. Details and procedure for said mutagenesis methods are familiar to the skilled worker (Kunkel et al. (1987) Methods Enzymol 154:367-382; Tomic et al. (1990) Nucl Acids Res 12:1656; Upender et al. (1995) Biotechniques 18(1):29-30; U.S. Pat. No. 4,237,224). A mutagenesis can also be achieved by treating for example transgenic expression vectors comprising one of the nucleic acid sequences of the invention with mutagenizing agents such as hydroxylamine.

An alternative possibility is to delete nonessential sequences of a promoter of the invention without significantly impairing the essential properties mentioned. Such deletion variants represent functionally equivalent fragments to the promoters described by SEQ ID NO: 1, 7 or 8 or to functional equivalents thereof. Localization of the promoter sequence to particular essential regulatory regions can be carried out for example with the aid of search routine to search for promoter elements. Particular promoter elements are often present in increased numbers in the regions relevant for promoter activity. This analysis can be carried out for example with computer programs such as the PLACE program (“Plant Cis-acting Regulatory DNA Elements”; Higo K et al. (1999) Nucl Acids Res 27(1): 297-300), the BIOBASE database “Transfac” (Biologische Datenbanken GmbH, Braunschweig; Wingender E et al. (2001) Nucleic Acids Res 29(l):281-3) or the PlantCARE database (Lescot M et al. (2002) Nucleic Acids Res 30(1):325-7).

The functionally equivalent fragments of one of the promoters of the invention—for example of the ε-cyclase promoters described by SEQ ID NO: 1, 7 or 8—preferably include at least 200 base pairs, very particularly preferably at least 500 base pairs, most preferably at least 1000 base pairs of the 3′ end of the respective promoter of the invention—for example the promoters described by SEQ ID NO: 1, 7 or 8—the length being calculated from the translation start (“ATG” codon) upstream in the 5′ direction.

Further functionally equivalent fragments may be generated for example by deleting any 5′-untranslated regions still present. For this purpose, the start of transcription of the corresponding genes can be determined by methods familiar to the skilled worker (such as, for example, 5′-RACE), and the 5′-untranslated regions can be deleted by PCR-mediated methods or endonuclease digestion. Thus, for example, the 5′-untranslated regions included in the promoters shown in SEQ ID NO: 7 or 8 can be deleted without the promoter losing its essential functionality. Corresponding deletion variants are expressly included as functional equivalents.

In transgenic expression cassettes of the invention, at least one of the promoters of the invention (e.g. described by SEQ ID NO: 1, 7 or 8) is functionally linked to at least one nucleic acid sequence to be expressed transgenically.

A functional linkage means, for example, the sequential arrangement of one of the promoters of the invention (e.g. described by SEQ ID NO: 1, 7 or 8) with a nucleic acid sequence to be expressed transgenically and, where appropriate, further genetic control sequences such as, for example, a terminator or a polyadenylation sequence in such a way that the promoter is able to fulfill its function in the transgenic expression of the nucleic acid sequence under suitable conditions, and expression of the nucleic acid sequence (i.e. transcription and, where appropriate, translation) takes place. “Suitable conditions” means in this connection preferably the presence of the expression cassette in a plant cell, preferably a plant cell included in a flower of the plant.

Arrangements in which the nucleic acid sequence to be expressed transgenically is positioned behind one of the promoters of the invention (e.g. described by SEQ ID NO: 1, 7 or 8), so that the two sequences are covalently connected together, are preferred. In this connection, the distance between the promoter sequence and the nucleic acid sequence to be expressed transgenically is preferably fewer than 200 base pairs, particularly preferably less than 100 base pairs, very particularly preferably less than 50 base pairs.

Production of a functional linkage and production of a transgenic expression construct can be achieved by means of conventional recombination and cloning techniques as described for example in Maniatis T, Fritsch E F and Sambrook J (1989) Molecular Cloning: A Laboratory Manual, Cold Spring Harbor Laboratory, Cold Spring Harbor (N.Y.), in Silhavy T J, Berman M L and Enquist L W (1984) Experiments with Gene Fusions, Cold Spring Harbor Laboratory, Cold Spring Harbor (N.Y.) and in Ausubel F M et al. (1987) Current Protocols in Molecular Biology, Greene Publishing Assoc. and Wiley Interscience. However, further sequences which have for example the function of a linker with particular restriction enzyme cleavage sites or of a signal peptide may also be positioned between the two sequences. Insertion of sequences may also lead to expression of fusion proteins. It is possible and preferred for the transgenic expression construct, consisting of a linkage of promoter and nucleic acid sequence to be expressed, to be integrated into a vector and be inserted into a plant genome for example by transformation.

However, an expression cassette also means constructions in which one of the promoters of the invention (e.g. described by SEQ ID NO: 1, 7 or 8) is, without necessarily having been functionally linked beforehand to a nucleic acid sequence to be expressed, introduced into a host genome, for example by targeted homologous recombination or random insertion, there undertakes regulatory control over endogenous nucleic acid sequences then functionally linked thereto, and controls the transgenic expression thereof. Insertion of the promoter—for example by a homologous recombination—in front of a nucleic acid coding for a particular polypeptide results in an expression cassette of the invention which controls the targeted expression of the particular polypeptide in the flower of plants. It is also possible for example for the natural promoter of an endogenous gene to be replaced by one of the promoters of the invention (e.g. described by SEQ ID NO: 1, 7 or 8), and for the expression behavior of the endogenous gene to be modified.

A further possibility is also for the promoter to be inserted in such a way that antisense RNA or a double-stranded RNA (e.g. in the form of an inverted repeat) is expressed to give the nucleic acid coding for a particular polypeptide. In this way, expression of the particular polypeptide in the flower of plants is selectively downregulated or switched off.

It is also possible analogously for a nucleic acid sequence which is to be expressed transgenically to be placed—for example by homologous recombination—behind the sequence which codes for one of the promoters of the invention (e.g. described by SEQ ID NO: 1, 7 or 8), and which is located in its natural chromosomal context, so as to result in an expression cassette of the invention which controls the expression of the nucleic acid sequence to be expressed transgenically in the flower of plants.

The transgenic expression cassettes of the invention may include further genetic control sequences. The term genetic control sequences is to be understood broadly and means all sequences having an influence on the coming into existence or the function of a transgenic expression cassette of the invention. Genetic control sequences modify for example the transcription and translation in prokaryotic or eukaryotic organisms. The transgenic expression cassettes of the invention preferably include as additional genetic control sequence a terminator sequence 3′-downstream from the particular nucleic acid sequence to be expressed transgenically, and where appropriate further customary regulatory elements, in each case functionally linked to the nucleic acid sequence to be expressed transgenically.

Genetic control sequences also include further promoters, promoter elements or minimal promoters able to modify the expression-controlling properties. It is thus possible for example through genetic control sequences for tissue-specific expression to take place additionally in dependence on particular stress factors. Corresponding elements are described for example for water stress, abscisic acid (Lam E and Chua N H, J Biol Chem 1991; 266(26):17131-17135) and heat stress (Schoffl F et al. (1989) Mol Gen Genetics 217(2-3):246-53).

A further possibility is for further promoters which make transgenic expression possible in further plant tissues or in other organisms such as, for example, E. coli bacteria to be functionally linked to the nucleic acid sequence to be expressed. Suitable promoters are in principle all promoters functional in plants. Promoters functional in plants means in principle every promoter able to control the expression of genes, in particular foreign genes, in plants or plant parts, cells, tissues, cultures. It is moreover possible for expression to be for example constitutive, inducible or development-dependent.

Preference is given to constitutive promoters, tissue-specific promoters, development-dependent promoters, chemically inducible, stress-inducible or pathogen-inducible promoters. Corresponding promoters are generally known to the skilled worker.

Further advantageous control sequences are to be found for example in the promoters of gram-positive bacteria such as amy and SPO2 or in the yeast or fungal promoters ADCl, MFα, AC, P-60, CYC1, GAPDH, TEF, rp28, ADH.

It is possible in principle for all natural promoters with their regulatory sequences like those mentioned above to be used for the method of the invention. It is additionally also possible for synthetic promoters to be used advantageously.

Genetic control sequences further include also the 5′-untranslated regions, introns or noncoding 3′ region of genes such as, for example, the actin-1 intron, or the Adhl-S introns 1, 2 and 6 (generally: The Maize Handbook, Chapter 116, Freeling and Walbot, Eds., Springer, New York (1994)), preferably the genes with the gene locus At2g46720, At3g01980 and At1g63140 from Arabidopsis thaliana. It is possible to show that such regions may have a significant function in regulating gene expression. Thus, it has been shown that 5′-untranslated sequences are able to enhance the transient expression of heterologous genes. Examples of translation enhancers which may be mentioned are the 5′ leader sequence from tobacco mosaic virus (Gallie et al. (1987) Nucl Acids Res 15:8693-8711) and the like. They may in addition promote tissue specificity (Rouster J et al. (1998) Plant J 15:435-440). The nucleic acid sequences indicated in SEQ ID NO: 2, 7 or 8 in each case represent the promoter region and the 5′-untranslated regions up to the ATG start codon of the respective genes.

The transgenic expression construct may advantageously comprise one or more so-called enhancer sequences functionally linked to the promoter, which make increased transgenic expression of the nucleic acid sequence possible. Additional advantageous sequences can also be inserted at the 3′ end of the nucleic acid sequences to be expressed transgenically, such as further regulatory elements or terminators. The nucleic acid sequences to be expressed transgenically may be present in one or more copies in the gene construct.

Polyadenylation signals suitable as control sequences are plant polyadenylation signals, preferably those which are essentially T-DNA polyadenylation signals from Agrobacterium tumefaciens. Examples of particularly suitable terminator sequences are the OCS (octopine synthase) terminator and the NOS (nopaline synthase) terminator.

Control sequences additionally mean those which make homologous recombination or insertion into the genome of a host organism possible or allow deletion from the genome. In homologous recombination for example the coding sequence of a particular endogenous gene can be specifically replaced by a sequence coding for a dsRNA. Methods such as cre/lox technology permit tissue-specific, and in some circumstances inducible, deletion of the transgenic expression constant from the genome of the host organism (Sauer B (1998) Methods 14(4):381-92). In this case, particular flanking sequences are attached to the target gene (lox sequences) and make later deletion by means of cre recombinase possible.

A transgenic expression cassette and/or the transgenic expression vectors derived therefrom may comprise further functional elements. The term functional element is to be understood broadly and means all elements which have an influence on the production, replication or function of the transgenic expression constructs of the invention, of the transgenic expression vectors or of the transgenic organisms. Non-restrictive examples which may be mentioned are:
a) Selection markers which confer resistance to biocides such as metabolism inhibitors (e.g. 2-deoxyglucose 6-phosphate;

WO 98/45456), antibiotics (e.g. kanamycin, G 418, bleomycin, hygromycin) or—preferably—herbicides (e.g. phosphinothricin). Examples of selection markers which may be mentioned are: phosphinothricin acetyltransferases (bar and pat gene), which inactivate glutamine synthase inhibitors, 5-enolpyruvylshikimate-3-phosphate synthases (EPSP synthase genes) which confer resistance to glyphosate (N-(phosphonomethyl)glycine), glyphosate-degrading enzymes (gox gene product; glyphosate oxidoreductase), dehalogenases which for example inactivate dalapon (deh gene product), sulfonylurea- and imidazolinone-inactivating acetolactate synthases, and nitrilases which for example degrade bromoxynil (bxn gene product), the aasa gene product which confers resistance to the antibiotic spectinomycin, streptomycin phosphotransferases (SPT) which ensure resistance to streptomycin, neomycin phosphotransferases (NPTII) which confer resistance to kanamycin or geneticin, the hygromycin phosphotransferases (HPT) which mediate resistance to hygromycin, the acetolactate synthases (ALS) which confer resistance to sulfonylurea herbicides (e.g. mutated ALS variants with, for example, the S4 and/or Hra mutation).
b) Reporter genes which code for easily quantifiable proteins and ensure via an intrinsic color or enzymic activity an assessment of the transformation efficiency or of the location or timing of expression. Very particular preference is given in this connection to reporter proteins (Schenborn E, Groskreutz D. Mol Biotechnol. 1999; 13(l):29-44) such as the green fluorescent protein (GFP) (Sheen et al.(1995) Plant Journal 8(5):777-784), the chloramphenicol acetyltransferase, a luciferase (Ow et al. (1986) Science 234:856-859), the aequorin gene (Prasher et al. (1985) Biochem Biophys Res Commun 126(3):1259-1268), the β-galactosidase, with very particular preference for β-glucuronidase (Jefferson et al. (1987) EMBO J 6:3901-3907).
c) Origins of replication which ensure replication of the transgenic expression constructs or transgenic expression vectors of the invention in, for example, E. coli. Examples which may be mentioned are ORI (origin of DNA replication), the pBR322 ori or the P15A ori (Sambrook et al.: Molecular Cloning. A Laboratory Manual, 2^{nd} ed. Cold Spring Harbor Laboratory Press, Cold Spring Harbor, N.Y., 1989).
d) Elements which are necessary for agrobacterium-mediated plant transformation, such as, for example, the right or left border of the T-DNA or the vir region.

“Introduction” includes for the purposes of the invention all methods suitable for introducing a nucleic acid sequence (for example an expression cassette of the invention) directly or indirectly into an organism (e.g. a plant) or a cell, compartment, tissue, organ or propagation material (e.g. seeds or fruits) thereof, or for generating such therein. Direct and indirect methods are included. The introduction can lead to a temporary (transient) presence of said nucleic acid sequence or else to a permanent (stable) presence. Introduction includes for example methods such as transfection, transduction or transformation. The organisms used in the methods are grown or cultured, depending on the host organism, in the manner known to the skilled worker.

Introduction of a transgenic expression cassette of the invention into an organism or cells, tissues, organs, parts or seeds thereof (preferably into plants or plant cells, tissues, organs, parts or seeds) can advantageously be achieved by use of vectors comprising the transgenic expression cassettes. Vectors may be for example plasmids, cosmids, phages, viruses or else agrobacteria. The transgenic expression cassettes can be inserted into the vector (preferably a plasmid vector) via a suitable restriction cleavage site. The resulting vector can be firstly introduced and amplified in E. coli. Correctly transformed E. coli are selected and cultured, and the recombinant vector is isolated by methods familiar to the skilled worker. Restriction analysis and sequencing can be used to check the cloning step. Preferred vectors are those making stable integration of the expression cassette into the host genome possible.

Production of a transformed organism (or of a transformed cell or tissue) requires introduction of the appropriate DNA (e.g. the expression vector) or RNA into the appropriate host cell. A large number of methods is available for this process, which is referred to as transformation (or transduction or transfection) (Keown et al. (1990) Methods in Enzymology 185:527-537). Thus, the DNA or RNA can for example be introduced directly by microinjection or by bombardment with DNA-coated microparticles. The cell can also be permeabilized chemically, for example with polyethylene glycol, so that the DNA is able to enter the cell by diffusion. The DNA introduction can also take place by protoplast fusion with other DNA-containing units such as minicells, cells, lysosomes or liposomes. Electroporation is another suitable method for introducing DNA, in which the cells are reversibly permeabilized by an electrical impulse. Corresponding methods are described (for example in Bilang et al. (1991) Gene 100:247-250; Scheid et al. (1991) Mol Gen Genet 228:104-112; Guerche et al. (1987) Plant Science 52:111-116; Neuhause et al. (1987) Theor Appl Genet 75:30-36; Klein et al. (1987) Nature 327:70-73; Howell et al. (1980) Science 208:1265; Horsch et al.(1985) Science 227:1229-1231; DeBlock et al. (1989) Plant Physiology 91:694-701; Methods for Plant Molecular Biology (Weissbach and Weissbach, eds.) Academic Press Inc. (1988); and Methods in Plant Molecular Biology (Schuler and Zielinski, eds.) Academic Press Inc. (1989)).

Vectors preferred for expression in E. coli are pQE70, pQE60 and pQE-9 (QIAGEN, Inc.); pBluescript vectors, Phagescript vectors, pNH8A, pNH16a, pNH18A, pNH46A (Stratagene Cloning Systems, Inc.); ptrc99a, pKK223-3, pKK233-3, pDR540, pRIT5 (Pharmacia Biotech, Inc.).

Preferred vectors for expression in mammalian cells include PWLNE0, pSV2CAT, pOG44, pXT1 and pSG (Stratagene Inc.); pSVK3, pBPV, PMSG and pSVL (Pharmacia Biotech, Inc.). Inducible vectors which may be mentioned are pTet-tTak, pTet-Splice, pcDNA4/TO, pcDNA4/TO /LacZ, pcDNA6/TR, pcDNA4/TO/Myc-His/LacZ, pcDNA4/TO/Myc-His A, pcDNA4/TO/Myc-His B, pcDNA4/TO/Myc-His C, pVgRXR (Invitrogen, Inc.) or the pMAM series (Clontech, Inc.; GenBank Accession No: U02443). These themselves provide the inducible regulatory control element for example for a chemically inducible expression.

Vectors for expression in yeast include for example pYES2, pYD1, pTEF1/Zeo, pYES2/GS, PPICZ, pGAPZ, pGAPZalph, pPIC9, pPIC3.5, PHIL-D2, PHIL-S1, pPIC3SK, pPIC9K, and PA0815 (Invitrogen, Inc.).

Cloning vectors and techniques for genetic manipulation of ciliates and algae are known to the skilled worker (WO 98/01572; Falciatore et al. (1999) Marine Biotechnology 1(3):239-251; Dunahay et al. (1995) J Phycol 31:10004-1012).

The methods to be used in principle for the transformation of animal cells or of yeast cells are similar to those for “direct” transformation of plant cells. Methods such as calcium phosphate or liposome-mediated transformation or else electroporation are preferred in particular.

Various methods and vectors for inserting genes into the genome of plants and for regenerating plants from plant tissues or plant cells are known (Plant Molecular Biology and Biotechnology (CRC Press, Boca Raton, Fla.), Chapter 6/7, pp. 71-119 (1993); White F F (1993) Vectors for Gene Transfer in Higher Plants; in: Transgenic Plants, Vol. 1, Engineering and Utilization, editors: Kung and Wu R, Academic Press, 15-38; Jenes B et al. (1993) Techniques for Gene Transfer, in: Transgenic Plants, Vol. 1, Engineering and Utilization, editors: Kung and R. Wu, Academic Press, pp. 128-143; Potrykus (1991) Annu Rev Plant Physiol Plant Molec Biol 42:205-225; Halford N G, Shewry P R (2000) Br Med Bull 56(1):62-73). Those mentioned above are included, for example. In the case of plants, the described methods for the transformation and regeneration of plants from plant tissues or plant cells are used for transient or stable transformation. Suitable methods are, in particular, protoplast transformation by polyethylene glycol-induced DNA uptake, calcium phosphate-mediated transformation, DEAE-dextran-mediated transformation, liposome-mediated transformation (Freeman et al. (1984) Plant Cell Physiol. 29:1353ff; U.S. Pat. No. 4,536,475), biolistic methods with the gene gun (“particle bombardment” method; U.S. Pat. No. 5,100,792; EP-A 0 444 882; EP-A 0 434 616; Fromm M E et al. (1990) Bio/Technology 8(9):833-9; Gordon-Kamm et al. (1990) Plant Cell 2:603), electroporation, incubation of dry embryos in DNA-containing solution, electroporation (EP-A 290 395, WO 87/06614), microinjection (WO 92/09696, WO 94/00583, EP-A 0 331 083, EP-A 0 175 966) or other methods of direct DNA introduction (DE 4 005 152, WO 90/12096, U.S. Pat. No. 4,684,611). Physical methods of DNA introduction into plant cells are surveyed in Oard (1991) Biotech Adv 9:1-11.

In the case of these “direct” transformation methods, no particular requirements need be met by the plasmid used. Simple plasmids such as those of the pUC series, pBR322, M13mp series, pACYC184 etc. can be used. If complete plants are to be regenerated from the transformed cells, it is necessary for an additional selectable marker gene to be present on the plasmid.

Besides these “direct” transformation techniques, it is also possible to carry out a transformation by bacterial infection using agrobacterium (e.g. EP 0 116 718), viral infection using viral vectors (EP 0 067 553; U.S. Pat. No. 4,407,956; WO 95/34668; WO 93/03161) or using pollen (EP 0 270 356; WO 85/01856; U.S. Pat. No. 4,684,611).

The transformation is preferably effected using agrobacteria which comprise disarmed Ti plasmid vectors, utilizing their natural ability to transfer genes to plants (EP-A 0 270 355; EP-A 0 116 718).

Agrobacterium transformation is widely used for the transformation of dicotyledons, but is also increasingly being applied to monocotyledons (Toriyama et al. (1988) Bio/Technology 6: 1072-1074; Zhang et al. (1988) Plant Cell Rep 7:379-384; Zhang et al. (1988) Theor Appl Genet 76:835-840; Shimamoto et al. (1989) Nature 338:274-276; Datta et al. (1990) Bio/Technology 8: 736-740; Christou et al. (1991) Bio/Technology 9:957-962; Peng et al. (1991) International Rice Research Institute, Manila, Philippines 563-574; Cao et al. (1992) Plant Cell Rep 11:585-591; Li et al. (1993) Plant Cell Rep 12:250-255; Rathore et al. (1993) Plant Mol Biol 21:871-884; Fromm et al. (1990) Bio/Technology 8:833-839; Gordon-Kamm et al. (1990) Plant Cell 2:603-618; D'Halluin et al. (1992) Plant Cell 4:1495-1505; Walters et al. (1992) Plant Mol Biol 18:189-200; Koziel et al. (1993) Biotechnology 11:194-200; Vasil I K (1994) Plant Mol Biol 25:925-937; Weeks et al. (1993) Plant Physiol 102:1077-1084; Somers et al. (1992) Bio/Technology 10:1589-1594; WO 92/14828; Hiei et al. (1994) Plant J 6:271-282).

The strains mostly used for agrobacterium transformation, Agrobacterium tumefaciens or Agrobacterium rhizogenes comprise a plasmid (Ti or Ri plasmid) which is transferred to the plant after agrobacterium infection. Part of this plasmid, called T-DNA (transferred DNA), is integrated into the genome of the plant cell. Alternatively, binary vectors (mini-Ti plasmids) can also be transferred into plants and integrated in the genome thereof by agrobacterium.

The use of Agrobacterium tumefaciens for the transformation of plants using tissue culture explants is described (inter alia Horsch R B et al. (1985) Science 225:1229ff.; Fraley et al. (1983) Proc Natl Acad Sci USA 80: 4803-4807; Bevans et al. (1983) Nature 304:184-187). Many Agrobacterium tumefaciens strains are able to transfer genetic material—for example the expression cassettes of the invention—such as, for example, the strains EHA101[pEHA101], EHA105[pEHA105], LBA4404[pAL4404], C58Cl[pMP90] and C58Cl[pGV2260] (Hood et al. (1993) Transgenic Res 2:208-218; Hoekema et al. (1983) Nature 303:179-181; Koncz and Schell (1986) Gen Genet 204:383-396; Deblaere et al. (1985) Nucl Acids Res 13: 4777-4788).

On use of agrobacteria, the expression cassette must be integrated into specific plasmids either into a shuttle or intermediate vector or into a binary vector. Binary vectors able to replicate both in E. coli and in agrobacterium are preferably used. They normally comprise a selection marker gene and a linker or polylinker, flanked by the right and left T-DNA border sequence. They can be transformed directly into agrobacterium (Holsters et al. (1978) Mol Gen Genet 163:181-187). The agrobacterium acting as host organsim in this case should already comprise a plasmid having the vir region. This is necessary for transfer of the T-DNA into the plant cell. An agrobacterium transformed in this way can be used to transform plant cells. The use of T-DNA for transforming plant cells has been intensively investigated and described (EP-A 0 120 516; Hoekema, In: The Binary Plant Vector System, Offsetdrukkerij Kanters B. V., Alblasserdam, Chapter V; An et al. (1985) EMBO J 4:277-287). Various binary vectors are known, and some of them are commercially available, such as, for example, pBI101.2 or pBIN19 (Clontech Laboratories, Inc. USA; Bevan et al. (1984) Nucl Acids Res 12:8711), pBinAR, pPZP200 or pPTV.

Agrobacteria transformed with such a vector can then be used in a known manner for transforming plants, especially crop plants such as, for example, oilseed rape, by for example bathing wounded leaves or pieces of leaf in a solution of agrobacteria and then cultivating in suitable media. Transformation of plants by agrobacteria is described (White F F (1993) Vectors for Gene Transfer in Higher Plants; in Transgenic Plants, Vol. 1, Engineering and Utilization, edited by S D Kung and R Wu, Academic Press, pp. 15-38; Jenes B et al.(1993) Techniques for Gene Transfer, in: Transgenic Plants, Vol. 1, Engineering and Utilization, edited by S.D. Kung and R. Wu, Academic Press, pp. 128-143; Potrykus (1991) Annu Rev Plant Physiol Plant Molec Biol 42:205-225). Transgenic plants which have integrated the expression systems of the invention described above can be regenerated in a known manner from the transformed cells of the wounded leaves or pieces of leaf.

Stably transformed cells (i.e. those which have integrated the introduced DNA into the DNA of the host cell) can be selected from untransformed ones if a selectable marker is a constituent of the introduced DNA. Any gene able to confer a resistance to a biocide (e.g. an antibiotic or herbicide, see above) can act as marker, for example. Transformed cells which express such a marker gene are able to survive in the presence of concentrations of a corresponding biocide which kill an untransformed wild type. The selection marker permits the selection of transformed cells from untransformed ones (McCormick et al. (1986) Plant Cell Reports 5:81-84). The resulting plants can be grown and crossed in the usual way. Two or more generations should be cultivated in order to ensure that the genomic integration is stable and heritable.

As soon as a transformed plant cell has been produced, it is possible to obtain a complete plant by using methods known to the skilled worker. These entail, for example, starting from callus cultures, single cells (e.g. protoplasts) or leaf disks (Vasil et al. (1984) Cell Culture and Somatic Cell Genetics of Plants, Vol I, II and III, Laboratory Procedures and Their Applications, Academic Press; Weissbach and Weissbach (1989) Methods for Plant Molecular Biology, Academic Press). The formation of shoot and root from these still undifferentiated callus cell masses can be induced in a known manner. The resulting shoots can be planted out and grown. Corresponding methods are described (Fennell et al. (1992) Plant Cell Rep. 11: 567-570; Stoeger et al (1995) Plant Cell Rep. 14:273-278; Jahne et al. (1994) Theor Appl Genet 89:525-533).

The effectiveness of expression of the transgenically expressed nucleic acids can be estimated for example in vitro by shoot-meristem propagation using one of the selection methods described above. In addition, a change in the type and level of expression of a target gene, and the effect on the phenotype of the plant can be tested on test plants in glasshouse tests.

A further aspect of the invention relates to transgenic organisms transformed with at least one expression cassette of the invention or one vector of the invention, and cells, cell cultures, tissues, parts—such as, for example, in the case of plant organisms leaves, roots etc.—or propagation material derived from such organisms.

By organism, starting or host organisms are meant prokaryotic or eukaryotic organisms such as, for example, microorganisms or plant organisms. Preferred microorganisms are bacteria, yeasts, algae or fungi.

Preferred bacteria are bacteria of the genus Escherichia, Erwinia, Agrobacterium, Flavobacterium, Alcaligenes, Pseudomonas, Bacillus or cyanobacteria, for example of the genus Synechocystis and further bacterial genera described in Brock Biology of Microorganisms Eighth Edition on pages A-8, A-9, A10 and A11.

Microorganisms which are particularly preferred are those able to infect plants and thus transfer the constructs of the invention. Preferred microorganisms are those of the genus Agrobacterium and especially of the species Agrobacterium tumefaciens. Particularly preferred microorganisms are those able to produce toxins (e.g. botulinum toxin), pigments (e.g. carotenoids or flavonoids), antibiotics (e.g. penicillin), phenylpropanoids (e.g. tocopherol), polyunsaturated fatty acids (e.g. arachidonic acid) or vitamins (e.g. vitamin B12).

Preferred yeasts are Candida, Saccharomyces, Hansenula or Pichia.

Preferred fungi are Aspergillus, Trichoderma, Ashbya, Neurospora, Fusarium, Beauveria or further fungi described in Indian Chem Engr. Section B. Vol 37, No. 1,2 (1995) on page 15, table 6.

Host or starting organisms preferred as transgenic organisms are in particular plant organisms.

“Plant organism of cells derived therefrom” means in general every cell, tissue, part or propagation material (such as seeds or fruits) of an organism capable of photosynthesis. Included for the purposes of the invention are all genera and species of higher and lower plants of the plant kingdom. Annual, perennial, monocotyledonous and dicotyledonous plants are preferred.

“Plant” means for the purposes of the invention all genera and species of higher and lower plants of the plant kingdom. The term includes the mature plants, seeds, shoots and seedlings, and parts derived therefrom, propagation material (for example tubers, seeds or fruits), plant organs, tissues, protoplasts, callus and other cultures, for example cell or callus cultures, and all other types of groupings of plant cells to functional or structural units. Mature plants means plants at any stage of development beyond seedling. Seedling means a young, immature plant at an early stage of development.

Plant organisms for the purposes of the invention are additionally further photosynthetically active organisms such as, for example, algae, cyanobacteria and mosses. Preferred algae are green algae, such as, for example, algae of the genus Haematococcus, Phaedactylum tricornatum, Volvox or Dunaliella. Synechocystis, Chlamydomonas and Scenedesmus are particularly preferred.

Particularly preferred for the purposes of the method of the invention are plant organisms selected from the group of flowering plants (Phylum Anthophyta “angiosperms”). All annual and perennial, monocotyledonous and dicotyledonous plants are included. The plant is preferably selected from the following plant families: Amaranthaceae, Asteraceae, Brassicaceae, Caryophyllaceae, Chenopodiaceae, Compositae, Cruciferae, Cucurbitaceae, Labiatae, Leguminosae, Papilionoideae, Liliaceae, Linaceae, Malvaceae, Rosaceae, Rubiaceae, Saxifragaceae, Scrophulariaceae, Solanaceae, Sterculiaceae, Tetragoniaceae, Theaceae and Umbelliferae.

The invention is very particularly preferably applied to dicotyledonous plant organisms. Preferred dicotyledonous plants are in particular selected from the dicotyledonous crop plants such as, for example the following

1) Category: Dicotyledonae (dicotyledons). Preferred families:
Aceraceae (maples)
Cactaceae (cacti)
Rosaceae (roses, apples, almonds, strawberries)
Salicaceae (willows)
Asteraceae (compositae) especially the genus Lactuca, very especially the species sativa (lettuce), and sunflower, dandelion, Tagetes or Calendula and many others,
Cruciferae (Brassicaceae), especially the genus Brassica, very especially the species napus (oilseed rape), campestris (beet), oleracea (e.g. cabbage, cauliflower or broccoli and other brassica species); and of the genus Arabidopsis, very especially the species thaliana, and cress, radish, canola and many others,
Cucurbitaceae such as melon, pumpkin, cucumber or zucchini and many others,
Leguminosae (Fabaceae) especially the genus Glycine, very especially the species max (soybean), soya and alfalfa, pea, beans, lupin or peanut and many others,
Malvaceae, especially mallow, cotton, edible marshmallow, hibiscus and many others,
Rubiaceae, preferably of the subclass Lamiidae such as, for example, Coffea arabica or Coffea liberica (coffee bush) and many others,
Solanaceae, especially the genus Lycopersicon, very especially the species esculentum (tomato) and the genus Solanum, very especially the species tuberosum (potato) and melongena (eggplant) and the genus Capsicum, very especially the species annuum (paprika), and tobacco, petunia and many others,
Sterculiaceae, preferably of the subclass Dilleniidae such as, for example, Theobroma cacao (cocoa plant) and many others,
Theaceae, preferably of the subclass Dilleniidae such as, for example, Camellia sinensis or Thea sinensis (tea bush) and many others,
Umbelliferae (Apiaceae), especially the genus Daucus (very especially the species carota (carrot)), Apium (very especially the species graveolens dulce (celeriac)), and parsley and many others; and flax, hemp, spinach, carrot, sugarbeet and the various tree, nut and vine species, especially bannana and kiwi fruit.

However, in addition, monocotyledonous plants are also suitable. These are preferably selected from the monocotylendonous crop plants such as, for example the families
Arecaceae (palms)
Bromeliaceae (pineapple, spanish moss)
Cyperaceae (sedges)
Liliaceae (lilies, tulips, hyacinths, onions, garlic)
Orchidaceae (orchids)
Poaceae (grasses, bamboos, corn, sugarcane, wheat)
Iridaceae (buckwheat, gladioli, crocuses)

Very particular preference is given to Gramineae such as rice, corn, wheat or other cereal species such as barley, millet, rye, triticale or oats, and the sugarcane, and all species of grasses.

Within the framework of the expression cassette of the invention, expression of a particular nucleic acid may, through a promoter having specificity for the flower of plants, lead to the formation of sense RNA, antisense RNA or double-stranded RNA in the form of an inverted repeat (dsRNAi). The sense RNA can subsequently be translated into particular polypeptides. It is possible with the antisense RNA and dsRNAi to down regulate the expression of particular genes.

The method of gene regulation by means of double-stranded RNA (“double-stranded RNA interference”; dsRNAi) has been described in animal and plant organisms many times (e.g. Matzke M A et al. (2000) Plant Mol Biol 43:401-415; Fire A et al (1998) Nature 391:806-811; WO 99/32619; WO 99/53050; WO 00/68374; WO 00/44914; WO 00/44895; WO 00/49035; WO 00/63364). Express reference is made to the processes and methods described in the citations indicated.

The specificity of the expression constructs and vectors of the invention for flowers of plants is particularly advantageous. The flower has the function in attracting beneficial insects through incorporation of pigments or synthesis of volatile chemicals.

The natural defense mechanisms of the plant, for example against pathogens, are often inadequate. Introduction of foreign genes from plants, animals or microbial sources may enhance the defenses. Examples are protection against insect damage to tobacco through expression of the Bacillus thuringiensis endotoxin (Vaeck et al. (1987) Nature 328:33-37) or protection of tobacco from fungal attack through expression of a chitinase from beans (Broglie et al. (1991) Science 254:1194-1197).

Cold spells during the flowering period lead to considerable crop losses every year. Targeted expression of protective proteins specifically in the flowering period may provide protection.

For such genetic engineering approaches to be highly efficient it is advantageous for there to be concentrated expression of the appropriate nucleic acid sequence to be expressed transgenically in particular in the petals of the flower. Constitutive expression in the whole plant may make the effect problematic, for example through dilution, or impair the growth of the plant or the quality of the plant product. In addition, there may through constitutive expression be increased switching-off of the transgene (“gene silencing”).

Promoters having specificity for the flower are advantageous in this connection. The skilled worker is aware of a large number of proteins whose recombinant expression in the flower is advantageous. The skilled worker is also aware of a large number of genes through which advantageous effects can likewise be achieved through repression or switching-off thereof by means of expression of a corresponding antisense RNA. Non-restrictive examples of advantageous effects which may be mentioned are: achieving resistance to abiotic stress factors (heat, cold, aridity, increased moisture, environmental toxins, UV radiation) and biotic stress factors (pathogens, viruses, insects and diseases), improving the properties of human and animal foods, improving the growth rate or the yield, achieving a longer or earlier flowing period, altering or enhancing the scent or the coloring of the flowers. Non-restricive examples of the nucleic acid sequences or polypeptides which can be employed in these applications and which may be mentioned are:
1. Improved UV protection of the flowers of plants through alteration of the pigmentation through expression of particular polypeptides such as enzymes or regulators of flavonoid biosynthesis (e.g. chalcone synthases, phenylalanine ammonia-lyases), of DNA repair (e.g. photolyases; Sakamoto A et al.(1998) DNA Seq 9(5-6):335-40), of isoprenoid biosynthesis (e.g. deoxyxylulose-5-phosphate synthases), of IPP synthesis or of carotenoid biosynthesis (e.g. phytoene synthases, phytoene desaturases, lycopene cyclases, hydroxylases or ketolases). Preference is given to nucleic acids which code for the Arabidopsis thaliana chalcone synthase (GenBank Acc. No.: M20308), the Arabidopsis thaliana 6-4 photolyase (GenBank Acc. No.:BAB00748) or the Arabidopsis thaliana blue light photoreceptor/photolyase homolog (PHH1) (GenBank Acc. No.: U62549) or functional equivalents thereof.
2. Improved protection of the flower of plants from abiotic stress factors such as aridity, heat or cold, for example through overexpression of the antifreeze polypeptides (e.g. from Myoxocephalus scorpius; WO 00/00512), of the Arabidopsis thaliana transcription activator CBF1, glutamate dehydrogenases (WO 97/12983, WO 98/11240), a late embryogenesis gene (LEA), for example from barley (WO 97/13843), calcium-dependent protein kinase genes (WO 98/26045), calcineurins (WO 99/05902), farnesyl transferases (WO 99/06580; Pei Z M et al. (1998) Science 282:287-290), ferritin (Deak M et al. (1999) Nature Biotechnology 17:192-196), oxalate oxidase (WO 99/04013; Dunwell J M (1998) Biotechnology and Genetic Engeneering Reviews 15:1-32), DREB1A factor (dehydration response element B 1A; Kasuga M et al. (1999) Nature Biotechnology 17:276-286), genes of mannitol or trehalose synthesis (e.g. trehalose-phosphate synthases; trehalose-phosphate phosphatases, WO 97/42326); or through inhibition of genes such as of trehalase (WO 97/50561). Particular preference is given to nucleic acids which code for the Arabidopsis thaliana transcriptional activator CBF1 (Gen-Bank Acc. No.: U77378) or the antifreeze protein from Myoxocephalus octodecemspinosus (GenBank Acc. No.: AF306348) or functional equivalents thereof.
3. Achieving resistance for example to fungi, insects, nematodes and diseases through targeted secretion or accumulation of certain metabolites or proteins in the flower. Examples which may be mentioned are glucosinolates (nematode defense), chitinases or glucanases and other enzymes which destroy the cell wall of parasites, ribosome-inactivating proteins (RIPs) and other proteins of the plant resistance and stress response, like those induced on injury or microbial attack of plants or chemically by, for example, salicylic acid, jasmonic acid or ethylene, lysozymes from non-plant sources such as, for example, T4 lysozyme or lysozme from various mammals, insecticidal proteins such as Bacillus thuringiensis endotoxin, α-amylase inhibitor or protease inhibitors (cowpea trypsin inhibitor), glucanases, lectins (e.g. phytohemagglutinin, snowdrop lectin, wheatgerm agglutinin), RNAses or ribozymes. Particular preference is given to nucleic acids which code for the chit42 endochitinase from Trichoderma harzianum (GenBank Acc. No.: S78423) or for the N-hydroxylating, multifunctional cytochrome P-450 (CYP79) from Sorghum bicolor (GenBank Acc. No.: U32624) or functional equivalents thereof.
4. Achieving defense against or attraction of insects, for example through increased release of volatile scents or messengers through, for example, enzymes of terpene biosynthesis.
5. Achieving an ability to store in flower tissues which normally contain no storage proteins or lipids, with the aim of increasing the yield of these substances, e.g. by expression of an acetyl-CoA carboxylase or of enzymes for esterification of metabolites. Preference is given to nucleic acids which code for the Medicago sativa acetyl-CoA carboxylase (Accase) (GenBank Acc. No.: L25042) or functional equivalents thereof.
6. Expression of transport proteins which improve the uptake of metabolites, nutrients or water into the flower and thus optimize flower growth, metabolite composition or yield, for example through expression of an amino acid transporter which increases the rate of uptake of amino acids, or of a monosaccharide transporter which promotes the uptake of sugars. Preference is given to nucleic acids which code for the Arabidopsis thaliana cationic amino acid transporter (GenBank Acc. No.: X92657) or for the Arabidopsis thaliana monosaccharide transporter (Gen-Bank Acc. No.: AJ002399) or functional equivalents thereof.
7. Expression of genes which bring about an accumulation of fine chemicals, such as of tocopherols, tocotrienols, phenylpropanoids, isoprenoids or carotenoids, in the flower. Examples which may be mentioned are the deoxyxylulose-5-phosphate synthases, phytoene synthases, lycopene β-cyclases and the β-carotene ketolases. Preference is given to nucleic acids which code for the Haematococcus pluvialis NIES-144 (Acc. No. D45881) ketolase or functional equivalents thereof.
8. Modification of wax ester formation or of the composition of the deposited oligosaccharides to improve protection against environmental effects or to improve digestibility on use in animal or human foods. An example which may be mentioned is overexpression of endo-xyloglucan transferase. Preference is given to nucleic acids which code for the Arabidopsis thaliana endo-xyloglucan transferase (EXGT-Al) (Gen-Bank Acc.

No.:AF163819) or functional equivalents thereof.
9. Expression of genes, DNA binding proteins, dsRNA and antisense constructions for altering the flower morphology, the time of flowering and the flower senescence, and the flower metabolism. Preference is given to constructions which increase the number of petals, e.g. through down regulation of AGAMOUS and its homologous genes (Yanofsky M F et al. (1990) Nature 346:35-39), make the time of flowering earlier, e.g. through down regulation of FLOWERING LOCUS C (FLC) (Tadege M et al. (2001) Plant J 28(5):545-53) or later, e.g. through overexpression of FLC and delay senescence, e.g. through conferring a flower-specific ethylene insensitivity.
10. Generation of sterile plants by preventing pollenation and/or germination by means of the expression of a suitable inhibitor, for example of a toxin, in flowers.
11. Production of nutraceuticals such as, for example,
   a) carotenoids and/or phenylpropanoids e.g. through optimization of the flowers' own metabolic pathways, e.g. through expression of enzymes and regulators of isoprenoid biosynthesis. Preference is given to nucleic acids which code for the Arabidopsis thaliana chalcone synthase (GenBank Acc. No.: M20308), the Arabidopsis thaliana 6-4 photolyase (GenBank Acc. No.:BAB00748) or the Arabidopsis thaliana blue light photoreceptor/photolyase homolog (PHH1) (GenBank Acc. No.: U62549) or functional equivalents thereof. Preference is likewise given to nucleic acids which code for enzymes and regulators of isoprenoid biosynthesis such as the deoxyxylulose-5-phosphate synthases and of carotenoid biosynthesis such as the phytoene synthases, lycopene cyclases and ketolases, such as of tocopherols, tocotrienols, phenylpropanoids, isoprenoids or carotenoids, in the flower. Examples which may be mentioned are the deoxyxylulose-5-phosphate synthases, phytoene synthases, lycopene cyclases and the carotene ketolases. Particular preference is given to nucleic acids which code for the Haematococcus pluvialis, NIES-144 (Acc. No. D45881) ketolase or functional equivalents.
   b) polyunsaturated fatty acids such as, for example, arachidonic acid or EPA (eicosapentaenoic acid) or DHA (docosahexaenoic acid) through expression of fatty acid elongases and/or desaturases or production of proteins having improved nutritional value, such as, for example, having a high content of essential amino acids (e.g. the methionine-rich 2S albumin gene of the Brazil nut). Preference is given to nucleic acids which code for the Bertholletia excelsa methionine-rich 2S albumin (GenBank Acc. No.: AB044391), the Physcomitrella patens Δ6-acyl lipid desaturase (GenBank Acc. No.: AJ222980; Girke et al. (1998) Plant J 15:39-48), the Mortierella alpina Δ6-desaturase (Sakura-dani et al 1999 Gene 238:445-453), the Caenorhabditis elegans Δ5-desaturase (Michaelson et al. (1998) FEBS Letters 439:215-218), the Caenorhabditis elegans Δ5-fatty-acid desaturase (des-5) (GenBank Acc. No.: AF078796), the Mortierella alpina Δ5-desaturase (Michaelson et al. J Biol Chem 273:19055-19059), the Caenorhabditis elegans Δ6-elongase (Beaudoin et al. (2000) Proc Natl. Acad. Sci. 97:6421-6426), the Physcomitrella patens Δ6-elongase (Zank et al. (2000,) Biochemical Society Transactions 28:654-657) or functional equivalents thereof.
12. Production of pharmaceuticals such as, for example, antibodies, vaccines, hormones and/or antibiotics as described, for example, in Hood E E & Jilka J M (1999) Curr Opin Biotechnol 10(4):382-6; Ma J K & Vine N D (1999) CurrTop Microbiol Immunol 236:275-92.

Further examples of advantageous genes are mentioned for example in Dunwell J M (2000) Transgenic approaches to crop improvement. J Exp Bot. 51 Spec No:487-96.

A further aspect of the invention relates to the use of the transgenic organisms of the invention described above, and of the cells, cell cultures, parts—such as, for example, in the case of transgenic plant organisms roots, leaves etc.—and transgenic propagation materials such as seeds or fruits, derived therefrom for producing human or animal foods, pharmaceuticals or fine chemicals.

Preference is further given to a method for the recombinant production of pharmaceuticals or fine chemicals in host organisms, where a host organism is transformed with one of the expression cassettes described above, and this expression cassette comprises one or more structural genes which code for the desired fine chemical, or catalyze the biosynthesis thereof, the transformed host organism is cultivated, and the desired fine chemical is isolated from the cultivation medium. This method can be applied widely to fine chemicals such as enzymes, vitamins, amino acids, sugars, fatty acids, natural and synthetic flavorings, aromatizing substances and colorants. Production of tocopherols and tocotrienols, and carotenoids such as, for example, astaxanthin is particularly preferred. Cultivation of the transformed-host organisms and isolation from the host organisms or from the cultivation medium takes place by methods known to the skilled worker. The production of pharmaceuticals such as, for example, antibodies or vaccines is described in Hood E E & Jilka J M (1999) Curr Opin Biotechnol 10 (4)382-6; Ma J K & Vine N D (1999) Curr Top Microbiol Immunol 236:275-92.

A further aspect of the invention relates to the use of the ε-cyclase promoter sequences of the invention (preferably the sequences shown in SEQ ID NO: 1, 7 or 8) for reducing the amount of protein, amount of mRNA and/or activity of an ε-cyclase.

Thus, when an ε-cyclase activity is reduced by comparison with the wild type, the amount of lycopene converted, or the amount of δ-carotene formed, in a particular time by the ε-cyclase protein is reduced by comparison with the wild type.

“Reducing” or “reduce” is to be interpreted broadly in connection with an ε-cyclase or the amount of protein, amount of mRNA and/or activity, and includes the partial or substantially complete inhibition or blocking, based on various cell-biological mechanisms, of the functionality of an ε-cyclase in a plant cell, plant or a part, tissue, organ, cells or seeds derived therefrom.

A reduction for the purposes of the invention also includes a quantitative reduction in an ε-cyclase as far as substantially complete absence of the ε-cyclase (i.e. undetectability of ε-cyclase activity or immunological undetectability of the ε-cyclase). In this connection, a particular ε-cyclase (or the relevant amount of protein, amount of mRNA and/or activity) in a cell or an organism is reduced preferably by at least 5%, further preferably at least 20%, further preferably at least 50%, further preferably 100%. Reduction means in particular also the complete absence of the ε-cyclase (or of its amount of protein, amount of mRNA and/or activity).

Various strategies for reducing the amount of protein, amount of mRNA and/or activity of the ε-cyclase are included according to the invention. The skilled worker will appreciate that a number of different methods are available for influencing the amount of protein, amount of mRNA and/or activity of an ε-cyclase in the desired way. For example, the reduction can be achieved by introducing at least one double-stranded ribonucleic acid sequence which has at least partial homology with the ε-cyclase promoter sequences of the invention (ε-cyclase promoter dsRNA). An alternative possibility is also to attach expression cassettes ensuring dsRNA expression.

The method of gene regulation by means of double-stranded RNA (“double-stranded RNA interference”; dsRNAi) has been described many times for animal and plant organisms (e.g. Matzke M A et al. (2000) Plant Mol Biol 43:401-415; Fire A. et al (1998) Nature 391:806-811; WO 99/32619; WO 99/53050; WO 00/68374; WO 00/44914; WO 00/44895; WO 00/49035; WO 00/63364). Reference is hereby expressly made to the processes and methods described in the indicated citations. dsRNAi methods are based on the phenomenon of simultaneous introduction of strand and complementary strand of a gene transcript bringing about a highly efficient suppression as the expression of the corresponding gene. The resulting phenotype is very similar to that of a corresponding knock-out mutant (Waterhouse P M et al. (1998) Proc Natl Acad Sci USA 95:13959-64).

“Double-stranded RNA molecule” means for the purposes of the invention preferably one or more ribonucleic acid sequences which are able because of complementary sequences theoretically (e.g. according to the base-pair rules of Watson and Crick) and/or actually (e.g. on the basis of hybridization experiments in vitro and/or in vivo) to form double-stranded RNA structures. The skilled worker is aware that the formation of double-stranded RNA structures represents a dynamic equilibrium. The ratio of double-stranded molecules to corresponding dissociated forms is preferably at least 1 to 10, preferably 1:1, particularly preferably 5:1, most preferably 10:1.

A further aspect of the invention therefore relates to double-stranded RNA molecules (dsRNA molecules) which, on introduction into a plant organism (or a cell, tissue, organ or propagation material derived therefrom), bring about the reduction of at least one ε-cyclase. The double-stranded RNA molecule for reducing the expression of an ε-cyclase (ε-cyclase dsRNA) in this case preferably includes
a) a sense RNA strand including at least one ribonucleotide sequence which is substantially identical to at least part of a nucleic acid sequence coding for the promoter region of an ε-cyclase, and
b) an antisense RNA strand which is substantially—preferably completely—complementary to the RNA sense strand under a).

The promoter region of the ε-cyclase is preferably described by a sequence as shown in SEQ ID NO: 1, 7 or 8.

“Substantially identical” means that the dsRNA sequence may also have insertions, deletions and single point mutations compared with the ε-cyclase promoter target sequence, and nevertheless brings about an efficient reduction of expression. The homology (as defined hereinafter) is preferably at least 75%, preferably at least 80%, very particularly preferably at least 90%, most preferably 100%, between the sense strand of an inhibitory dsRNA and at least part of the nucleic acid sequence coding for an ε-cyclase promoter (or between the antisense strand and the complementary strand of a nucleic acid sequence coding for an ε-cyclase promoter). The skilled worker moreover is aware that, in a comparison of homology between RNA and DNA, the bases uracil and thymine are to be regarded as equivalent.

A 100% sequence identity between dsRNA and an ε-cyclase promoter is not absolutely necessary for bringing about an efficient reduction of ε-cyclase expression. Accordingly, there is the advantage that the method is tolerant to sequence differences like those which may be present owing to genetic mutations, polymorphisms or evolutionary divergences.

The length of the partial segment is at least 10 bases, preferably at least 25 bases, particularly preferably at least 50 bases, very particularly preferably at least 100 bases, most preferably at least 200 bases or at least 300 bases.

It is alternatively possible for a “substantially identical” dsRNA also to be defined as nucleic acid sequence which is able to hybridize with part of an ε-cyclase gene or promoter sequence (e.g. in 400 mM NaCl, 40 μM PIPES pH 6.4, 1 μM EDTA at 50° C. or 70° C. for 12 to 16 h).

“Substantially complementary” means that the antisense RNA strand may also have insertions, deletions and single point mutations by comparison with the complement of the sense RNA strand. The homology is preferably at least 80%, preferably at least 90%, very particularly preferably at least 95%, most preferably 100%, between the antisense RNA strand and the complement of the sense RNA strand.

“Part of a nucleic acid sequence coding for an ε-cyclase promoter” means fragments of a nucleic acid sequence coding for an ε-cyclase promoter, preferably the promoter sequences as shown in SEQ ID NO: 1, 2 or 3 or functional equivalents thereof. In this connection, the fragments preferably have a sequence length of at least 20 bases, preferably at least 50 bases, particularly preferably at least 100 bases, very particularly preferably at least 200 bases, most preferably at least 500 bases.

It is particularly advantageous to use the ε-cyclase promoter region to reduce the ε-cyclase activity because only low homologies with other genes are present here, and thus the reduction can be highly specific without effecting the expression of other genes.

The dsRNA can consist of one or more strands of polyribonucleotides. It is, of course, also possible to achieve the same purpose by introducing a plurality of individual dsRNA molecules, each of which include one of the ribonucleotide sequence segments defined above, into the cell or the organism. The double-stranded dsRNA structure can be formed starting from two complementary separate RNA strands or—preferably—starting from a single self-complementary RNA strand. In this case, sense RNA strand and antisense RNA strand are preferably connected together covalently in the form of an inverted repeat.

In a preferred embodiment, a further aspect of the invention includes ribonucleic acid molecules including
a) at least one ribonucleotide sequence which is substantially identical to at least one part of a nucleic acid sequence coding for the promoter region of an ε-cyclase, and
b) at least one further ribonucleotide sequence which is substantially complementary to at least one part of the ribonucleotide sequence under a),
   where a) and b) are connected together covalently, and further functional elements may be located where appropriate between a) and b).

The promoter region of the ε-cyclase is preferably described by a sequence as shown in SEQ ID NO: 1, 7 or 8.

As described, for example, in WO 99/53050, the dsRNA may also include a hairpin structure through connection of sense and antisense strands by a connecting sequence (“linker”; for example an intron). The self-complementary dsRNA structures are preferred, because they require merely the expression of one RNA sequence and include the complementary RNA strands always in an equimolar ratio. The connecting sequence is preferably an intron (e.g. an intron of the potato ST-LS1 gene; Vancanneyt G F et al. (1990) Mol Gen Genet 220(2):245-250).

If the two strands of the dsRNA are to be put together in a cell or plant, this can take place in the following way, for example:
a) transformation of the cell or plant with a vector which includes both expression cassettes,
b) cotransformation of the cell or plant with two vectors, where one includes the expression cassettes with the sense strand the other includes the expression cassettes with the antisense strand,
c) crossing of two individual plant lines, where one includes the expression cassettes with the sense strand and the other includes the expression cassettes with the antisense strand.

Formation of the RNA duplex can be initiated either outside the cell or inside it.

The dsRNA can be synthesized either in vivo or in vitro. For this purpose it is possible to put a DNA sequence coding for a dsRNA into an expression cassette under the control of at least one genetic control element (such as, for example, a promoter). Polyadenylation is unnecessary, nor need any elements be present to initiate translation. The expression cassette for the ε-cyclase promoter dsRNA is preferably contained on the expression vector. The invention includes corresponding expression vectors.

In a particular preferred embodiment, expression of the dsRNA takes place starting from an expression construct under the functional control of a flower-specific promoter. The promoter employed in this connection is preferably not the ε-cyclase promoter from which the dsRNA has been derived. However, it is very possible for it to be an ε-cyclase promoter of a different species. Thus, for example, the sunflower ε-cyclase promoter could be used to express the dsRNA derived from the Tagetes erecta ε-cyclase promoter. However, expression of the dsRNA derived from an ε-cyclase promoter is preferably under the control of a promoter which is not an ε-cyclase promoter, particularly preferably under the control of the Cucumis sativus CHRC promoter (SEQ ID NO: 81) or of the AP3P promoter (SEQ ID NO: 77) or of a functionally equivalent part thereof.

The expression cassettes coding for the antisense and/or the sense strand of an ε-cyclase dsRNA or for the self-complementary strand of the dsRNA are for this purposes preferably inserted into a transformation vector and introduced into the plant cell using the methods described below. Stable insertion into the genome is advantageous for the method of the invention.

The dsRNA can be introduced in an amount which makes at least one copy possible per cell. Larger amounts (e.g. at least 5, 10, 100, 500 or 1000 copies per cell) may where appropriate bring about a more efficient reduction.

The invention also includes methods for producing ketocarotenoids, where the amount of mRNA and/or activity of at least one ε-cyclase is reduced by introducing at least one of the double-stranded RNA sequences or ribonucleic acid sequences of the invention or an expression cassette or expression cassettes ensuring expression thereof.

Ketocarotenoids means carotenoids which comprise at least one keto group, such as, for example, astaxanthin, canthaxanthin, echinenone, 3-hydroxyechinenone, 3′-hydroxyechinenone, adonirubin and adonixanthin.

Sequences

1. SEQ ID NO: 1 nucleic acid sequence coding for the Tagetes erecta ε-cyclase promoter
2. SEQ ID NO: 2 nucleic acid sequence coding for the Tagetes erecta ε-cyclase promoter including the ε-cyclase 5′-untranslated region
3. SEQ ID NO: 3 nucleic acid sequence coding for the Tagetes erecta ε-cyclase promoter including 5′-untranslated region and region coding for the transit peptide
4. SEQ ID NO: 4 amino acid sequence of the putative Tagetes erecta ε-cyclase transit peptide
5. SEQ ID NO: 5 nucleic acid sequence coding for the Tagetes erecta ε-cyclase promoter including the ε-cyclase 5′-untranslated region flanked by restriction cleavage sites for cloning
6. SEQ ID NO: 6 nucleic acid sequence coding for the Tagetes erecta ε-cyclase promoter including 5′-untranslated region and region coding for the transit peptide flanked by restriction cleavage sites for cloning
7. SEQ ID NO: 7 nucleic acid sequence coding for the Arabidopsis thaliana ε-cyclase promoter including the ε-cyclase 5′-untranslated region
8. SEQ ID NO: 8 nucleic acid sequence coding for the Oryza sativa ε-cyclase promoter including the ε-cyclase 5′-untranslated region
9. SEQ ID NO: 9 nucleic acid sequence coding for a Tagetes erecta ε-cyclase
10. SEQ ID NO: 10 amino acid sequence of a Tagetes erecta ε-cyclase
11. SEQ ID NO: 11 nucleic acid sequence coding for a Tagetes erecta ε-cyclase
12. SEQ ID NO: 12 amino acid sequence of a Tagetes erecta ε-cyclase
13. SEQ ID NO: 13 nucleic acid sequence coding for an Arabidopsis thaliana ε-cyclase
14. SEQ ID NO: 14 amino acid sequence of an Arabidopsis thaliana ε-cyclase
15. SEQ ID NO: 15 nucleic acid sequence coding for a rice ε-cyclase
16. SEQ ID NO: 16 amino acid sequence of a rice ε-cyclase
17.-22 SEQ ID NO: 17 to 22: sequence motifs for ε-cyclase proteins
23. SEQ ID NO: 23 nucleic acid sequence coding for a Lactuca sativa ε-cyclase (homologous sequence H1)
24. SEQ ID NO: 24 amino acid sequence of a Lactuca sativa ε-cyclase (homologous sequence H1)
25. SEQ ID NO: 25 nucleic acid sequence coding for an Adonis palaestina ε-cyclase (homologous sequence H2)
26. SEQ ID NO: 26 amino acid sequence of an Adonis palaestina ε-cyclase (homologous sequence H2)
27. SEQ ID NO: 27 nucleic acid sequence coding for an Adonis palaestina ε-cyclase (homologous sequence H3)
28. SEQ ID NO: 28 amino acid sequence of an Adonis palaestina ε-cyclase (homologous sequence H3)
29. SEQ ID NO: 29 nucleic acid sequence coding for an Arabidopsis thaliana ε-cyclase (homologous sequence H4)
30. SEQ ID NO: 30 amino acid sequence of an Arabidopsis thaliana ε-cyclase (homologous sequence H4)
31. SEQ ID NO: 31 nucleic acid sequence coding for a Citrus X paradisi ε-cyclase (homologous sequence H5)
32. SEQ ID NO: 32 amino acid sequence of Citrus X paradisi ε-cyclase (homologous sequence H5)
33. SEQ ID NO: 33 nucleic acid sequence coding for a Citrus X paradisi ε-cyclase (homologous sequence H6)
34. SEQ ID NO: 34 amino acid sequence of Citrus X paradisi ε-cyclase (homologous sequence H6)
35. SEQ ID NO: 35 nucleic acid sequence coding for a Citrus sinensis ε-cyclase (homologous sequence H7)
36. SEQ ID NO: 36 amino acid sequence of a Citrus sinensis ε-cyclase (homologous sequence H7)
37. SEQ ID NO: 37 nucleic acid sequence coding for a Spinacea oleracea ε-cyclase (homologous sequence H8)
38. SEQ ID NO: 38 amino acid sequence of a Spinacea oleracea ε-cyclase (homologous sequence H8)
39. SEQ ID NO: 39 nucleic acid sequence coding for a Solanum tuberosum ε-cyclase (homologous sequence H9)
40. SEQ ID NO: 40 amino acid sequence of a Solanum tuberosum ε-cyclase (homologous sequence H9)
41. SEQ ID NO: 41 nucleic acid sequence coding for a Daucus carota ε-cyclase (homologous sequence H10)
42. SEQ ID NO: 42 amino acid sequence of a Daucus carota ε-cyclase (homologous sequence H10)
43. SEQ ID NO: 43 nucleic acid sequence coding for a Daucus carota ε-cyclase (homologous sequence H11)
44. SEQ ID NO: 44 amino acid sequence of a Daucus carota ε-cyclase (homologous sequence H11)
45. SEQ ID NO: 45 nucleic acid sequence coding for a tomato ε-cyclase (homologous sequence H12)
46. SEQ ID NO: 46 -amino acid sequence of a tomato ε-cyclase (homologous sequence H12)
47. SEQ ID NO: 47 nucleic acid sequence coding for ε-cyclase-specific probe (gecyc1; 510 bp)
48. SEQ ID NO: 48 oligonucleotide primer PR16 5′-ggcacgaggcaaagcaaagg-3′
49. SEQ ID NO: 49 oligonucleotide primer PR22 5′-cgataagtgcgacattcaagc-3′
50. SEQ ID NO: 50 nucleic acid sequence including part of the Tagetes erecta ε-cyclase promoter obtained by iPCR
51. SEQ ID NO: 51 nucleic acid sequence including part of the Tagetes erecta ε-cyclase promoter obtained by TAIL PCR
52. SEQ ID NO: 52 oligonucleotide primer PR50 5′-cgccttgtatctgtttggattgg-3′
53. SEQ ID NO: 53 oligonucleotide primer PR51 5′-ctaacaatcaatgagtatgagagc-3′
54. SEQ ID NO: 54 oligonucleotide primer PR60 5′-agagcaaggccagcaggaccacaacc-3′
55. SEQ ID NO: 55 oligonucleotide primer PR61 5′-ccttgggagcttttgggataggctag-3′
56. SEQ ID NO: 56 oligonucleotide primer PR63 5′-tcacgccttgtatctgtttggattgg-3′
57. SEQ ID NO: 57 oligonucleotide primer from the set of AD1 primers as was found in the amplicon 5′-gtcgagtatggagtt-3′
58. SEQ ID NO: 58 nucleic acid sequence encoding iPCR fragment (734 bp) from pTA-ecycP
59. SEQ ID NO: 59 oligonucleotide primer OL1 5′-ctcgagagtaaaatcgttagttatg-3′
60. SEQ ID NO: 60 oligonucleotide primer OL2 5′-ccatggccattgattgttagtaatgattc-3′
61. SEQ ID NO: 61 oligonucleotide primer OL3 5′-ccatggtaatttgcttcgtgtatctgatg-3′
62. SEQ ID NO: 62 oligonucleotide primer OL4 5′-ccatggcgctagcagcgacagtaatg-3′
63. SEQ ID NO: 63 oligonucleotide primer OL5 5′-gatatccggtgtgagggaactag-3′
64. SEQ ID NO: 64 oligonucleotide primer PR1 5′-gcaagctcgacagctacaaacc-3′
65. SEQ ID NO: 65 oligonucleotide primer PR2 5′-gaagcatgcagctagcagcgacag-3′
66. SEQ ID NO: 66 nucleic acid sequence coding for Ketolase-35S terminator construct
67. SEQ ID NO: 67 oligonucleotide primer PR7 5′-gagctcactc actgatttcc attgcttg-3′
68. SEQ ID NO: 68 oligonucleotide primer PR8 5-cgccgttaagtcgatgtccgttgatttaaacagtgtc-3′
69. SEQ ID NO: 69 oligonucleotide primer PR9 5′-atcaacggac atcgacttaa cggcgtttgt aaac-3′
70. SEQ ID NO: 70 oligonucleotide primer PR10 5′-taagcttttt gttgaagaga tttgg-3′
71. SEQ ID NO: 71 oligonucleotide primer PR40 5′-gtcgactacg taagtttctg cttctacc-3′
72. SEQ ID NO: 72 oligonucleotide primer PR41 5′-ggatccggtg atacctgcac atcaac-3′
73. SEQ ID NO: 73 oligonucleotide primer PR124 5′-aagcttaccg atagtaaaat cgttagtt-3′
74. SEQ ID NO: 74 oligonucleotide primer PR125 5′-ctcgagctta ccgatagtaa aatcgttagt t-3′
75. SEQ ID NO: 75 oligonucleotide primer PR126 5′-gtcgacaaca acaacaaaca acctttgc-3′
76. SEQ ID NO: 76 oligonucleotide primer PR127 5′-ggatccaaca acaacaaaca acctttgc-3′
77. SEQ ID NO: 77 nucleic acid sequence coding for a modified version (AP3P) of the flower-specific Arabidopsis thaliana promoter AP3
78. SEQ ID NO: 78 nucleic acid sequence coding for PIV2 intron of the potato ST-LS1 gene.
79. SEQ ID NO: 79 nucleic acid sequence coding for the sense strand of the dsRNA directed against the ε-cyclase promoter
80. SEQ ID NO: 80 nucleic acid sequence coding for the antisense strand of the dsRNA directed against the ε-cyclase promoter
81. SEQ ID NO: 81 nucleic acid sequence coding for the Cucumis sativus chromoplast-specific carotenoid-associated protein (CHRC) promoter
82. SEQ ID NO: 82 oligonucleotide primer PRCHRC5 5′-gagctctaca aattagggtt ac-3′
83. SEQ ID NO: 83 oligonucleotide primer PRCHRC3 5′-aagcttatta tttccaaatt ccg-3′

### FIGURES

The general abbreviations used in the following figures have the following meaning:
GUSI-Intron-GUSII: reporter gene (bacterial β-glucuronidase)
Intron: Intron
NosT: nopaline synthase (NOS) terminator sequence
RB/LB: right or left T-DNA border
35-T: 35S CaMV terminator
NptII: canamycin resistence
NosP: nopaline synthase (NOS) promoter sequence
aadA: bacterial spectinomycin resistance
colE1: origin of replication

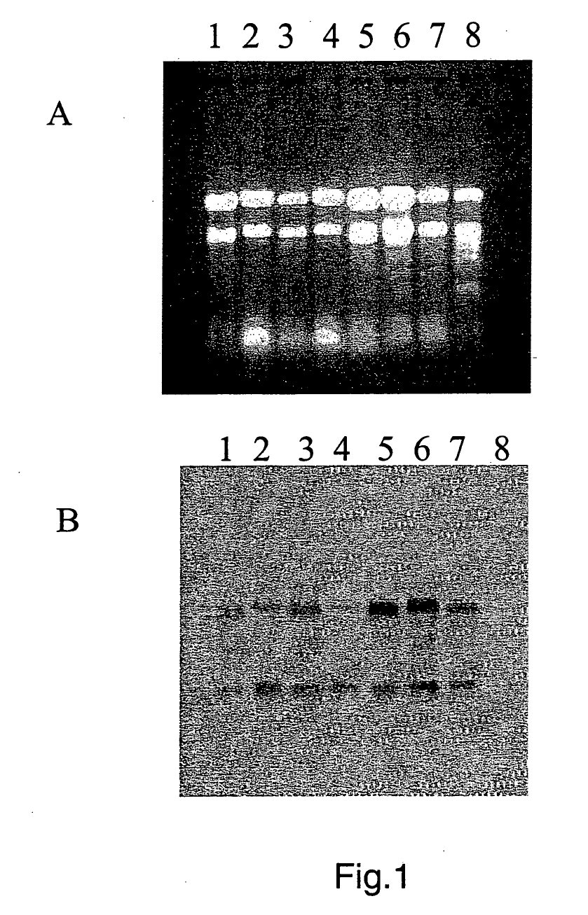

1. FIG. 1: analysis of the ε-cyclase transcript level total RNA isolated from leaves (L) and flower stages (1-7) of Tagetes erecta by means of RNA gel blotting analysis

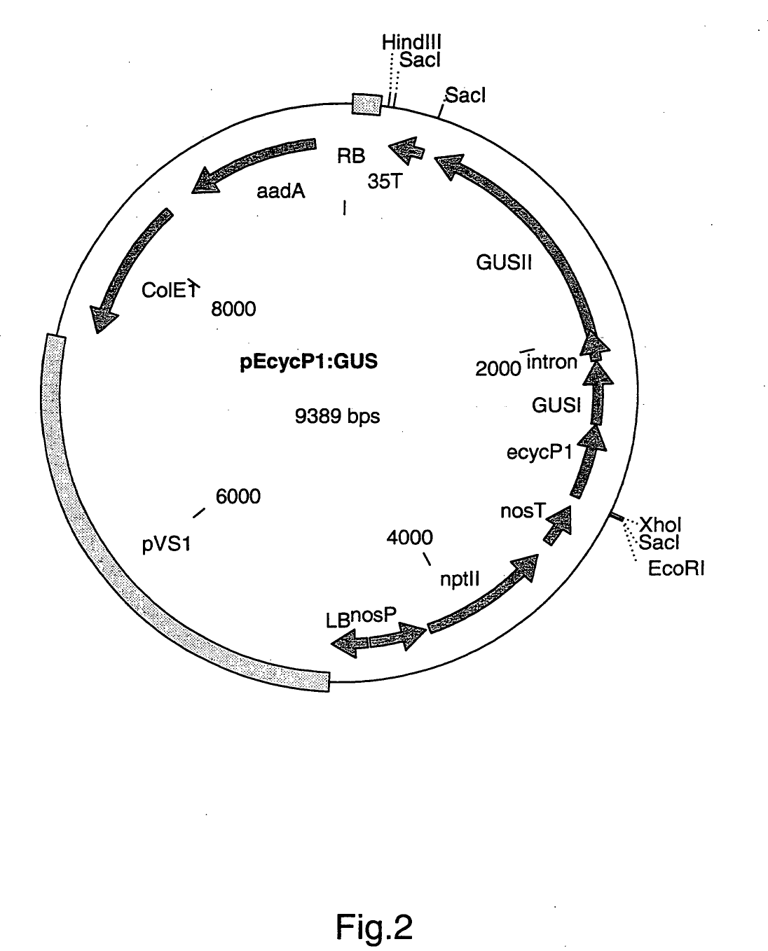

2. FIG. 2: diagrammatic representation of the pEcycP1:GUS vector for flower-specific expression of the β-glucuronidase reporter gene (GUS) under the control of the Tagetes erecta ecycP1 regulatory element (promoter and 5′-untranslated region)
ecycP1: Tagetes erecta ε-cyclase promoter including 5′-untranslated region (SEQ ID NO: 2)

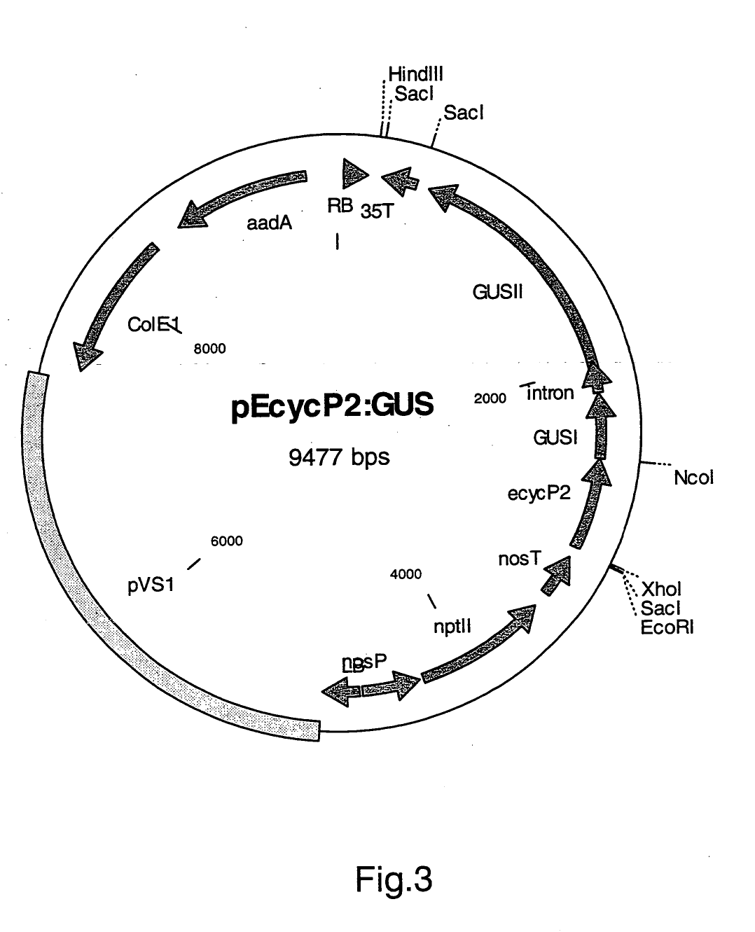

3. FIG. 3: diagrammatic representation of the pEcycP2:GUS vector for flower-specific expression of the β-glucuronidase reporter gene (GUS) under the control of the Tagetes erecta ecycP2 regulatory element (promoter and 5′-untranslated region and transit peptide)
ecycP2: Tagetes erecta ε-cyclase promoter including 5′-untranslated region and transit peptide (SEQ ID NO: 3)

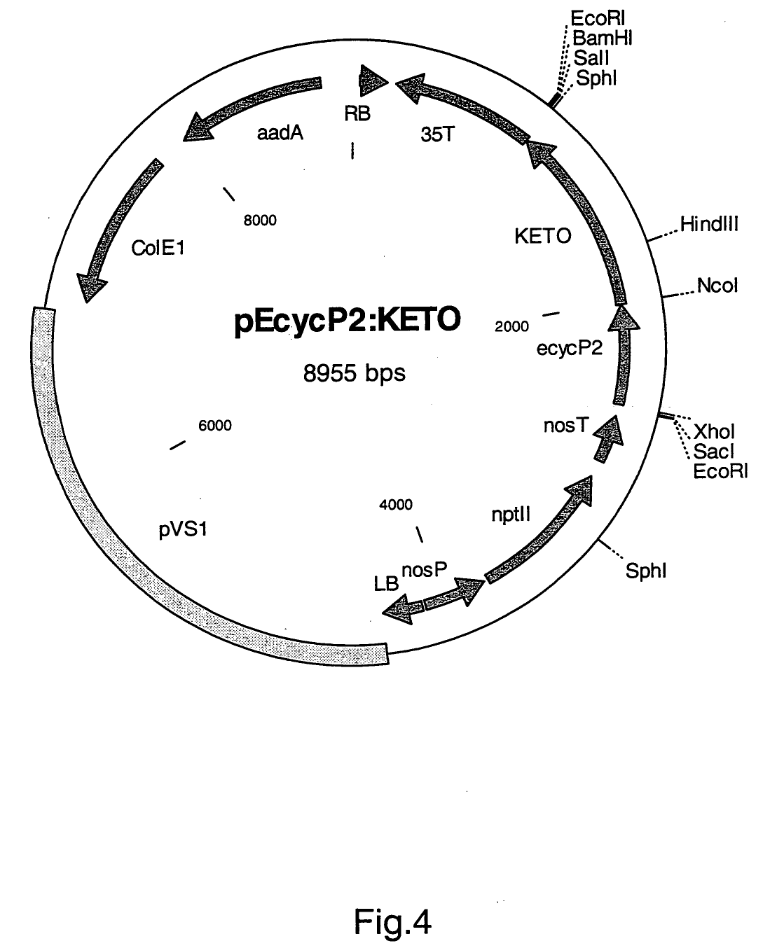

4. FIG. 4: diagrammatic representation of the pEcycP2:KETO vector for flower-specific expression of the Haematococcus pluvialis ketolase (KETO; SEQ ID NO: 66) under the control of the Tagetes erecta ecycP2 regulatory element (promoter and 5′-untranslated region and transit peptide; SEQ ID NO: 3).

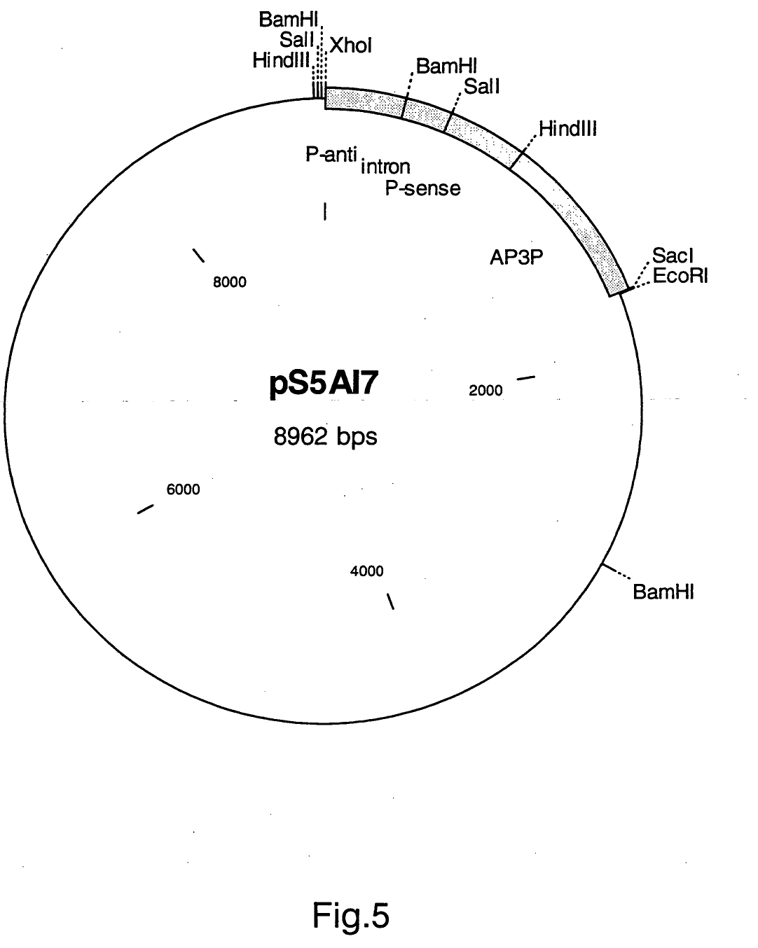

5. FIG. 5: diagrammatic representation of the pS5AI7 vector for flower-specific expression of ε-cyclase promoter specific dsRNA under the control of the AP3P promoter fragment for flower-specific reduction of the ε-cyclase transcript level. AP3P: modified AP3P promoter (777 bp),
P-sense: 358 bp ε-cyclase promoter fragment in sense orientation,
intron: IV2 intron of the potato ST-LS1 gene
P-anti: the 361 bp ε-cyclase promoter fragment in antisense orientation.

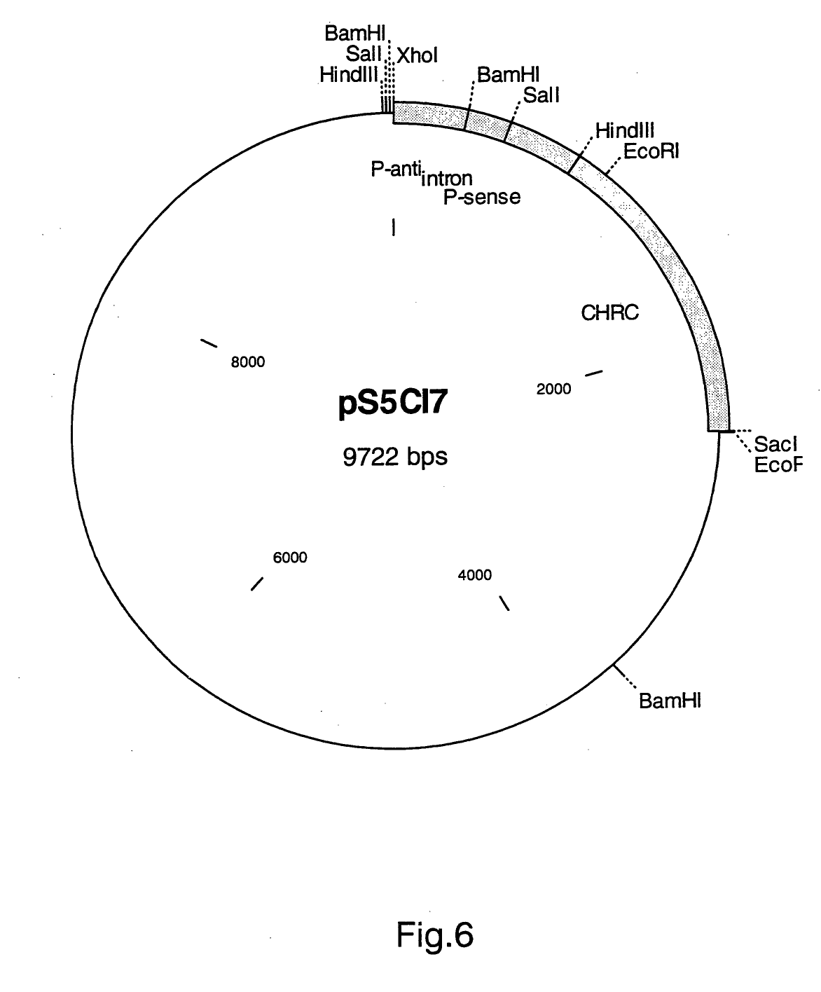

6. FIG. 6: diagrammatic representation of the pS5CI7 vector for flower-specific expression of ε-cyclase promoter specific dsRNA under the control of the CHRC promoter fragment for flower-specific reduction of the ε-cyclase transcript level
CHRC: CHRC promoter (1537 bp),
P-sense: 358 bp ε-cyclase promoter fragment in sense orientation,
intron: IV2 intron of the potato ST-LS1 gene
P-anti: the 361 bp ε-cyclase promoter fragment in antisense orientation.

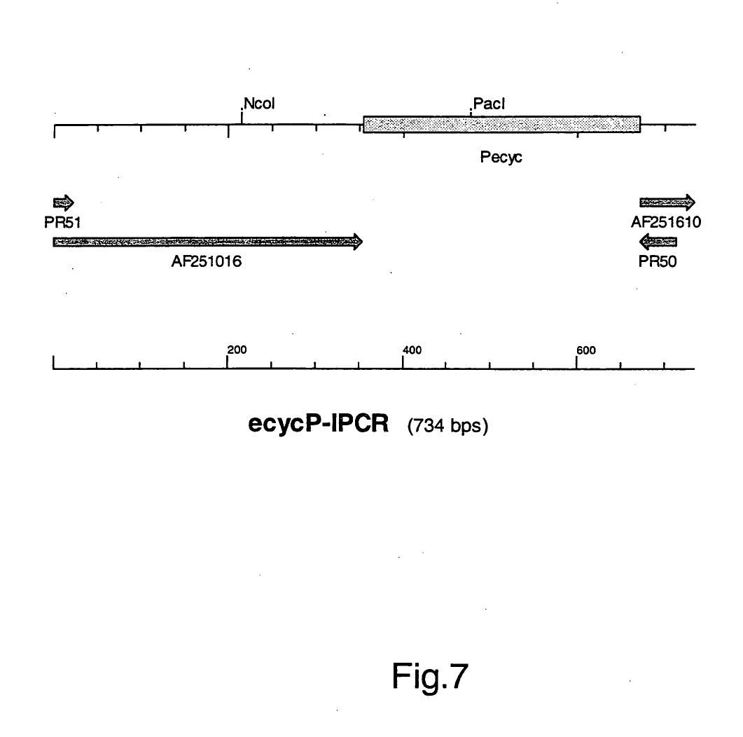

7. FIG. 7: iPCR amplicon comprising the 312 bp fragment of the ε-cyclase promoter

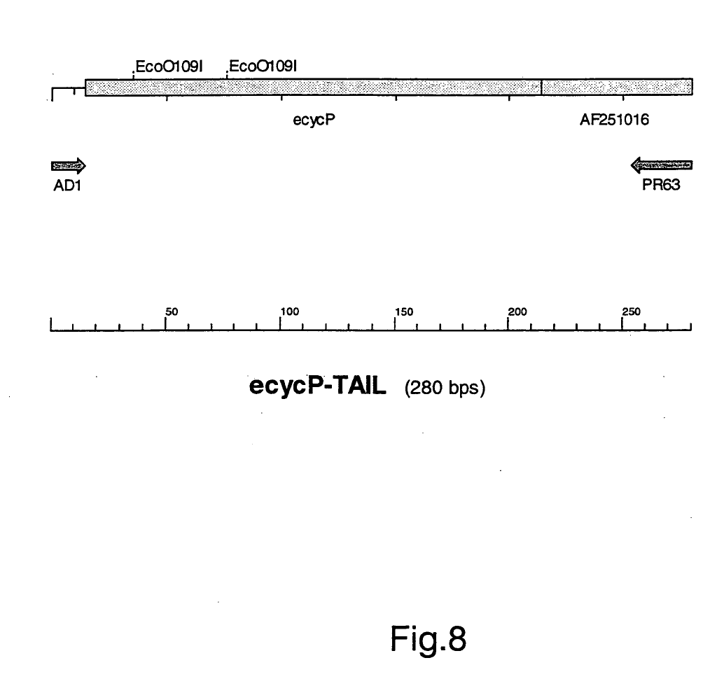

8. FIG. 8: TAIL PCR amplicon comprising the 199 bp fragment of the ε-cyclase promoter

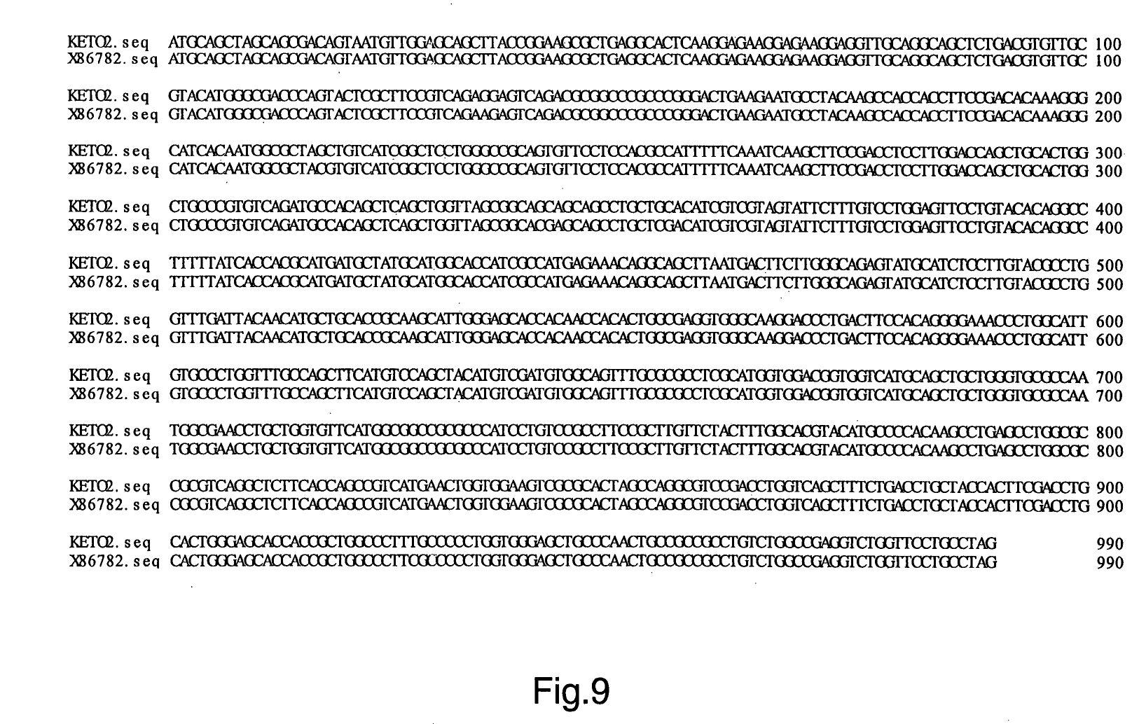

9. FIG. 9: nucleotide sequence comparison between the published sequence of the Haematococcus pluvialis ketolase (GenBank Acc. No.: X86782) and the sequence provided within the scope of the invention (cf. example 3).

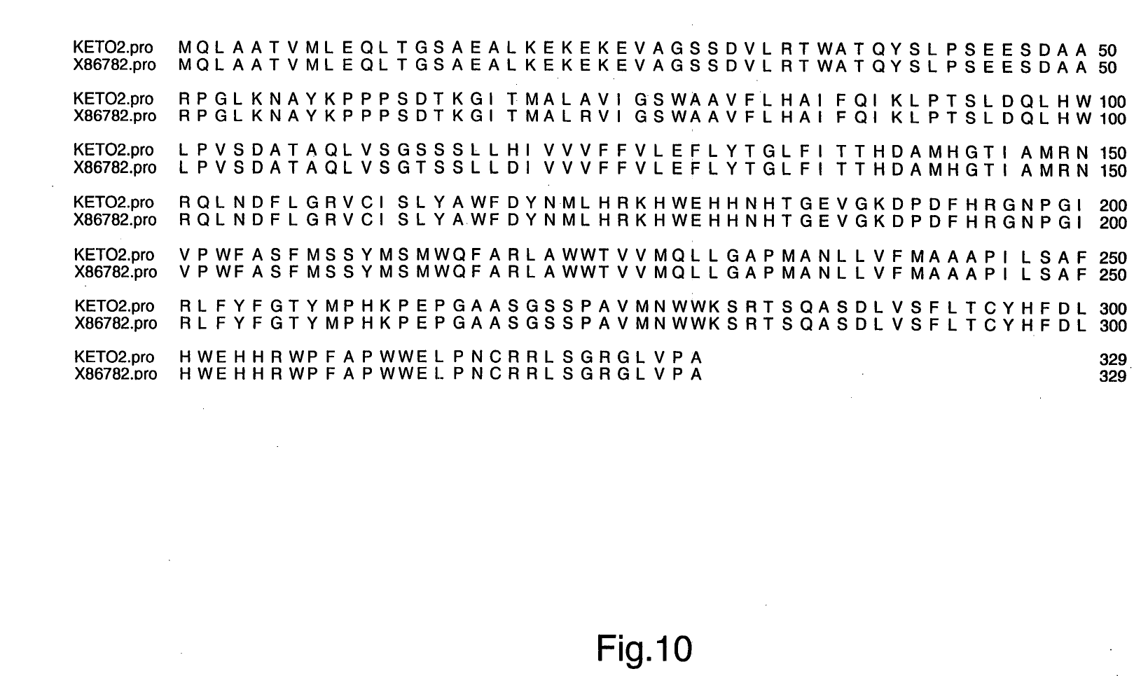

10. FIG. 10: protein sequence comparison between the published sequence of the Haematococcus pluvialis ketolase (GenBank Acc. No.: X86782) and the sequence provided within the scope of the invention (cf. example 3).

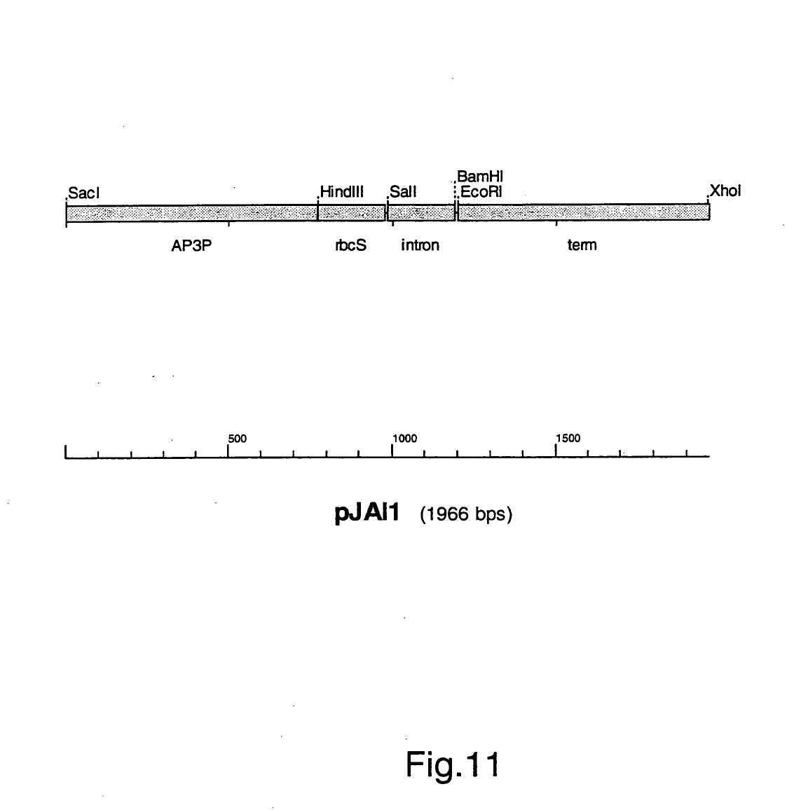

11. FIG. 11: cloning cassette for producing inverted repeat expression cassettes for flower-specific expression of ε-cyclase dsRNAs.
AP3P: modified AP3P promoter (777 bp),
rbcs: pea rbcs transit peptide (206 bp),
intron: PIV2 intron of the ST-LS1 gene (SEQ ID NO: 78)
term: CaMV 35S polyadenylation signal (762 bp).

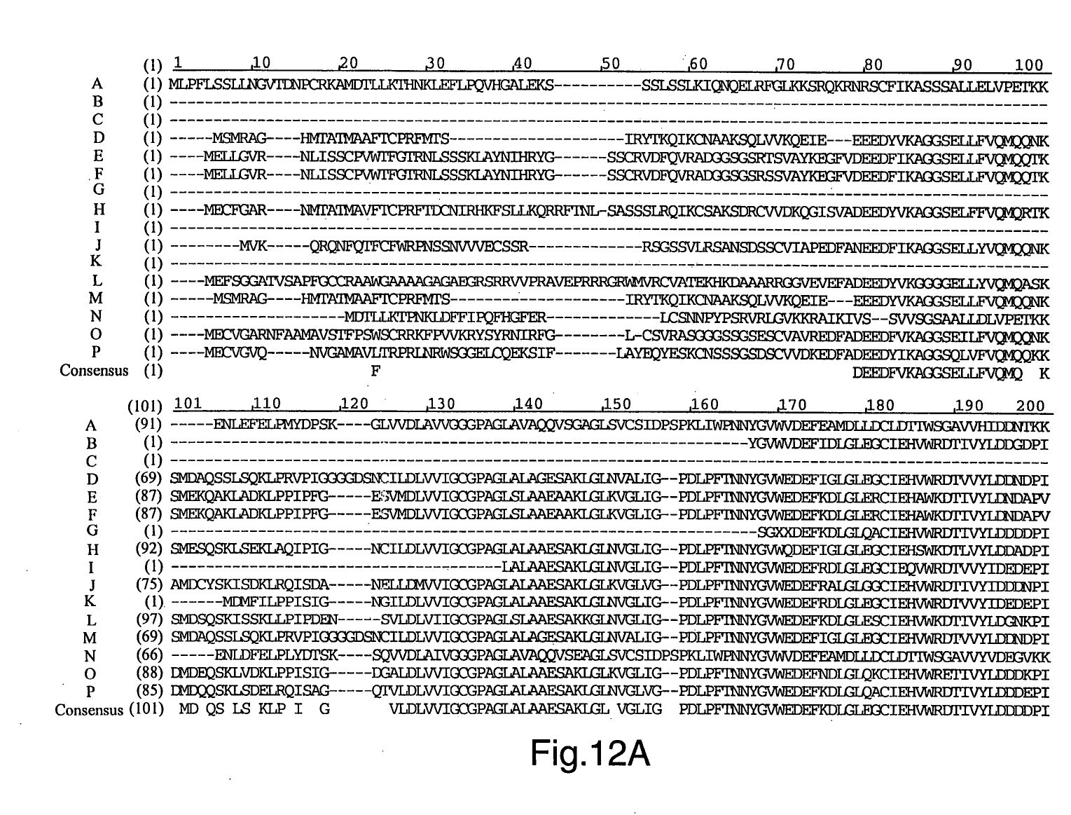

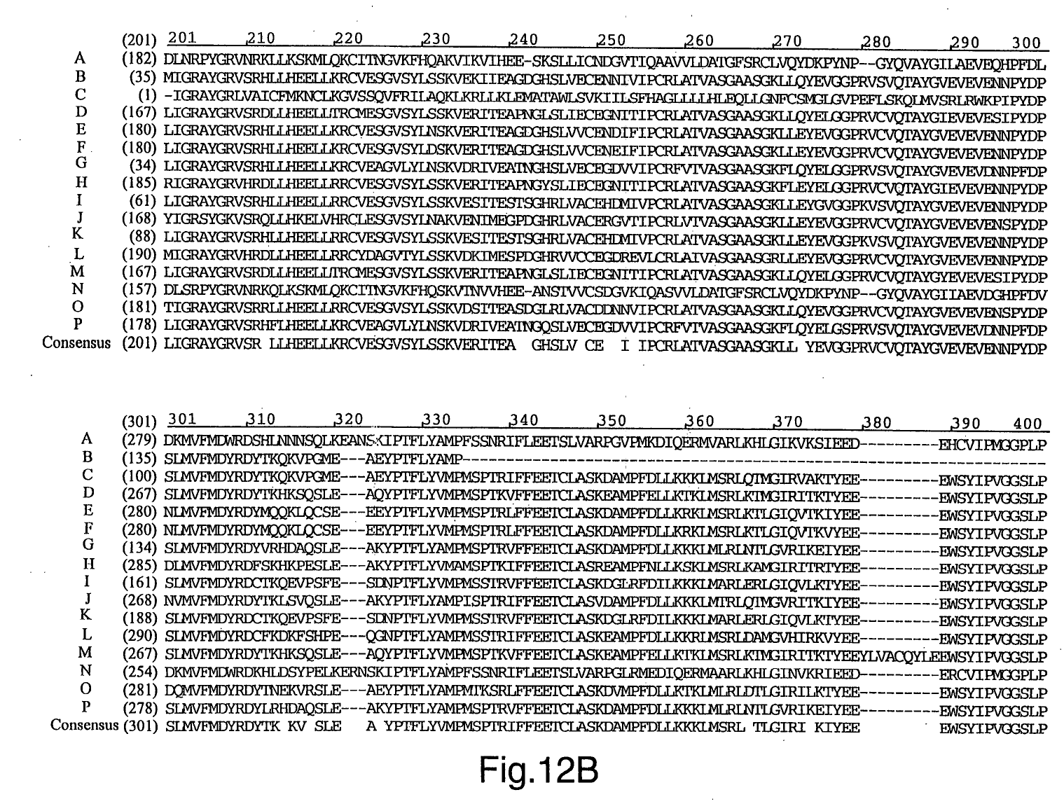

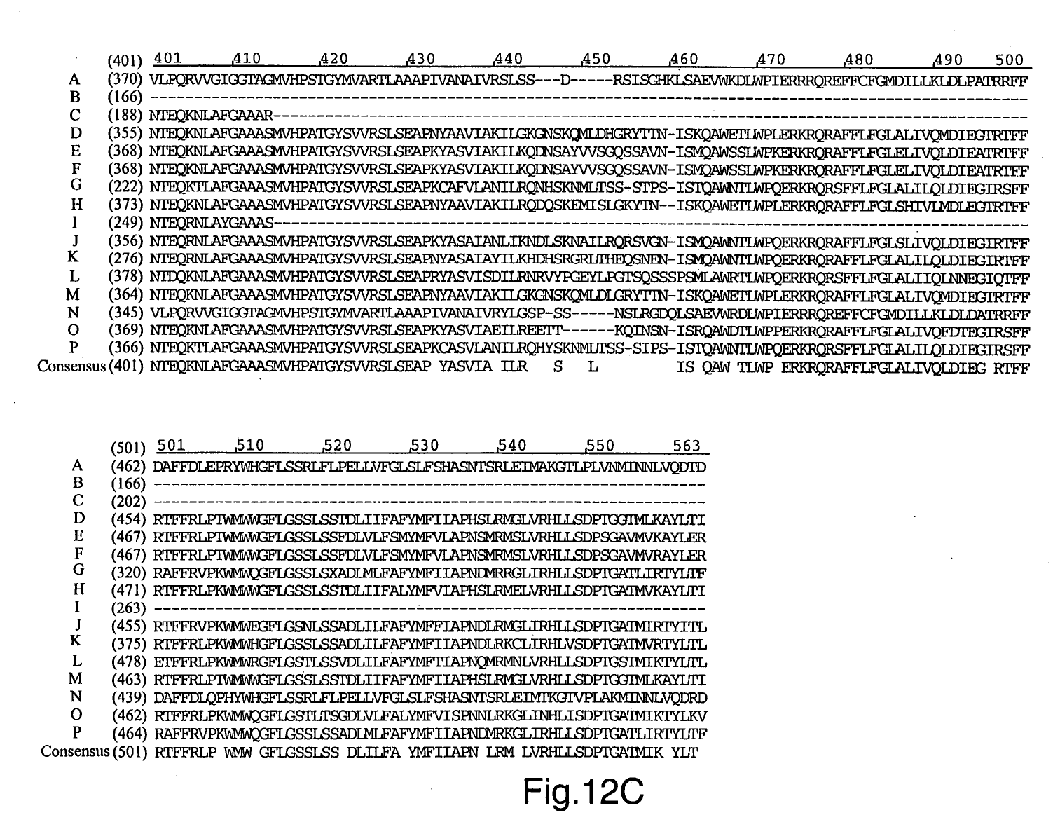

12. FIG. 12A-C: sequence comparison of various plant ε-cyclases.
A: GenBank Acc. No.: AF152246 (524) Citrus x paradisi “lycopene cyclase”
B: GenBank Acc. No.: AF212130 (165) Daucus carota partial ecyclase sequence
C: GenBank Acc. No.: AF229684 (201) Daucus carota partial ecyclase sequence
D: GenBank Acc. No.: AF251016 (516) Tagetes erecta ecyclase
E: GenBank Acc. No.: AF321535 (529) Adonis palaestina ecyclase
F: GenBank Acc. No.: AF321536 (529) Adonis palaestina ecyclase
G: GenBank Acc. No.: AF321537 (382) Solanum tuberosum partial ecyclase sequence
H: GenBank Acc. No.: AF321538 (533) Lactuca sativa ecyclase
I: GenBank Acc. No.: AF450280 (262) Citrus sinensis ecyclase
J: GenBank Acc. No.: AF463497 (517) Spinacea oleracea ecyclase
K: GenBank Acc. No.: AF486650 (437) Citrus x paradisi ecyclase
L: GenBank Acc. No.: AP003332 (540) rice ecyclase
M: GenBank Acc. No.: AY099485 (525) Tagetes erecta ecyclase
N: GenBank Acc. No.: L40176 (501) Arabidopsis “lycopene cyclase”
O: GenBank Acc. No.: NM125085 (524) Arabidopsis ecyclase
P: GenBank Acc. No.: 065837 ecyclase (526) tomato

13. FIG. 13: diagrammatic representation of the inverse PCR (“iPCR”)
For the “iPCR”, genomic DNA of a target organism having the promoter sequence to be isolated is completely digested with a given restriction enzyme, and then the individual fragments are religated, i.e. connected together to form a circular molecule, in a diluted mixture. The large number of resulting circular DNA molecules includes those comprising the known sequence (i.e. the sequence coding for a homologous protein). The circular molecule can be amplified, starting therefrom, by means of PCR using a primer pair in which both primers are able to anneal to the known sequence segment.
Abbreviations: P—promoter sequence; CR—coding region; L—ligation site; PCR—polymerase chain reaction. Arrows represent the binding site of potential oligonucleotide primers in the area of the coding region.

### EXAMPLES

General Methods:

Oligonucleotides can be chemically synthesized for example in a known manner by the phosphoramidite method (Voet & Voet (1995), 2^{nd} edition, Wiley Press New York, pages 896-897). The cloning steps carried out for the purposes of the present invention, such as, for example, restriction cleavages, agarose gel electrophoresis, purification of DNA fragments, transfer of nucleic acids to nitrocellulose and nylon membranes, linkage of DNA fragments, transformation of E. coli cells, culturing of bacteria, replication of phages and sequence analysis of recombinant DNA, are carried out as described in Sambrook et al. (1989) Cold Spring Harbor Laboratory Press; ISBN 0-87969-309-6. Recombinant DNA molecules are sequenced by the method of Sanger (Sanger et al. (1977) Pro Natl Acad Sci USA 74:5463-5467) using an ABI laser fluorescence DNA sequencer.

### Example 1

### Analysis of ε-cyclase RNA Transcript Levels during the Development of Tagetes erecta Flowers

Total RNA from Tagetes erecta leaves and flowers is prepared by harvesting plant tissue, freezing it in liquid nitrogen and powdering in a mortar. 100 mg of the frozen, powdered plant tissue are then transferred into a reaction vessel and taken up in 0.8 ml of Trizol® buffer (LifeTechnologies). The suspension is extracted with 0.2 ml of chloroform. After centrifugation at 12 000 g for 15 minutes, the aqueous supernatant is removed and transferred into a new reaction vessel and extracted with one volume of ethanol. The RNA is precipitated with one volume of isopropanol and washed with 75% ethanol, and the pellet is dissolved in DEPC water (water incubated with 1/1000 volume of diethyl pyrocarbonate (DEPC) at room temperature overnight and then autoclaved). The RNA concentration is determined by photometry.

The relative amount of ε-cyclase transcript in Tagetes leaves and flower stages is analyzed by RNA gel blotting as described in Sambrook & Russel (2001, Molecular Cloning: A laboratory manual, 3^{rd} Edition, Cold Spring Harbor Laboratory Press, Cold Spring Harbor, N.Y., Chapter 7, Protocol 6): about 10 to 15 μg of total RNA of each sample are fractionated in a formaldehyde agarose gel. The relative amounts of total RNA can be estimated from the rRNA bands stained with ethidium bromide (FIG. 1A). The amounts of ε-cyclase transcript are estimated by transferring the fractionated RNA by capillary blotting to a nylon membrane.

A radiolabeled ε-cyclase-specific probe was prepared by amplifying the fragment of SEQ ID NO: 47 (gecyc1) by polymerase chain reaction (PCR) from Tagetes erecta genomic DNA using a sense-specific primer (PR16=5′-ggcacgaggcaaagcaaagg-3′, SEQ ID NO: 48) and an antisense-specific primer (PR22=5′-cgataagtgcgacattcaagc-3′, SEQ ID NO: 49).

Tagetes erecta genomic DNA is prepared by harvesting leaf material from Tagetes erecta, freezing it in liquid nitrogen and powdering in a mortar. 100 mg of the frozen, powdered plant tissue is then transferred into a reaction vessel, taken up in 0.75 ml of extraction buffer and incubated at 65° C. for 60 min. The extraction buffer is freshly prepared from 25 ml of buffer 1 (0.35 M sorbitol, 0.1 M tris base, 5 μM EDTA, pH 7.5), 25 ml of buffer 2 (0.2 M tris base, 0.05 M EDTA, 2 M NaCL, 2% CTAB), 10 ml of 5% N-lauroylsarcosine sodium) and 0.24 g of sodium bisulfite. Incubation at 65° C. is followed by mixing the suspension with 0.7 ml of chloroform/isoamyl alcohol (24:1) and then centrifuging at 10 000 g for 5 min. The upper aqueous phase is transferred into a new reaction vessel, and the chloroform/isoamyl alcohol extraction is repeated as described. The upper aqueous phase is then transferred into a new reaction vessel, and the DNA is pelleted by adding 1 ml of isopropanol and then centrifuging at 10 000 g for 5 min. The DNA pellet is washed with 0.5 ml of 75% ethanol, then dried and subsequently resuspended in 0.05 ml of sterile water by incubation at 65° C. for 5 minutes.

The PCR conditions for amplification of an ε-cyclase-specific fragment from Tagetes erecta genomic DNA are as follows:

The PCR for amplifying an ε-cyclase-specific fragment takes place in a 50 μl reaction mixture which contains:
1 μg Tagetes erecta genomic DNA
0.25 μM dNTPs
0.2 μM primer PR16 (SEQ ID NO: 48)
0.2 μM primer PR22 (SEQ ID NO: 49)
5 μl 10× PCR buffer (TAKARA)
0.25 μl R Taq polymerase (TAKARA)
25.8 μl sterile distilled water

The PCR was carried out under the following cycle conditions: 1 cycle at 94° C. for 2 minutes. 35 cycles at 94° C. for 1 minute, 51° C. for 2 minutes and 72° C. for 3 minutes. Finally one cycle at 72° C. for 10 minutes.

The PCR amplification with PR16 and PR22 results in a 510 bp fragment (SEQ ID NO: 47) which, under stringent hybridization conditions, hybridizes specifically with the ε-cyclase but not with the lycopene β-cyclase from Tagetes erecta. The amplification product is purified using the NucleoSpin® extract kit (Machery & Nagel) as stated by the manufacturer and employed for a radiolabeling reaction with the Highprime® kit (Boehringer Mannheim) as stated by the manufacturer. The prehybridization, hybridization and washing steps are carried out as described in Sambrook & Russel (2001, Molecular Cloning: A laboratory manual, 3^{rd} Edition, Cold Spring Harbor Laboratory Press, Cold Spring Harbor, N.Y., Chapter 6, Protocol 10). The last washing step with 0.1×SSC/0.1% SDS at 65° C. makes the hybridization highly stringent, sufficient for specific detection with the probe described of ε-cyclase but not lycopene β-cyclase. The relative ε-cyclase transcript levels can be estimated from the hybridization signals detected with the aid of a phosphoimager. As is evident in FIG. 1B, under the given experimental conditions, the ε-cyclase transcript levels in the leaves are below the limit of detection, whereas large amounts of ε-cyclase transcripts are detectable throughout flower development.

### Example 2

### Cloning of the ε-cyclase Promoter

A 199 bp fragment or the 312 bp fragment of the Tagetes erecta ε-cyclase promoter can be isolated by two independent cloning strategies, inverse PCR (iPCR; adapted from Long et al. Proc Natl Acad Sci USA 90: 10370) and TAIL PCR (Liu Y G et al. (1995) Plant J 8: 457-463) using genomic DNA (as described above) from the Tagetes erecta line Orangenprinz.

For the iPCR mixture, 2 μg of genomic DNA are digested in a 25 μl reaction mixture with EcoRV and RsaI, then diluted to 300 μl and religated with 3 U of ligase at 16° C. overnight. PCR amplification using the primers PR50 (SEQ ID NO: 52) and PR51 (SEQ ID NO: 53) produces a fragment which comprises, in each case in sense orientation, 354 bp of the ε-cyclase cDNA (Genbank Acc. No.: AF251016) ligated to 312 bp of the ε-cyclase promoter and 70 bp of the 5′-terminal region of the ε-cyclase cDNA (see FIG. 7).

The conditions for the PCR reactions are as follows:

The PCR for amplifying the PR50-PR51 DNA fragment which comprises inter alia the 312 bp ε-cyclase promoter fragment takes place in a 50 μl reaction mixture containing:
1 μl ligation mixture (prepared as described above)
0.25 μM dNTPs
0.2 μM primer PR50 (SEQ ID NO: 52)
0.2 μM primer PR51 (SEQ ID NO: 53)
5 μl 10× PCR buffer (TAKARA)
0.25 μl R Taq polymerase (TAKARA)
28.8 μl sterile distilled water

The PCR reactions are carried out under the following cycle conditions: 1 cycle at 94° C. for 2 minutes. 35 cycles at 94° C. for 1 minute, 53° C. for 1 minute and 72° C. for 1 minute. Finally 1 cycle at 72° C. for 10 minutes.

The PCR amplification with primer PR50 and PR51 results in a 734 bp fragment which comprises inter alia the 312 bp ε-cyclase promoter fragment (FIG. 7). The amplicon is cloned using standard methods into the PCR cloning vector pCR2.1 (Invitrogen). Sequencings using the primers M13 and T7 result in the sequence SEQ ID NO: 50 for the amplicon.

For the TAIL PCR approach, three successive PCR reactions are carried out each with different gene-specific primers (“nested primers”).

The TAIL PCR takes place in a 20 μl reaction mixture containing:
100 ng genomic DNA (prepared as described above)
0.2 μM each dNTP
0.2 μM primer PR60 (SEQ ID NO: 54)
0.2 μM AD1 primer mixture
2 μl 10× PCR buffer (TAKARA)
0.5 U R Taq polymerase (TAKARA)
made up to 20 μl with sterile distilled water

The AD1 primer mixture initially represented a mixture of primers of the sequences

5′-(a/c/g/t)tcga(g/c)t(a/t)t(g/c)g(a/t)gtt-3′. The primer with the SEQ ID NO: 57 was found in the resulting amplicon.

The TAIL1 PCR reaction was carried out under the following cycle conditions:
1 cycle at 93° C. for 1 minute and 95° C. for 1 minute,
5 cycles at 94° C. for 30 seconds, 62° C. for 1 minute and 72° C. for 2.5 minutes,
1 cycle at 94° C. for 30 seconds, 25° C. for 3 minutes, then a temperature increase to 72° C. over the course of 3 minutes, 72° C. for 2.5 minutes
15 cycles at 94° C. for 10 seconds, 68° C. for 1 minute and 72° C. for 2.5 minutes; 94° C. for 10 seconds, 68° C. for 1 minute and 72° C. for 2.5 minutes; 94° C. for 10 seconds, 29° C. for 1 minute and 72° C. for 2.5 minutes;
1 cycle at 72° for 5 minutes.

The TAIL2 PCR takes place in a 21 μl reaction mixture containing;
1 μl of a 1:50 dilution of the TAIL1 reaction mixture (prepared as described above)
0.8 μM dNTP
0.2 μM primer PR61 (SEQ ID NO: 55)
0.2 μM primer AD1 (SEQ ID NO: 57)
2 μl 10× PCR buffer (TAKARA)
0.5 U R Taq polymerase (TAKARA)
made up to 21 μl with sterile distilled water

The TAIL2 PCR reaction is carried out under the following cycle conditions:
12 cycles at 94° C. for 10 seconds, 64° C. for 1 minute, 72° C. for 2.5 minutes, 94° C. for 10 seconds, 64° C. for 1 minute, 72° C. for 2.5 minutes; 94° C. for 10 seconds, 29° C. for 1 minute, 72° C. for 2.5 minutes;
1 cycle at 72° C. for 5 minutes.

The TAIL3 PCR takes place a 100 μl reaction mixture containing:
1 μl of a 1:10 dilution of the TAIL2 reaction mixture (prepared as described above)
0.8 μM dNTP
0.2 μM primer PR63 (SEQ ID NO: 56)
0.2 μM primer AD1 (SEQ ID NO: 57)
10 μl 10× PCR buffer (TAKARA)
0.5 U R Taq polymerase (TAKARA)
made up to 100 μl with sterile distilled water

The TAIL3 PCR reaction is carried out under the following cycle conditions:
20 cycles at 94° C. for 15 seconds, 29° C. for 30 seconds, 72° C. for 2 minutes;
1 cycle at 72° C. for 5 minutes.

The PCR amplification with primer PR63 and AD1 results in a 280 bp fragment which comprises inter alia the 199 bp ε-cyclase promoter fragment (FIG. 8).

The amplicon was cloned using standard methods into the PCR cloning vector pCR2.1 (Invitrogen). Sequencings using the primers M13 and T7 result in the sequence SEQ ID NO: 51. This sequence is identical in the overlap region to the sequence of SEQ ID NO: 50 which is isolated using the iPCR strategy, and thus represents the nucleotide sequence in the Tagetes erecta line Orangenprinz used.

The pCR2.1 clone which contains the 734 bp fragment (SEQ ID NO: 58) which is isolated by the iPCR strategy is called pTA-ecycP and is used to produce the expression constructs.

### Example 3

### Production of Transgenic ε-cyclase Expression Cassettes and Expression Vectors

The ε-cyclase regulatory element ecycP1 containing a promoter fragment and the 5′-untranslated region of the Tagetes erecta ε-cyclase is used to express β-glucuronidase (Jefferson et al. (1987) EMBO J 6:3901-3907) in tomato flowers (Lycopersicon esculentum). In addition, the ε-cyclase regulatory element ecycP2 containing a promoter fragment, the 5′-untranslated region and the putative transit peptide of the Tagetes erecta ε-cyclase is used to express either β-glucuronidase or the Haematococcus pluvialis ketolase in plastids of tomato flowers.

The transgenic expression vectors pEcycP1:GUS, pEcycP2:GUS, pEcycP2:KETO for the agrobacterium-mediated transformation into Lycopersicon esculentum were produced using the binary vector pS0301 (WO 02/00900). The transformation plasmids are produced by producing the fragments ecycP1 and ecycP2 by PCR using the clone pTA-ecycP and the primers OL1 (SEQ ID NO: 59) and OL2 (SEQ ID NO: 60) (for ecycP1) or the primers OL1 (SEQ ID NO: 59) and OL3 (SEQ ID NO: 61) (for ecycP2).

The PCR for amplifying an ε-cyclase-specific fragment takes place in a 50 μl reaction mixture containing:
50 ng pTA-ecycP plasmid
0.25 μM dNTPs
0.2 μM primer OL1 (SEQ ID NO: 59)
0.2 μM primer OL2 (SEQ ID NO: 60) for ecycP1 or primer OL3 (SEQ ID NO: 61) for ecycP2
5 μl 10× PCR buffer (TAKARA)
0.25 μl R Taq polymerase (TAKARA)
25.8 μl sterile distilled water.

The PCR is carried out under the following cycle conditions: 1 cycle at 94° C. for 2 minutes, 35 cycles at 94° C. for 1 minute, 50° C. for 2 minutes and 72° C. for 3 minutes, finally 1 cycle at 72° C. for 10 minutes.

The PCR amplification with OL1 and OL2 results in a 456 bp fragment (ecycP1, SEQ ID NO: 5), the PCR amplification with OL1 and OL3 results in a 543 bp fragment (ecycP2, SEQ ID NO:_{—}6). The amplicons ecycP1 and ecycP2 are cloned using standard methods into the PCR cloning vector pCR2.1 (Invitrogen), and the clones pTA-ecycP1 and pTA-ecycP2 are obtained. Sequencings of the two clones confirms sequences which are identical in their respective overlap region to SEQ ID NO: 47 and SEQ ID NO: 58, respectively. These clones are therefore used for ligation into the transformation vector pS0301 (WO 02/00900).

The transformation plasmid pEcycP1:GUS is produced by isolating the 454 bp XhoI-NcoI ecycP1 fragment from pTA-ecycP1 and ligating into the XhoI-NcoI-cut vector pS0301. The clone containing the ecycP1 fragment in the correct orientation is called pEcycP1:GUS (FIG. 2, construct map).

The transformation plasmid pEcycP2:GUS is produced by isolating the 541 bp XhoI-NcoI ecycP2 fragment from pTA-ecycP2 and ligating into the XhoI-NcoI-cut vector pS0301. The clone containing the ecycP2 fragment in the correct orientation is called pEcycP2:GUS (FIG. 3, construct map).

The transformation plasmid pEcycP2:KETO is produced by replacing the region “GUSI/intron/GUSII/35ST” bonded by an NcoI and a HindIII restriction cleavage site in pEcycP2:GUS by a “ketolase/35S terminator” region. For this purpose, the plasmid pEcycP2:GUS is linearized by standard methods with HindIII, and the resulting 5′ overhangs are filled in with Klenow fragment and finally the “GUSI/intron/GUSII/35ST” region is deleted by restriction digestion with NcoI.

The “ketolase/35S terminator” region is produced by
1. cloning a ketolase cDNA produced using RNA isolated from Haematococcus pluvialis (Flotow em. Wille), followed by
2. producing a transcriptional ketolase/terminator fusion by ligating the ketolase sequence into the vector pJIT117, which then serves as template for
3. the PCR amplification of the ketolase/35S terminator region.

The cDNA which codes for the Haematococcus pluvialis ketolase is amplified by PCR from Haematococcus pluvialis (strain 192.80 of the “Sammlung von Algenkulturen der Universität Göttingen”) suspension culture.

To prepare total RNA from a suspension culture of Haematococcus pluvialis (strain 192.80) which is grown for two weeks in indirect daylight at room temperature in Haematococcus medium (1.2 g/l sodium acetate, 2 g/l yeast extract, 0.2 g/l MgCl₂×6 H₂O, 0.02 CaCl₂×2 H₂O; pH 6.8; addition of 400 mg/l L-asparagine, 10 mg/l FeSO₄×H₂O after autoclaving), the cells are harvested, frozen in liquid nitrogen and powdered in a mortar. 100 mg of the frozen, powdered algal cells are then transferred into a reaction vessel and taken up in 0.8 ml of Trizol® buffer (LifeTechnologies). The suspension is extracted with 0.2 ml of chloroform. After centrifugation at 12 000 g for 15 minutes, the aqueous supernatant is removed and transferred into a new reaction vessel and extracted with one volume of ethanol. The RNA is precipitated with one volume of isopropanol and washed with 75% ethanol, and the pellet is dissolved in DEPC water (water incubated with 1/1000 volume of diethyl pyrocarbonate at room temperature overnight and then autoclaved). The RNA concentration is determined by photometry.

For the cDNA synthesis, 2.5 μg of total RNA are denatured at 60° C. for 10 min, cooled on ice for 2 min and transcribed into cDNA using a cDNA kit (Ready-to-go-you-prime-beads®, Pharmacia Biotech) as stated by the manufacturer using an antisense-specific primer (PR1 SEQ ID NO: 64).

The nucleic acid encoding a ketolase from Haematococcus pluvialis (strain 192.80) is amplified by the polymer chain reaction (PCR) from Haematococcus pluvialis cDNA using a sense-specific primer (PR2; SEQ ID NO: 65) and an antisense-specific primer (PR1; SEQ ID NO: 64). The PCR conditions are as follows:

The PCR for amplifying the cDNA which codes for a ketolase protein consisting of the complete primary sequence takes place in a 50 μl reaction mixture containing:
4 μl a Haematococcus pluvialis cDNA (prepared as described above)
0.25 μM dNTPs
0.2 μM primer PR1 (SEQ ID NO: 64)
0.2 μM primer PR2 (SEQ ID NO: 65)
5 μl 10× PCR buffer (TAKARA)
0.25 μl R Taq polymerase (TAKARA)
25.8 μl sterile distilled water

The PCR is carried out under the following cycle conditions: 1 cycle at 94° C. for 2 minutes; 35 cycles at 94° C. for 1 minute, 53° C. for 2 minutes and 72° C. for 3 minutes. Finally 1 cycle at 72° C. for 10 minutes.

The PCR amplification with PR1 and PR2 results in an 1155 bp fragment which codes for a protein consisting of the complete primary sequence. Standard methods are used to clone the ketolase amplicon into the PCR cloning vector pGEM-Teasy (Promega), and the clone pGKETO2 is obtained.

Sequencing of the clone pGKETO2 with the T7 primer and the SP6 primer confirms a sequence which differs only in the three codons 73, 114 and 119, in one base in each case, from the published sequence (Genbank Acc. No.: X86782). These nucleotide exchanges are produced in an independent amplification experiment and thus represent the nucleotide sequence in the Haematococcus pluvialis strain 192.80 used (FIGS. 9 and 10, sequence comparisons). This clone is used for cloning into the expression vector pJIT117 (Guerineau et al. (1988) Nucl Acids Res 16: 11380). Further cloning takes place by isolating the 1031 bp SpHI fragment from pGKETO2 and ligating into the SpHI cut vector pJIT117. The clone containing the Haematococcus pluvialis ketolase in the correct orientation as N-terminal translational fusion with the rbcs transitpeptide is called pJKETO2.

The 1795 bp ketolase/35S terminator region is produced by PCR using pJKETO2 and the primers OL4 (SEQ ID NO: 62) and OL5 (SEQ ID NO: 63). The conditions of the PCR reactions are as follows:

The PCR for amplifying the OL4-OL5 DNA fragment which contains the coding region of the ketolase followed by the 35S terminator from CaMV takes place in a 50 μl reaction mixture containing:
1 μl pJKETO2 (1 ng of plasmid DNA)
0.25 μM dNTPs
0.2 μM primer OL4 (SEQ ID NO: 62)
0.2 μM primer OL5 (SEQ ID NO: 63)
5 μl 10× PCR buffer (TAKARA)
0.25 μl R Taq polymerase (TAKARA)
28.8 μl sterile distilled water

The PCR reactions are carried out under the following cycle conditions: 1 cycle at 94° C. for 2 minutes. 35 cycles at 94° C. for 1 minute, 53° C. for 2 minutes and 72° C. for 3 minutes. Finally 1 cycle at 72° C. for 10 minutes.

The PCR amplification with primer OL4 and OL5 results in a 1795 bp fragment which contains the coding region of the ketolase followed by the 35S terminator from CaMV. This 1795 bp amplicon is cloned by using standard methods into the PCR cloning vector pCR2.1 (Invitrogen), and the clone “pTA-KETO/Term” is obtained. Sequencing of the clone confirms a sequence which is identical in the respective overlap region to SEQ ID NO: 66 and pJIT117. This clone is therefore used for the ligation into the transformation vector pEcycP2:GUS (see above). The transformation plasmid pEcycP2:KETO is produced by isolating the 1791 bp NcoI-EcoRV “KETO/Term” fragment from pTA-KETO/Term and ligating it into the linearized vector pEcycP2:GUS containing an NcoI-5′ overhang and a blunt end. The clone containing the ecycP2 fragment in the correct orientation is called pEcycP2:KETO (FIG. 4, construct map).

### Example 4

### Production and Analysis of Transgenic Tomato Plants

The constructs pEcycP1:GUS, pEcycP2:GUS and pEcycP2:KETO were transformed by Agrobacterium tumefaciens-mediated transformation into tomato. Cotyledons and hypocotyls of seedlings seven to ten days old of the Microtom line are used as initial explant for the transformation. The culture medium of Murashige and Skoog (Murashige & Skoog (1962) Physiol Plant 15, 473-497) with 2% sucrose, pH 6.1, is used for germination. Germination takes place at 21° C. with low light (20 to 100 μE). After seven to ten days, the cotyledons are divided transversely, and the hypocotyls are cut into sections about 5 to 10 mm long and placed on the MSBN medium (MS, pH 6.1, 3% sucrose with 1 mg/l benzylaminopurine (BAP), 0.1 mg/l naphthaleneacetate (NAA)) which have been charged the previous day with suspension-cultivated tomato cells. The tomato cells are covered, free of air bubbles, with sterile filter paper. The explants are precultured on the described medium for three to five days. The explants are then infected with the Agrobacterium tumefaciens strain LBA4404, which harbors the binary plasmid with the gene to be transformed, as follows: the strain, which has been cultivated in YEB medium with the antibiotic for the binary plasmid at 28° C. overnight, is centrifuged. The bacterial pellet is resuspended in liquid MS medium (3% sucrose, pH 6.1) and adjusted to an optical density of 0.3 (at 600 nm). The precultured explants are transferred into the suspension and incubated at room temperature, shaking gently, for 30 minutes. The explants are then dried with sterile filter paper and returned to their preculture medium for the three-day coculture (21° C.).

After the coculture, the explants are transferred to MSZ2 medium (MS pH 6.1 with 3% sucrose, 2 mg/l zeatin, 100 mg/l kanamycin, 160 mg/l timentin) and stored for the selective regeneration at 21° C. under weak light conditions (20 to 100 μE, light/dark rhythm 16h/8h). The explants are transferred every two to three weeks until shoots form. Small shoots can be detached from the explant and rooted on MS (pH 6.1 with 3% sucrose), 160 mg/l timentin, 30 mg/l kanamycin, 0.1 mg/l IAA. Rooted plants are transferred into the glasshouse.

The transgenicity of rooted tomato plants is confirmed by PCR using genomic DNA. The activity profile of the ε-cyclase promoter fragment can be investigated in the case of the ecycP:GUS construct by a GUS assay by standard methods (Jefferson et al. (1987) EMBO J 6:3901-3907). The activity profile of the ε-cyclase promoter fragment can be investigated in the case of the pEcycP2:KETO construct by Northern blot analysis by standard methods using a ketolase-specific hybridization probe or by ketolase-specific real-time PCR (Sambrook & Russel, 2001, Molecular Cloning: A laboratory manual, 3^{rd} Edition, Cold Spring Harbor Laboratory Press, Cold Spring Harbor, N.Y.).

### Example 5

### Production of a Transgenic Expression Vector for Producing Double-Stranded ε-cyclase Ribonucleic Acid Sequences

Expression of inverted repeat transcripts consisting of fragments of the ε-cyclase promoter in Tagetes erecta takes place under the control of a modified version (AP3P) of the flower-specific promoter AP3 from Arabidopsis thaliana (GenBank Acc. No.: AL132971: Nucleotide region 9298 to 10200; Hill et al. (1998) Development 125:1711-1721). The inverted repeat transcript comprises in each case a fragment in the correct orientation (sense fragment) and a sequence-identical fragment in the contrary orientation (antisense fragment), which are connected together by a functional intron, the PIV2 intron of the potato ST-LH1 gene (Vancanneyt G et al. (1990) Mol Gen Genet 220:245-50).

The cDNA which codes for the AP3 promoter (−902 to +15) from Arabidopsis thaliana is produced by PCR using genomic DNA (isolated from Arabidopsis thaliana by a standard method) and the primers PR7 (SEQ ID NO: 67) and PR10 (SEQ ID NO: 70). The PCR conditions are as follows:

The PCR for amplifying the DNA which encodes the AP3 promoter fragment (−902 to +15) takes in place in a 50 μl reaction mixture containing:
1 μl (equivalent to 20 ng) of genomic DNA from A. thaliana (1:100 dil.; prepared as described above)
0.25 mM dNTPs
0.2 μM primer PR7 (SEQ ID NO: 67)
0.2 μM primer PR10 (SEQ ID NO: 70)
5 μl 10× PCR buffer (Stratagene)
0.25 μl Pfu polymerase (Stratagene)
28.8 μl sterile distilled water.

The PCR is carried out under the following cycle conditions: 1 cycle at 94° C. for 2 minutes. 35 cycles at 94° C. for 1 minute, 50° C. for 1 minute and 72° C. for 1 minute. Finally 1 cycle at 72° C. for 10 minutes.

The 922 bp amplicon is cloned using standard methods into the PCR cloning vector pCR 2.1 (Invitrogen), and the plasmid pTAP3 is obtained. Sequencing of the clone pTAP3 confirms a sequence which differs merely by an insertion (a G in position 9765 of the GenBank Acc. No.: AL132971 sequence) and a base exchange (a G in place of an A in position 9726 of the GenBank Acc. No.: AL132971 sequence) from the published AP3 sequence (GenBank Acc. No.: AL132971, nucleotide region 9298 to 10200) (position 33: T instead of G, position 55: T instead of G). These nucleotide differences can be reproduced in an independent amplification experiment and thus represent the nucleotide sequence in the Arabidopsis thaliana plant used.

The modified version AP3P is produced by recombinant PCR using the plasmid pTAP3. The region 10200 to 9771 is amplified using the primers PR7 (SEQ ID NO: 67) and PR9 (SEQ ID NO: 69) (amplicon A7/9), and the region 9526 to 9285 was amplified with the primers PR8 (SEQ ID NO: 68) and PR10 (SEQ ID NO: 70) (amplicon A8/10). The PCR conditions are as follows:

The PCR reaction for amplifying the DNA fragments which code for the regions 10200 to 9771 and 9526 to 9285 of the AP3 promoter takes place in 50 μl reaction mixtures containing:
100 ng AP3 amplicon (described above)
0.25 mM dNTPs
0.2 μM primer PR7 (SEQ ID NO: 67) or primer PR8 (SEQ ID NO: 68)
0.2 μM primer PR9 (SEQ ID NO: 69) or primer PR10 (SEQ ID NO: 70)
5 μl 10× PCR buffer (Stratagene)
0.25 μl Pfu Taq polymerase (Stratagene)
28.8 μl sterile distilled water

The PCR is carried out under the following cycle conditions: 1 cycle at 94° C. for 2 minutes. 35 cycles at 94° C. for 1 minute, 50° C. for 2 minutes and 72° C. for 3 minutes. Finally 1 cycle at 72° C. for 10 minutes.

The recombinant PCR comprises annealing of the amplicons A7/9 and A8/10 which overlap over a sequence of 25 nucleotides, completion of a double strand and subsequence amplification. This results in a modified version of the AP3 promoter (AP3P) in which the positions 9670 to 9526 are deleted. Denaturation (5 min at 95° C.) and annealing (slow cooling at room temperature to 40° C.) of the two amplicons A7/9 and A8/10 takes place in a 17.6 μl reaction mixture containing:
0.5 μg A7/9
0.25 μg A8/10

Filling-in of the 3′ ends (30 min at 30° C.), takes place in a 20 μl reaction mixture containing:
17.6 μl A7/9 and A8/10 annealing reaction (prepared as described above)
50 μM dNTPs
2 μl 1× Klenow buffer
2 U Klenow enzyme

The nucleic acid coding for the modified promoter version AP3P is amplified by PCR using a sense-specific primer (PR7 SEQ ID NO: 67) and an antisense-specific primer (PR10 SEQ ID NO: 70). The PCR conditions are as follows:

The PCR for amplifying the AP3P fragment takes place in a 50 μl reaction mixture containing:
1 μl annealing reaction (prepared as described above)
0.25 mM dNTPs
0.2 μM primer PR7 (SEQ ID NO: 67)
0.2 μM primer PR10 (SEQ ID NO: 70)
5 μl 10× PCR buffer (Stratagene)
0.25 μl Pfu Taq polymerase (Stratagene)
28.8 μl sterile distilled water

The PCR is carried out under the following cycle conditions: 1 cycle at 94° C. for 2 minutes. 35 cycles at 94° C. for 1 minute, 50° C. for 1 minute and 72° C. for 1 minute. Finally 1 cycle at 72° C. for 10 minutes.

The PCR amplification with the primers PR7 (SEQ ID NO: 67) and PR10 (SEQ ID NO: 70) results in a 777 bp fragment which codes for the modified promoter version AP3P (SEQ ID NO: 77). The amplicon is cloned into the cloning vector pCR2.1 (Invitrogen). Sequencings with the primers T7 and M13 confirm a sequence identical to the sequence of GenBank Acc. No.: AL132971, region 10200 to 9298, the internal region from 9285 to 9526 being deleted. This clone is used for cloning into the expression vector pJIT117 (Guerineau et al. (1988) Nucl Acids Res 16:11380).

The cloning takes place by isolating the 775 bp SacI-HindIII fragment from pTAP3P and ligating into the SacI-HindIII-cut vector pJIT117. The clone which contains the promoter AP3P in place of the original promoter d35S is called pJAP3P.

A DNA fragment which comprises the PIV2 intron of the ST-LS1 gene is produced by PCR using p35SGUS INT plasmid DNA (Vancanneyt G. et al.(1990) Mol Gen Genet 220:245-250) and the primers PR40 (SEQ ID NO: 71) and PR41 (SEQ ID NO: 72). The PCR conditions are as follows:

The PCR for amplifying the sequence of the PIV2 intron of the ST-LS1 gene takes place in a 50 μl reaction mixture containing:
50 ng p35SGUS INT
0.25 mM dNTPs
0.2 μM primer PR40 (SEQ ID NO: 71)
0.2 μM primer PR41 (SEQ ID NO: 72)
5 μl 10× PCR buffer (TAKARA)
0.25 μl R Taq polymerase (TAKARA)
28.8 μl sterile distilled water

The PCR is carried out under the following cycle conditions: 1 cycle at 94° C. for 2 minutes. 35 cycles at 94° C. for 1 minute, 53° C. for 1 minute and 72° C. for 1 minute. Finally 1 cycle at 72° C. for 10 minutes.

The PCR amplification with PR40 and PR41 results in a 212 bp fragment (SEQ ID NO: 78). The amplicon is cloned using standard methods into the PCR cloning vector pBluntII (Invitrogen), and the clone pBluntII-40-41 is obtained. Sequencing of this clone with the primer SP6 confirms a sequence which is identical to the corresponding sequence from the vector p35SGUS INT. This clone is employed for cloning into the vector pJAP3P (see above). The cloning takes place by isolating the 210 bp SalI-BamHI fragment from pBluntII-40-41 and ligating with the SalI-BamHI-cut vector pJAP3P. The clone which contains the PIV2 intron of the ST-LS1 gene in the correct orientation following the 3′ end of the rbcs transit peptide is called pJAI1 and is suitable for producing expression cassettes for the flower-specific expression of inverted repeat transcripts.

### Example 6

### Production of Inverted Repeat Expression Cassettes for Flower-Specific Expression of ε-cyclase Promoter dsRNAs in Tagetes erecta

Expression of inverted repeat transcripts consisting of ε-cyclase promoter fragments in Tagetes erecta took place under the control of a modified version (AP3P) of the flower-specific promoter AP3 from Arabidopsis (see example 5) or of the flower-specific promoter CHRC (Genbank Acc. No. AF099501). The inverted repeat transcript contains in each case an ε-cyclase promoter fragment in the correct orientation (sense fragment) and a sequence-identical ε-cyclase promoter fragment in the contrary orientation (antisense fragment), which are connected together by a functional intron (see example 5).

The promoter fragments are produced by PCR using plasmid DNA (clone pTA-ecycP, see example 2) and the primers PR124 (SEQ ID NO: 73) and PR126 (SEQ ID NO: 75) or the primers PR125 (SEQ ID NO: 74) and PR127 (SEQ ID NO: 76). The conditions of the PCR reactions are as follows:

The PCR for amplifying the PR124-PR126 DNA fragment which contains the ε-cyclase promoter fragment takes place in a 50 μl reaction mixture containing:
1 μl pTA-ecycP (10 ng/μl; see example 2)
0.25 mM dNTPs
0.2 μM primer PR124 (SEQ ID NO: 73)
0.2 μM primer PR126 (SEQ ID NO: 75)
5 μl 10× PCR buffer (Stratagene)
0.25 μl Pfu polymerase (Stratagene)
28.8 μl sterile distilled water

The PCR for amplifying the PR125-PR127 DNA fragment containing the 312 bp ε-cyclase promoter fragment takes place in a 50 μl reaction mixture containing:
1 μl pTA-ecycP (10 ng/μl; see example 2)
0.25 mM dNTPs
0.2 μM primer PR125 (SEQ ID NO: 74)
0.2 μM primer PR127 (SEQ ID NO: 76)
5 μl 10× PCR buffer (Stratagene)
0.25 μl Pfu polymerase (Stratagene)
28.8 μl sterile distilled water

The PCR reactions are carried out under the following cycle conditions: 1 cycle at 94° C. for 2 minutes. 35 cycles at 94° C. for 1 minute, 53° C. for 1 minute and 72° C. for 1 minute. Finally 1 cycle at 72° C. for 10 minutes.

The PCR amplification with primer PR124 and PR126 results in a 358 bp fragment, and PCR amplification with primer PR125 and PR127 resulted in a 361 bp fragment.

The two amplicons, the PR124-PR126 (HindIII-SalI sense) fragment and the PR125-PR127 (EcoRI-BamHI antisense) fragment, are cloned using standard methods into the PCR cloning vector pCR-BluntII (Invitrogen). Sequencings with the primer SP6 confirm in each case a sequence which, apart from the introduced restriction sites, is identical to SEQ ID NO: 58. These clones are therefore used to produce an inverted repeat construct in the cloning vector pJAI1 (see example 5).

The first cloning step takes place by isolating the 356 bp PR124-PR126 HindIII-SalI fragment from the cloning vector pCR-BluntII (Invitrogen) and ligating with the HindIII-SalI-cut vector pJAI1. The clone containing the ε-cyclase promoter fragment in the sense orientation is called cs43. The ligation results in the sense fragment of the ε-cyclase promoter being inserted between the AP3P promoter and the intron. The second cloning step takes place by isolating the 359 bp PR125-PR127 BamHI-EcoRI fragment from the cloning vector pCR-BluntII (Invitrogen) and ligating with BamHI-EcoRI-cut vector cs43. The clone containing the ε-cyclase promoter fragment in the antisense orientation is called cs44. The ligation results in a transcriptional fusion between the intron and the antisense fragment of the ε-cyclase promoter.

An inverted repeat expression cassette under the control of the CHRC promoter is produced by amplifying a CHRC promoter fragment using genomic petunia DNA (prepared by standard methods) and the primers PRCHRC5′ (SEQ ID NO 82) and PRCHRC3′ (SEQ ID NO: 83). The amplicon is cloned into the cloning vector pCR2.1 (Invitrogen). Sequencings of the resulting clone pCR2.1-CHRC with the primers M13 and T7 confirm a sequence identical to the GenBank Acc. No.: AF099501 sequence. This clone is therefore used for cloning into the expression vector cs44. The cloning takes place by isolating the 1535 bp SacI-HindIII fragment from pCR2.1-CHRC and ligating into the SacI-HindIII-cut vector cs44. The clone which contains the CHRC promoter in place of the original AP3P promoter is called cs45.

The transformation plasmids for the agrobacterium-mediated transformation of the AP3P-controlled inverted repeat transcript in Tagetes erecta are produced using the binary vector pSUN5 (WO 02/00900).

The transformation plasmid pS5AI7 is produced by ligating the 1683 bp SacI-XhoI fragment from cs44 with the SacI-XhoI-cut vector pSUN5 (FIG. 5, construct map).

The transformation plasmid pS5CI7 is produced by ligating the 2448 bp SacI-XhoI fragment from cs45 with the SacI-XhoI-cut vector pSUN5 (FIG. 6, construct map).

### Example 7

### Production and Analysis of Transgenic Tagetes Plants

The transformation plasmids pS5AI7 and pS5CI7 are transformed by Agrobacterium tumefaciens-mediated transformation into Tagetes.

Tagetes seeds are sterilized and placed on germination medium (MS medium; Murashige & Skoog (1962) Physiol Plant 15:473-497; pH 5.8, 2% sucrose). Germination takes place in a temperture/light/time interval of 18 to 28° C./20 to 200 μE/3 to 16 weeks, but preferably at 21° C., 20 to 70 μE, for 4 to 8 weeks.

All leaves of the plants which have developed in vitro by then are harvested and cut transverse to the middle. The leaf explants resulting therefrom, with a size of 10 to 60 mm², are stored during the preparation in liquid MS medium at room temperature for not more than 2 h.

Any Agrobacterium tumefaciens strain, but preferably a supervirulent strain such as, for example, EHA105 with an appropriate binary plasmid, which may harbor a selection marker gene (preferably bar or pat) and one or more trait or reporter genes is cultivated overnight and used for cocultivation with the leaf material. The bacterial strain can be cultured as follows: a single colony of the appropriate strain is inoculated in YEB (0.1% yeast extract, 0.5% beef extract, 0.5% peptone, 0.5% sucrose, 0.5% magnesium sulfate×7 H₂O) with 25 mg/l kanamycin and cultured at 28° C. for 16 to 20 h. The bacterial suspension is then harvested by centrifugation at 6000 g for 10 min, and resuspended in liquid MS medium so as to result in an OD₆₀₀ of about 0.1 to 0.8.

Immediately before the cocultivation, the MS medium in which the leaves have been stored is replaced by the bacterial suspension. Incubation of the leaves in the agrobacteria suspension took place at room temperature with gentle shaking for 30 min. The infected explants are then put on an MS medium solified with agar (e.g. 0.8% plant agar (Duchefa, N L)), with growth regulators such as, for example, 3 mg/l benzylaminopurine (BAP) and 1 mg/l indolylacetic acid (IAA). The orientation of the leaves on the medium is immaterial. Cultivation of the explants takes place for 1 to 8 days, but preferably for 6 days, during which the following conditions can be used: light intensity: 30 to 80 μmol/m²× sec, temperature: 22 to 24° C., 16/18 hours light/dark alternation. The cocultivated explants are then transferred to fresh MS medium, preferably with the same growth regulators, this second medium additionally containing an antibiotic to suppress bacterial growth. Timentin in a concentration of 200 to 500 mg/l is very suitable for this purpose. The second selective component employed is one for selecting for successful transformation. Phosphinothricin in a concentration of 1 to 5 mg/l selects very efficiently, but other selective components according to the method to be used are also conceivable. After one to three weeks in each case, the explants are transferred to fresh medium until plumules and small shoots develop, which are then transferred to the same basal medium including timentin and PPT or alternative components with growth regulators, namely, for example, 0.5 mg/l indolylbutyric acid (IBA) and 0.5 mg/l gibberillic acid GA₃, for rooting. Rooted shoots can be transferred into the glasshouse.

In addition to the method described, the following advantageous modifications are possible:
before the explants are infected with the bacteria, they can be preincubated on the medium described above for the cocultivation for 1 to 12 days, preferably 3 to 4. This is followed by infection, cocultivation and selection regeneration as described above.
the pH for the regeneration (normally 5.8) can be lowered to pH 5.2. This improves control of the growth of agrobacteria.
addition of AgNO₃ (3 to 10 mg/l) to the regeneration medium improves the condition of the culture, including the regeneration itself.
components which reduce phenol formation and are known to the skilled worker, such as, for example citric acid, ascorbic acid, PVP and many others, have beneficial effects on the culture.
liquid culture medium can also be used for the whole method. The culture can also be incubated on commercially available supports which are positioned on the liquid medium.

The transgenicity of rooted shoots can be investigated on isolated genomic DNA by the polymerase chain reaction (PCR). The reduction in the amounts of ε-cyclase transcript (compared with the wild type used for the transformation) as a result of transformation with the transformation plasmid pS5AI7 or pS5CI7 can be investigated by Northern gel blot analysis by standard methods (Sambrook & Russel, 2001, Molecular Cloning: A laboratory manual, 3^{rd} Edition, Cold Spring Harbor Laboratory Press, Cold Spring Harbor, N.Y.) using an ε-cyclase-specific hybridization probe, for example produced as described in example 1. In addition, the reduction in the amounts of ε-cyclase transcript (compared with the wild type used for the transformation) can be investigated by ε-cyclase-specific real time PCR.

1. A method for the transgenic expression of nucleic acid sequences in the flower of plants, including the following steps
I. introduction of a transgenic expression cassette into plant cells, where the transgenic expression cassette comprises at least the following elements
a) at least one promoter sequence of a gene coding for an ε-cyclase, and
b) at least one further nucleic acid sequence, and
c) where appropriate further genetic control elements,
where at least one of said promoter sequences and one further nucleic acid sequence are functionally linked together, and the further nucleic acid sequence is heterologous in relation to the promoter sequence or the plant cell, and
II. selection of transgenic cells which comprise said expression cassette stably integrated into the genome, and
III. regeneration of complete plants from said transgenic cells, where at least one of the further nucleic acid sequences is expressed in the flower.

2. The method as claimed in claim 1, where the promoter sequence of a gene coding for an ε-cyclase is a sequence selected from the group of sequences consisting of p1 i) the promoter sequence of the ε-cyclase from Tagetes erecta as shown in SEQ ID NO: 1, the ε-cyclase from Arabidopsis thaliana as shown in SEQ ID NO: 7, the ε-cyclase from Oryza sativa as shown in SEQ ID NO: 8, and
ii) functional equivalents of the promoter sequences as shown in SEQ ID NO: 1, 7 or 8 having substantially the same promoter activity as the promoter of the ε-cyclases as shown in SEQ ID NO: 1, 7 or 8 and
iii) functionally equivalent fragments of the sequences under i) or ii) having substantially the same promoter activity as the promoter of ε-cyclases as shown in SEQ ID NO: 1, 7 or 8.

3. A method for identifying and/or isolating promoters of genes which code for an ε-cyclase, where at least one nucleic acid sequence or a part thereof is employed in the identification and/or isolation, where said nucleic acid sequence codes for an amino acid sequence which comprises at least one sequence as shown in SEQ ID NO: 17, 18, 19, 20, 21 or 22 or a variation of these sequences.

4. The method as claimed in claim 3, where said nucleic acid sequence comprises a sequence as shown in SEQ ID NO: 23, 25, 27, 29, 31, 33, 35, 37, 39, 41, 43 or 45.

5. The method as claimed in claim 3, where the method is carried out with use of the polymerase chain reaction, and said nucleic acid sequence or a part thereof is employed as primer.

6. A method for producing a transgenic expression cassette with specificity for the flower of plants, including the following steps:
I. isolation of a promoter sequence, where at least one nucleic acid sequence or a part thereof is employed in the isolation, where said nucleic acid sequence codes for an amino acid sequence which comprises at least one sequence as shown in SEQ ID NO: 17, 18, 19, 20, 21 or 22 or a variation of these sequence motifs.
II. functional linkage of said promoter sequence to a further nucleic acid sequence, where said nucleic acid sequence is heterologous in relation to the promoter.

7. The method as claimed in claim 6, where said nucleic acid sequence includes a sequence as shown in SEQ ID NO: 23, 25, 27, 29, 29, 31, 33, 35, 37, 39, 41, 43 or 45.

8. The method as claimed in claim 6, where the method is carried out with use of the polymerase chain reaction, and said nucleic acid sequence or a part thereof is employed as primer.

9. A transgenic expression cassette for the targeted transgenic expression of nucleic acid sequences in the flower of plants, including
a) at least one promoter sequence of a gene coding for an ε-cyclase, and
b) at least one further nucleic acid sequence, and
c) where appropriate further genetic control elements,
where at least one promoter sequence and one further nucleic acid sequence are functionally linked together, and the further nucleic acid sequence is heterologous in relation to the promoter sequence.

10. The transgenic expression cassette as claimed in claim 9, where the promoter sequence of a gene coding for an ε-cyclase is a sequence selected from the group of sequences consisting of
i) the promoter sequence of the ε-cyclase from Tagetes erecta as shown in SEQ ID NO: 1, the ε-cyclase from Arabidopsis thaliana as shown in SEQ ID NO: 7, the ε-cyclase from Oryza sativa as shown in SEQ ID NO: 8, and
ii) functional equivalents of the promoter sequences as shown in SEQ ID NO: 1, 7 or 8 having substantially the same promoter activity as the promoter of the ε-cyclases as shown in SEQ ID NO: 1, 7 or 8 and
iii) functionally equivalent fragments of the sequences under i) or ii) having substantially the same promoter activity as the promoter of ε-cyclases as shown in SEQ ID NO: 1, 7 or 8.

11. The transgenic expression cassette as claimed in claim 9, where the nucleic acid sequence to be expressed transgenically enables
a) the expression of a protein encoded by said nucleic acid sequence, or
b) the expression of a sense RNA, antisense RNA or double-stranded RNA encoded by said nucleic acid sequence.

12. An isolated nucleic acid sequence comprising
a) the Tagetes erecta ε-cyclase promoter as shown in SEQ ID NO: 1 or
b) a functionally equivalent fragment of a) with substantially the same promoter activity as a).

13. The isolated nucleic acid sequence as claimed in claim 12, including, in the 3′ orientation to the Tagetes erecta ε-cyclase promoter as shown in SEQ ID NO: 1 or a functionally equivalent fragment of the aforementioned, a sequence coding for a 5′-untranslated region and/or a transit peptide.

14. The isolated nucleic acid sequence as claimed in claim 12 including a sequence described by SEQ ID NO: 2 or 3.

15. A double-stranded RNA molecule comprising
a) a sense RNA strand comprising at least one ribonucleotide sequence which is substantially identical to at least part of a nucleic acid sequence coding for the promoter region of an ε-cyclase, and
b) an antisense RNA strand which is substantially complementary to the RNA sense strand under a).

16. The double-stranded RNA molecule as claimed in claim 15, where the promoter region of the ε-cyclase comprises a sequence selected from the sequences as shown in SEQ ID NO: 1, 7 or 8.

17. A ribonucleic acid molecule comprising
a) at least one ribonucleotide sequence which is substantially identical to at least one part of a nucleic acid sequence coding for the promoter region of an ε-cyclase, and
b) at least one further ribonucleotide sequence which is substantially complementary to at least one part of the ribonucleotide sequence under a),
where a) and b) are connected together covalently, and further functional elements may be located where appropriate between a) and b).

18. The ribonucleic acid molecule as claimed in claim 17, where the promoter region of the ε-cyclase includes a sequence selected from the sequences as shown in SEQ ID NO: 1, 7 or 8.

19. A transgenic expression cassette, comprising
a) at least one promoter functional in plants, and
b) at least one nucleic acid sequence coding for a double-stranded RNA molecule comprising
i) a sense RNA strand comprising at least one ribonucleotide sequence which is substantially identical to at least part of a nucleic acid sequence coding for the promoter region of an ε-cyclase, and
ii) an antisense RNA strand which is substantially complementary to the RNA sense strand under i)
or coding for the ribonucleic acid molecule as claimed in claim 17
where at least one of said promoters and at least one of said nucleic acid sequences are functionally linked together, and the promoter is heterologous in relation to the nucleic acid sequence.

20. The transgenic expression cassette as claimed in claim 19, where the promoter is a promoter having specificity for the flower of plants.

21. A transgenic expression vector comprising the nucleic acid sequence as claimed in claim 12.

22. A transgenic organism comprising a nucleic acid sequence as claimed in claim 12.

23. The transgenic organism as claimed in claim 22 selected from the group consisting of bacteria, yeasts, fungi, animal and plant organisms.

24. The transgenic organism as claimed in claim 22 selected from the group consisting of bacteria, yeasts, fungi, non-human animal and plant organisms or cells, cell cultures, parts, tissues, organs or propagation material derived therefrom.

25. The transgenic organism as claimed in claim 23 selected from the group of agricultural crop plants.

26. The use of an isolated nucleic acid sequence as claimed in claim 12, wherein the nucleic acid sequence is used in methods for the transgenic expression of nucleic acids or proteins.

27. The transgenic organism as claimed in claim 23, wherein the organism or cell cultures, parts, organs, tissues or transgenic propagation material derived therefrom are used for producing human or animal foods, seeds, pharmaceuticals or fine chemicals.

28. A method for producing human or animal foods, seeds, pharmaceuticals or fine chemicals, where the transgenic organism as claimed in claim 23 is cultured, and the desired human or animal foods, seeds, pharmaceuticals or fine chemical is produced and/or isolated using said organism.

29. A method for producing ketocarotenoids, where the mRNA amount and/or activity of at least one ε-cyclase is reduced by introducing at least one double-stranded RNA as claimed in claim 15.


SEQUENCE LISTING (83 sequences)

SEQ ID NO: 1 - DNA, 312 bp, Tagetes erecta
  promoter (1)..(312)
cgatagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggscaatatagtgatgccacgtagaaaaaggtaagtgaaaacatacaacgtggctttaaaagatggcttggctgctaatcaactcaactcaactcatatcctatccattcaaattcaattcaattctattgaatgcaaa

SEQ ID NO: 2 - DNA, 447 bp, Tagetes erecta
  promoter (1)..(312)
  5′UTR (313)..(444)
  misc_feature (445)..(447): ATG-start codon
cgatagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggscaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaaggttgtttgttgttgttgttgagagacactccaatccaaacagatacaaggcgtgactggatatttctctctcgttcctaacaacagcaacgaagaagaaaaagaatcattactaacaatcaatg

SEQ ID NO: 3 - DNA, 537 bp, Tagetes erecta
  promoter (1)..(312)
  5′UTR (313)..(444)
  sig_peptide (445)..(537)
  CDS (445)..(537): coding for transit peptide of epsilon cyclase
cgatagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggscaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaaggttgtttgttgttgttgttgagagacactccaatccaaacagatacaaggcgtgactggatatttctctctcgttcctaacaacagcaacgaagaagaaaaagaatcattactaacaatcaatgagtatgagagctggacacatgacggcaacaatggcggcttttacatgccctaggtttatgactagcatcagatacacgaagcaaattaag

SEQ ID NO: 4 - protein, 31 aa, Tagetes erecta
MSMRAGHMTATMAAFTCPRFMTSIRYTKQIK

SEQ ID NO: 5 - DNA, 456 bp, Tagetes erecta
  misc_feature (1)..(6): restriction site
ctcgagagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggscaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaaggttgtttgttgttgttgttgagagacactccaatccaaacagatacaaggcgtgactggatatttctctctcgttcctaacaacagcaacgaagaagaaaaagaatcattactaacaatcaatggccatgg

SEQ ID NO: 6 - DNA, 543 bp, Tagetes erecta
  misc_feature (1)..(6): restriction site
ctcgagagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggscaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaaggttgtttgttgttgttgttgagagacactccaatccaaacagatacaaggcgtgactggatatttctctctcgttcctaacaacagcaacgaagaagaaaaagaatcattactaacaatcaatgagtatgagagctggacacatgacggcaacaatggcggcttttacatgccctaggtttatgactagcatcagatacacgaagcaaattaccatgg

SEQ ID NO: 7 - DNA, 1008 bp, Arabidopsis thaliana
gaagatgtcgtggacagttacctagtagaaagccctgaaactcacgatgtcactgacttctgcagcttctacactctcccttcaaccatcctcggtaacccgaactacactacattgaaagctgcgtattcttactacaatgtggccacacagacctcgtttcttcagctgatgaatgatgcgctaattgtctcaaagcaaaagggtttcgatgtgttcaacgcgttggatgtgatgcacaatgagagtttcttgaaagaactgaagtttgggccaggagatggacaacttcattactatctctacaattaccgtttgaaaagtgccttgaagccagcggaactcgggcttgttctcttataagctcaacaacttgatttgatggtatcaacaaacttgaaatttgtctctctttttttttcttcagtctgaactacttctcccatggtttactgaaactgagtttaattattttggcaatgcaattttggattttgcttttagtttcacttgtgttctctgagagagttgaccctgagaagtaaagtcaacactagacgtgtgcgagtttcaatggactgcatttttaggtccaagaagtagctacggcccatctatcgtagaagttgttttttgtactaaagcctgttatagccgaactaggtcgctacgtttcctaacttgtcgtttattaggctacttttaaatcgcacgcaccaatccagttcttacacgcgttttcacaaaaaggaccttacatgggttgttcccccaataagaaaattacacgttgcagaatcgaagcagtggtctatccttgttatcgtcctctcgtctttatggctcttccaagtcattttgagaaattttcgattttgagatttttttccaaatattacaaaaggaaataattagattcctctttctgcttgctataccttgatagaacaatataacaatggtgtaagtcttctcgctgtattcgaaattatttggaggaggaaaatg

SEQ ID NO: 8 - DNA, 1169 bp, Oryza sativa
agaaggagagagagccggccggcgcgacggcggtggcaggagaggtggagagagcccgccgacgcgacggcggcagtggaagaaaaggagaggaggcggccggcgctgtcgaacgcgcacgggaagcagcggacgatgccgagcaggacaatgtcctcctcgtcaccaccttcctcatcgtcgtcgcggtcggtgggtgccgggcggagcggtttttgagggaggggtcggcggccggcgagcccatccgagctcgcctggcttgcgcgagcccgccctcgctgcagcttggtgtgctggggacgacgacgaggaaagggggagagaggaatcaactaccgacgacagcggaggcgggcgagcgcggtgccgggctgctcacgctcgccgaccaggccctcgctccgtcacactcgcctgcctccgttccacccgcagcccgcgccgctccgttcgcagcgctcgcccgtcgccgctccgctgctgtgcagtcccgccgccgcgctcgccctgactgaagaagaaaagagagaagagagaaaagagaagggaaggagaagaaaatagaagaaaaaaatatgtgcagctgatgtatgagccccacatactcttttttaatcttttttgctgactacgatgccacgtcagcgaaaccacctatatatactaccataggatcttgagtgcacggtttatatgagtttaggagtatacatttttagttttatggttaagggatcataaaaaattctcgctattaagttgagtgacgcgcagtgaacttattactcaaacttaacagcgtttgatccattcacatccggcccatagcagcccatatccagcaacgcaccacggtgactgacacgcggggcccaacgaggtggcgccgtggctggtggggcgggtgagtcacgccctcggcgctgcccccaaaagcgaaccatatcctttgcttcgtggggccctgcccaatcgccgcccgccacgtgccccttcccggataccgcctccctccaaacccgctccctctccctctccctcctcctacaatggccgcagcagcagcagccagcagcagacgcagagaccagtagctttcgcagaggcggcagccaccaccgcctcctcctcctcctcctcatccgacggtgtgcaaccaagcgatg

SEQ ID NO: 9 - DNA, 1830 bp, Tagetes erecta
  CDS (141)..(1688): coding for epsilon-cyclase
ggcacgaggcaaagcaaaggttgtttgttgttgttgttgagagacactccaatccaaacagatacaaggcgtgactggatatttctctctcgttcctaacaacagcaacgaagaagaaaaagaatcattactaacaatcaatgagtatgagagctggacacatgacggcaacaatggcggcttttacatgccctaggtttatgactagcatcagatacacgaagcaaattaagtgcaacgctgctaaaagccagctagtcgttaaacaagagattgaggaggaagaagattatgtgaaagccggtggatcggagctgctttttgttcaaatgcaacagaataagtccatggatgcacagtctagcctatcccaaaagctcccaagggtaccaataggaggaggaggagacagtaactgtatactggatttggttgtaattggttgtggtcctgctggccttgctcttgctggagaatcagccaagctaggcttgaatgtcgcacttatcggccctgatcttccttttacaaataactatggtgtttgggaggatgaatttataggtcttggacttgagggctgtattgaacatgtttggcgagatactgtagtatatcttgatgacaacgatcccattctcataggtcgtgcctatggacgagttagtcgtgatttacttcacgaggagttgttgactaggtgcatggagtcaggcgtttcatatctgagctccaaagtggaacggattactgaagctccaaatggcctaagtctcatagagtgtgaaggcaatatcacaattccatgcaggcttgctactgtcgcttctggagcagcttctggaaaacttttgcagtatgaacttggcggtccccgtgtttgcgttcaaacagcttatggtatagaggttgaggttgaaagcataccctatgatccaagcctaatggttttcatggattatagagactacaccaaacataaatctcaatcactagaagcacaatatccaacatttttgtatgtcatgccaatgtctccaactaaagtattctttgaggaaacttgtttggcttcaaaagaggccatgccttttgagttattgaagacaaaactcatgtcaagattaaagactatggggatccgaataaccaaaacttatgaagaggaatggtcatatattccagtaggtggatccttaccaaataccgagcaaaagaaccttgcatttggtgctgctgctagcatggtgcatccagccacaggatattcggttgtaagatcactgtcagaagctcctaattatgcagcagtaattgcaaagattttagggaaaggaaattcaaaacagatgcttgatcatggaagatacacaaccaacatctcaaagcaagcttgggaaacactttggccccttgaaaggaaaagacagagagcattctttctctttggattagcactgattgtccagatggatattgaggggacccgcacattcttccggactttcttccgcttgcccacatggatgtggtgggggtttcttggatcttcgttatcatcaactgacttgataatatttgcgttttacatgtttatcatagcaccgcatagcctgagaatgggtctggttagacatttgctttctgacccgacaggaggaacaatgttaaaagcgtatctcacgatataaataactctagtcgcgatcagtttagattataggcacatcttgcatatatatatgtataaaccttatgtgtgctgtatccttacatcaacacagtcattaattgtatttcttggggtaatgctgatgaagtattttctgg

SEQ ID NO: 10 - protein, 516 aa, Tagetes erecta
MSMRAGHMTATMAAFTCPRFMTSIRYTKQIKCNAAKSQLVVKQEIEEEEDYVKAGGSELLFVQMQQNKSMDAQSSLSQKLPRVPIGGGGDSNCILDLVVIGCGPAGLALAGESAKLGLNVALIGPDLPFTNNYGVWEDEFIGLGLEGCIEHVWRDTVVYLDDNDPILIGRAYGRVSRDLLHEELLTRCMESGVSYLSSKVERITEAPNGLSLIECEGNITIPCRLATVASGAASGKLLQYELGGPRVCVQTAYGIEVEVESIPYDPSLMVFMDYRDYTKHKSQSLEAQYPTFLYVMPMSPTKVFFEETCLASKEAMPFELLKTKLMSRLKTMGIRITKTYEEEWSYIPVGGSLPNTEQKNLAFGAAASMVHPATGYSVVRSLSEAPNYAAVIAKILGKGNSKQMLDHGRYTTNISKQAWETLWPLERKRQRAFFLFGLALIVQMDIEGTRTFFRTFFRLPTWMWWGFLGSSLSSTDLIIFAFYMFIIAPHSLRMGLVRHLLSDPTGGTMLKAYLTI

SEQ ID NO: 11 - DNA, 1916 bp, Tagetes erecta
  CDS (173)..(1747): coding for epsilon-cyclase
attcaaattcaattcaattctattgaatgcaaagcaaagcaaagcaaaggttgtttgttgttgttgtttgagagacactccaatccaaacagatacaaggcgtgactggatatttctctctcgttccttaacaacagcaacgaagaagaaaaagaatcattactcacaatcaatgagtatgagagctggacacatgacggcaacaatggcggcttttacatgccctaggtttatgactagcatcagatacacgaagcaaattaagtgcaacgctgctaaaagccagctagtcgttaaacaagagattgaggaggaagaagattatgtgaaagccggtggatcggagctgctttttgttcaaatgcaacagaataagtccatggatgcacagtctagcctatcccaaaagctcccaagggtaccaataggaggaggaggagacagtaactgtatactggatttggttgtaattggttgtggtcctgctggccttgctcttgctggagaatcagccaagctaggcttgaatgtcgcacttatcggccctgatcttccttttacaaataactatggtgtttgggaggatgaatttataggtcttggacttgagggctgtattgaacatgtttggcgagatactgtagtatatcttgatgacaacgatcccattctcataggtcgtgcctatggacgagttagtcgtgatttacttcacgaggagttgttgactaggtgcatggagtcaggcgtttcatatctgagctccaaagtggaacggattactgaagctccaaatggcctaagtctcatagagtgtgaaggcaatatcacaattccatgcaggcttgctactgtcgcttctggagcagcttctgggaaacttttgcagtatgaacttggcggtccccgtgtttgcgttcaaacagcttatggttacgaggttgaggttgaaagcataccctatgatccaagcctaatggttttcatggattatagagactacaccaaacataaatctcaatcactagaagcacaatatccaacatttttgtatgtcatgccaatgtctccaactaaagtattctttgaggaaacttgtttggcttcaaaagaggccatgccttttgagttattgaagacaaaactcatgtcaagattaaagactatggggatccgaataaccaaaacttatgaagagtatcttgttgcttgtcaatatttggaagaatggtcatatattccagtaggtggatcccttccaaataccgagcaaaagaaccttgcatttggtgctgctgctagcatggtgcatccagccacaggatattcggttgtaagatcactgtcagaagctcctaattatgcagcagtaattgcaaagattttagggaaaggaaattcaaaacagatgcttgatcttggaagatacacaaccaacatctcaaagcaagcttgggaaacactttggccccttgaaaggaaaagacagagagcattctttctctttggattagcactgattgtccagatggatattgaggggacccgcacattcttccggactttcttccgcttgcccacatggatgtggtgggggtttcttggatcttcgttatcatcaactgacttgataatatttgcgttttacatgtttatcatagcaccgcatagcctgagaatgggtctggttagacatttgctttctgacccgacaggaggaacaatgttaaaagcgtatctcacgatataaataactctagtcgcgatcagtttagattataggcacatcttgcatatatatatgtataaaccttatgtgtgctgtatccttacatcaacacagtcattaattgtatttcttggggtaatgctgatgaagtattttctggaaaaaaaaaaaaaaaaaaaaaaaaaaa

SEQ ID NO: 12 - protein, 525 aa, Tagetes erecta
MSMRAGHMTATMAAFTCPRFMTSIRYTKQIKCNAAKSQLVVKQEIEEEEDYVKAGGSELLFVQMQQNKSMDAQSSLSQKLPRVPIGGGGDSNCILDLVVIGCGPAGLALAGESAKLGLNVALIGPDLPFTNNYGVWEDEFIGLGLEGCIEHVWRDTVVYLDDNDPILIGRAYGRVSRDLLHEELLTRCMESGVSYLSSKVERITEAPNGLSLIECEGNITIPCRLATVASGAASGKLLQYELGGPRVCVQTAYGYEVEVESIPYDPSLMVFMDYRDYTKHKSQSLEAQYPTFLYVMPMSPTKVFFEETCLASKEAMPFELLKTKLMSRLKTMGIRITKTYEEYLVACQYLEEWSYIPVGGSLPNTEQKNLAFGAAASMVHPATGYSVVRSLSEAPNYAAVIAKILGKGNSKQMLDLGRYTTNISKQAWETLWPLERKRQRAFFLFGLALIVQMDIEGTRTFFRTFFRLPTWMWWGFLGSSLSSTDLIIFAFYMFIIAPHSLRMGLVRHLLSDPTGGTMLKAYLTI

SEQ ID NO: 13 - DNA, 1818 bp, Arabidopsis thaliana
  CDS (104)..(1675): coding for epsilon-cyclase
aggaaataattagattcctctttctgcttgctataccttgatagaacaatataacaatggtgtaagtcttctcgctgtattcgaaattatttggaggaggaaaatggagtgtgttggggctaggaatttcgcagcaatggcggtttcaacatttccgtcatggagttgtcgaaggaaatttccagtggttaagagatacagctataggaatattcgtttcggtttgtgtagtgtcagagctagcggcggcggaagttccggtagtgagagttgtgtagcggtgagagaagatttcgctgacgaagaagattttgtgaaagctggtggttctgagattctatttgttcaaatgcagcagaacaaagatatggatgaacagtctaagcttgttgataagttgcctcctatatcaattggtgatggtgctttggatctagtggttattggttgtggtcctgctggtttagccttggctgcagaatcagctaagcttggattaaaagttggactcattggtccagatcttccttttactaacaattacggtgtttgggaagatgaattcaatgatcttgggctgcaaaaatgtattgagcatgtttggagagagactattgtgtatctggatgatgacaagcctattaccattggccgtgcttatggaagagttagtcgacgtttgctccatgaggagcttttgaggaggtgtgtcgagtcaggtgtctcgtaccttagctcgaaagttgacagcataacagaagcttctgatggccttagacttgttgcttgtgacgacaataacgtcattccctgcaggcttgccactgttgcttctggagcagcttcgggaaagctcttgcaatacgaagttggtggacctagagtctgtgtgcaaactgcatacggcgtggaggttgaggtggaaaatagtccatatgatccagatcaaatggttttcatggattacagagattatactaacgagaaagttcggagcttagaagctgagtatccaacgtttctgtacgccatgcctatgacaaagtcaagactcttcttcgaggagacatgtttggcctcaaaagatgtcatgccctttgatttgctaaaaacgaagctcatgttaagattagatacactcggaattcgaattctaaagacttacgaagaggagtggtcctatatcccagttggtggttccttgccaaacaccgaacaaaagaatctcgcctttggtgctgccgctagcatggtacatcccgcaacaggctattcagttgtgagatctttgtctgaagctccaaaatatgcatcagtcatcgcagagatactaagagaagagactaccaaacagatcaacagtaatatttcaagacaagcttgggatactttatggccaccagaaaggaaaagacagagagcattctttctctttggtcttgcactcatagttcaattcgataccgaaggcattagaagcttcttccgtactttcttccgccttccaaaatggatgtggcaagggtttctaggatcaacattaacatcaggagatctcgttctctttgctttatacatgttcgtcatttcaccaaacaatttgagaaaaggtctcatcaatcatctcatctctgatccaaccggagcaaccatgataaaaacctatctcaaagtatgatttacttatcaactcttaggtttgtgtatatatatgttgatttatctgaataatcgatcaaagaatggtatgtgggttactaggaagttggaaacaaacatgtatagaatctaaggagtgatcgaaatggagatggaaac

SEQ ID NO: 14 - protein, 524 aa, Arabidopsis thaliana
MECVGARNFAAMAVSTFPSWSCRRKFPVVKRYSYRNIRFGLCSVRASGGGSSGSESCVAVREDFADEEDFVKAGGSEILFVQMQQNKDMDEQSKLVDKLPPISIGDGALDLVVIGCGPAGLALAAESAKLGLKVGLIGPDLPFTNNYGVWEDEFNDLGLQKCIEHVWRETIVYLDDDKPITIGRAYGRVSRRLLHEELLRRCVESGVSYLSSKVDSITEASDGLRLVACDDNNVIPCRLATVASGAASGKLLQYEVGGPRVCVQTAYGVEVEVENSPYDPDQMVFMDYRDYTNEKVRSLEAEYPTFLYAMPMTKSRLFFEETCLASKDVMPFDLLKTKLMLRLDTLGIRILKTYEEEWSYIPVGGSLPNTEQKNLAFGAAASMVHPATGYSVVRSLSEAPKYASVIAEILREETTKQINSNISRQAWDTLWPPERKRQRAFFLFGLALIVQFDTEGIRSFFRTFFRLPKWMWQGFLGSTLTSGDLVLFALYMFVISPNNLRKGLINHLISDPTGATMIKTYLKV

SEQ ID NO: 15 - DNA, 1623 bp, Oryza sativa
  CDS (1)..(1620): coding for epsilon-cyclase
atggagttctccggcggcgcgaccgtgtcggcgccgttcggttgctgccgtgcggcgtggggcgccgcggcggcgggtgcgggggcggagggaaggagcaggagggttgtgccgcgcgcggtggagccgcggcggcgcgggcggtggatggtgaggtgcgtggcgacggagaagcacaaggacgcggcggcgcggcgcggcggcgtggaggtggagttcgccgacgaggaggactacgtcaagggcggcggcggcgagcttctctacgtgcaaatgcaggcgtccaagtccatggacagccagtccaagatctcctccaagctgctgcccatacccgatgaaaattcagttcttgatttggtcatcattggctgcggtccagctggcttatccctagcagcagagtcagctaagaaagggctcaatgttggtctcattggccctgatcttccattcacgaacaactacggtgtgtgggaggatgaattcaaagacctgggcctggagagctgcattgaacatgtctggaaggatactatcgtgtacctagacggtaacaagccaataatgattggccgtgcgtatggcagggttcacagggacttgctgcacgaggagttgctgagacgatgctatgacgctggcgtcacatacctcagctcgaaggtggacaagatcatggaatctcctgatggacatcgggtagtctgttgtgaaggggatcgtgaggtactttgcaggcttgccattgttgcatctggggcagcatctggtaggcttctagagtacgaggttggtggtccgcgtgtttgtgtgcagactgcatatggtgtcgaagtcgaggtggaaaacaatccatatgatcccagcttaatggttttcatggactacagagattgcttcaaagacaaattctcacatcctgagcaaggaaatccaacgttcctctatgccatgcccatgtcatccacacgaattttctttgaggaaacatgcctagcttctaaagaagcaatgccctttgacctccttaaaaagcggttgatgtctcggttggatgcaatgggagttcatattcgaaaagtatacgaggaggaatggtcctacattcctgttggagggtccttaccaaatacagaccagaaaaatctcgcatttggtgcggcagcaagtatggtgcatcctgcaaccggatactcggtggttagatcattgtctgaagctccaagatatgcatctgtgatatctgatatcttgagaaaccgtgtctaccctggagaatatttgcctggaacctctcaaagttccagtccatcaatgcttgcatggagaacattatggccccaagaacggaaacgtcaacgatcattcttcctttttgggctggctttgataatccaactgaataacgaaggcattcagacattctttgaaacctttttccggttgcccaaatggatgtggcgaggattccttggttcgacgctttcttcagtggatctcatactctttgcattctacatgttcacaattgcgccgaaccaaatgcgaatgaaccttgtcagacatctcctctctgatccgaccggctcaacgatgatcaagacctacctgaccttgtaa

SEQ ID NO: 16 - protein, 540 aa, Oryza sativa
MEFSGGATVSAPFGCCRAAWGAAAAGAGAEGRSRRVVPRAVEPRRRGRWMVRCVATEKHKDAAARRGGVEVEFADEEDYVKGGGGELLYVQMQASKSMDSQSKISSKLLPIPDENSVLDLVIIGCGPAGLSLAAESAKKGLNVGLIGPDLPFTNNYGVWEDEFKDLGLESCIEHVWKDTIVYLDGNKPIMIGRAYGRVHRDLLHEELLRRCYDAGVTYLSSKVDKIMESPDGHRVVCCEGDREVLCRLAIVASGAASGRLLEYEVGGPRVCVQTAYGVEVEVENNPYDPSLMVFMDYRDCFKDKFSHPEQGNPTFLYAMPMSSTRIFFEETCLASKEAMPFDLLKKRLMSRLDAMGVHIRKVYEEEWSYIPVGGSLPNTDQKNLAFGAAASMVHPATGYSVVRSLSEAPRYASVISDILRNRVYPGEYLPGTSQSSSPSMLAWRTLWPQERKRQRSFFLFGLALIIQLNNEGIQTFFETFFRLPKWMWRGFLGSTLSSVDLILFAFYMFTIAPNQMRMNLVRHLLSDPTGSTMIKTYLTL

SEQ ID NO: 17 - protein, 10 aa, Artificial sequence
  Description of the artificial sequence protein motif for epsilon-cyclase
GGGPAGLAVA

SEQ ID NO: 18 - protein, 8 aa, Artificial sequence
  Description of the artificial sequence protein motif for epsilon-cyclase
LNRXYGKV

SEQ ID NO: 19 - protein, 8 aa, Artificial sequence
  Description of the artificial sequence protein motif for epsilon-cyclase
MVFMDYRD

SEQ ID NO: 20 - protein, 8 aa, Artificial sequence
  Description of the artificial sequence protein motif for epsilon-cyclase
PTFLYAMP

SEQ ID NO: 21 - protein, 14 aa, Artificial sequence
  Description of the artificial sequence protein motif for epsilon-cyclase
AXMVHPSTGYMVAR

SEQ ID NO: 22 - protein, 13 aa, Artificial sequence
  Description of the artificial sequence protein motif for epsilon-cyclase
LWPXERRRQRXFF

SEQ ID NO: 23 - DNA, 1780 bp, Lactuca sativa
  CDS (77)..(1675): coding for epsilon-cyclase
gaaacaaatgacgtgaaagttcttcaaaattgaattaattgtaatcctgaaaacttgatttgtgatagaagaatcaatggagtgctttggagctcgaaacatgacggcaacaatggcggtttttacgtgccctagattcacggactgtaatatcaggcacaaattttcgttactgaaacaacgaagatttactaatttatcagcatcgtcttcgttgcgtcaaattaagtgcagcgctaaaagcgaccgttgtgtagtggataaacaagggatttccgtagcagacgaagaagattatgtgaaggccggtggatcggagctgttttttgttcaaatgcagcggactaagtccatggaaagccagtctaaactttccgaaaagctagcacagataccaattggaaattgcatacttgatctggttgtaatcggttgtggccctgctggccttgctcttgctgcagagtcagccaaactagggttgaacgttggactcattggccctgatcttccttttacaaacaattatggtgtttggcaggatgaatttataggtcttggacttgaaggatgcattgaacattcttggaaagatactcttgtataccttgatgatgctgatcccatccgcataggtcgtgcatatggcagagttcatcgtgatttacttcatgaagagttgttaagaaggtgtgtggaatcaggtgtttcatatctaagctccaaagtagaaagaatcactgaagctccaaatggctatagtctcattgaatgtgaaggcaatatcaccattccatgcaggcttgctactgttgcatcaggggcagcttcagggaaatttctggagtatgaacttgggggtccccgtgtttgtgtccaaacagcttatggtatagaggttgaggttgaaaacaacccctatgatccagatctaatggtgttcatggattatagagacttctcaaaacataaaccggaatctttagaagcaaaatatccgactttcctctatgtcatggccatgtctccaacaaaaatattcttcgaggaaacttgtttagcttcaagagaagccatgcctttcaatcttctaaagtccaaactcatgtcacgattaaaggcaatgggtatccgaataacaagaacgtacgaagaggaatggtcgtatatccccgtaggtggatcgttacctaatacagaacaaaagaatctcgcatttggtgctgcagctagtatggtgcaccctgccacagggtattcagttgttcgatctttgtcagaagctcctaattatgcagcagtcattgctaagattttaagacaagatcaatctaaagagatgatttctcttggaaaatacactaacatttcaaaacaagcatgggaaacattgtggccacttgaaaggaaaagacagcgagccttctttctattcggactatcacacatcgtgctaatggatctagagggaacacgtacatttttccgtactttctttcgtttgcccaaatggatgtggtggggatttttggggtcttctttatcttcaacggatttgataatatttgcgctttatatgtttgtgatagcacctcacagcttgagaatggaactggttagacatctactttctgatccgacaggggcaactatggtaaaagcatatctcactatatagatttagattatataaataatacccatatcttgcatatatataagccttatttatttcttttgtatccttacaacaacatactcgttaattatatgtttttta

SEQ ID NO: 24 - protein, 533 aa, Lactuca sativa
MECFGARNMTATMAVFTCPRFTDCNIRHKFSLLKQRRFTNLSASSSLRQIKCSAKSDRCVVDKQGISVADEEDYVKAGGSELFFVQMQRTKSMESQSKLSEKLAQIPIGNCILDLVVIGCGPAGLALAAESAKLGLNVGLIGPDLPFTNNYGVWQDEFIGLGLEGCIEHSWKDTLVYLDDADPIRIGRAYGRVHRDLLHEELLRRCVESGVSYLSSKVERITEAPNGYSLIECEGNITIPCRLATVASGAASGKFLEYELGGPRVCVQTAYGIEVEVENNPYDPDLMVFMDYRDFSKHKPESLEAKYPTFLYVMAMSPTKIFFEETCLASREAMPFNLLKSKLMSRLKAMGIRITRTYEEEWSYIPVGGSLPNTEQKNLAFGAAASMVHPATGYSVVRSLSEAPNYAAVIAKILRQDQSKEMISLGKYTNISKQAWETLWPLERKRQRAFFLFGLSHIVLMDLEGTRTFFRTFFRLPKWMWWGFLGSSLSSTDLIIFALYMFVIAPHSLRMELVRHLLSDPTGATMVKAYLTI

SEQ ID NO: 25 - DNA, 1848 bp, Adonis palaestina
  CDS (116)..(1702): coding for epsilon cyclase
gagagaaaaagagtgttatattaatgttactgtcgcattcttgcaacacatattcagactccattttcttgttttctcttcaaaacaacaaactaatgtgacggagtatctagctatggaactacttggtgttcgcaacctcatctcttcttgccctgtctggacttttggaacaagaaaccttagtagttcaaaactagcttataacatacatcgatatggttcttcttgtagagtagattttcaagtgagggctgatggtggaagcgggagtagaacttctgttgcttataaagagggttttgtggacgaggaggattttatcaaagctggtggttctgagcttttgtttgtccaaatgcagcaaacaaagtctatggagaaacaggccaagctcgccgataagttgccaccaatacctttcggagaatctgtgatggacttggttgtaataggttgtggacctgctggtctttcactggctgcagaagctgctaagctaggcttgaaagttggccttattggtcctgatcttccttttacaaataattatggtgtgtgggaagacgagttcaaagatcttggacttgaacgttgtatcgagcatgcttggaaggacaccatcgtatatcttgacaatgatgctcctgtccttattggtcgtgcatatggacgagttagccggcatttgctgcatgaagagttgctgaaaaggtgtgtcgagtcaggtgtatcatatctgaattctaaagtggaaaggatcactgaagctggtgatggccatagtcttgtagtttgtgaaaacgacatctttatcccttgcaggcttgctactgttgcatctggagcagcttcagggaaacttttggagtatgaagtaggtggccctcgtgtttgtgtccaaactgcttatggtgtggaggttgaggtggagaacaatccatacgatcccaacttaatggtatttatggactacagagactatatgcaacagaaattacagtgctcggaagaagaatatccaacatttctctatgtcatgcccatgtcgccaacaagacttttttttgaggaaacctgtttggcctcaaaagatgccatgcctttcgatctactgaagagaaaactaatgtcacgattgaagactctgggtatccaagttacaaaaatttatgaagaggaatggtcttatattcctgttgggggttctttaccaaacacagagcaaaagaacctagcatttggtgctgcagcaagcatggtgcatccagcaacaggctattcggttgtacgatcactatcagaagctccaaaatatgcttctgtaattgcaaagattttgaagcaagataactctgcatatgtggtttctggacaaagcagtgcagtaaacatttcaatgcaagcatggagcagtctttggccaaaggagcgaaaacgtcaaagagcattctttcttttcgggttagagcttattgtgcagctagatattgaagcaaccagaacgttctttagaaccttcttccgcttgccaacttggatgtggtggggtttccttgggtcttcactatcatctttcgatcttgtattgttttccatgtacatgtttgttttggccccgaacagcatgaggatgtcacttgtgagacatttgctttcagatccttctggtgcagttatggttaaagcttacctcgaaaggtaatctgttttatgaaactatagtgtctcattaaataaatgaggatccttcgtatatgtatatgatcatctctatgtatatcctatattctaatctcataaagtaatcgaaaattcattgatagaaaaaaaaaaaaaaaaaaaaaa

SEQ ID NO: 26 - protein, 529 aa, Adonis palaestina
MELLGVRNLISSCPVWTFGTRNLSSSKLAYNIHRYGSSCRVDFQVRADGGSGSRTSVAYKEGFVDEEDFIKAGGSELLFVQMQQTKSMEKQAKLADKLPPIPFGESVMDLVVIGCGPAGLSLAAEAAKLGLKVGLIGPDLPFTNNYGVWEDEFKDLGLERCIEHAWKDTIVYLDNDAPVLIGRAYGRVSRHLLHEELLKRCVESGVSYLNSKVERITEAGDGHSLVVCENDIFIPCRLATVASGAASGKLLEYEVGGPRVCVQTAYGVEVEVENNPYDPNLMVFMDYRDYMQQKLQCSEEEYPTFLYVMPMSPTRLFFEETCLASKDAMPFDLLKRKLMSRLKTLGIQVTKIYEEEWSYIPVGGSLPNTEQKNLAFGAAASMVHPATGYSVVRSLSEAPKYASVIAKILKQDNSAYVVSGQSSAVNISMQAWSSLWPKERKRQRAFFLFGLELIVQLDIEATRTFFRTFFRLPTWMWWGFLGSSLSSFDLVLFSMYMFVLAPNSMRMSLVRHLLSDPSGAVMVKAYLER

SEQ ID NO: 27 - DNA, 1898 bp, Adonis palaestina
  CDS (113)..(1699): coding for epsilon-cyclase
aaaggagtgttctattaatgttactgtcgcattcttgcaacacttatattcaaactccattttcttcttttctcttcaaaacaacaaactaatgtgagcagagtatctggctatggaactacttggtgttcgcaacctcatctcttcttgccctgtgtggacttttggaacaagaaaccttagtagttcaaaactagcttataacatacatcgatatggttcttcttgtagagtagattttcaagtgagagctgatggtggaagcgggagtagaagttctgttgcttataaagagggttttgtggatgaagaggattttatcaaagctggtggttctgagcttttgtttgtccaaatgcagcaaacaaagtctatggagaaacaggccaagctcgccgataagttgccaccaataccttttggagaatccgtgatggacttggttgtaataggttgtggacctgctggtctttcactggctgcagaagctgctaagctagggttgaaagttggccttattggtcctgatcttccttttacaaataattatggtgtgtgggaagacgagttcaaagatcttggacttgaacgttgtatcgagcatgcttggaaggacaccatcgtatatcttgataatgatgctcctgtccttattggtcgtgcatatggacgagttagtcgacatttgctacatgaggagttgctgaaaaggtgtgtggagtcaggtgtatcatatctggattctaaagtggaaaggatcactgaagctggtgatggccatagccttgtagtttgtgaaaatgagatctttatcccttgcaggcttgctactgttgcatctggagcagcttcagggaaacttttggagtatgaagtaggtggccctcgtgtttgtgtccaaaccgcttatggggtggaggttgaggtggagaacaatccatacgatcccaacttaatggtattcatggactacagagactatatgcaacagaaattacagtgctcggaagaagaatatccaacatttctctatgtcatgcccatgtcgccaacaagacttttttttgaggaaacctgtttggcctcaaaagatgccatgccattcgatctactgaagagaaaactgatgtcacgattgaagactctgggtatccaagttacaaaagtttatgaagaggaatggtcatatattcctgttggtggttctttaccaaacacagagcaaaagaacctagcatttggtgctgcagcaagcatggtgcatccagcaacaggctattcggttgtacggtcactgtcagaagctccaaaatatgcttctgtaattgcaaagattttgaagcaagataactctgcgtatgtggtttctggacaaagtagtgcagtaaacatttcaatgcaagcatggagcagtctttggccaaaggagcgaaaacgtcaaagagcattctttctttttggattagagcttattgtgcagctagatattgaagcaaccagaacattctttagaaccttcttccgcttgccaacttggatgtggtggggtttccttgggtcttcactatcatctttcgatctcgtcttgttttccatgtacatgtttgttttggcgccaaacagcatgaggatgtcacttgtgagacatttgctttcagatccttctggtgcagttatggtaagagcttacctcgaaaggtagtctcatctattattaaactctagtgtttcaccaaataaatgaggatccttcgaatgtgtatatgatcatctctatgtatatcctgtactctaatctcataaagtaaatgccgggtttgatattgttgtgtcaaaccggccaatgatataaagtaaatttattgatacaaaagtagtttttttccttaaaaaaaaaa

SEQ ID NO: 28 - protein, 529 aa, Adonis palaestina
MELLGVRNLISSCPVWTFGTRNLSSSKLAYNIHRYGSSCRVDFQVRADGGSGSRSSVAYKEGFVDEEDFIKAGGSELLFVQMQQTKSMEKQAKLADKLPPIPFGESVMDLVVIGCGPAGLSLAAEAAKLGLKVGLIGPDLPFTNNYGVWEDEFKDLGLERCIEHAWKDTIVYLDNDAPVLIGRAYGRVSRHLLHEELLKRCVESGVSYLDSKVERITEAGDGHSLVVCENEIFIPCRLATVASGAASGKLLEYEVGGPRVCVQTAYGVEVEVENNPYDPNLMVFMDYRDYMQQKLQCSEEEYPTFLYVMPMSPTRLFFEETCLASKDAMPFDLLKRKLMSRLKTLGIQVTKVYEEEWSYIPVGGSLPNTEQKNLAFGAAASMVHPATGYSVVRSLSEAPKYASVIAKILKQDNSAYVVSGQSSAVNISMQAWSSLWPKERKRQRAFFLFGLELIVQLDIEATRTFFRTFFRLPTWMWWGFLGSSLSSFDLVLFSMYMFVLAPNSMRMSLVRHLLSDPSGAVMVRAYLER

SEQ ID NO: 29 - DNA, 1661 bp, Arabidopsis thaliana
  CDS (2)..(1501): coding for epsilon-cyclase
gatggatactctgttgaaaacacccaacaagctcgattttttcatccctcagtttcatgggtttgagagattatgcagtaacaatccatacccttcaagggttaggcttggtgtgaagaaaagggctatcaaaattgtctctagtgtagtgagtggtagcgctgctcttttggatcttgttcctgaaactaagaaggagaatcttgactttgagcttcctttgtacgacacttccaagagtcaagttgttgatttggctattgttggtggtggtcctgctggtttagccgtggctcagcaggtttctgaagctggactctctgtttgttccattgatccttctcctaagctcatatggcctaacaattatggagtttgggttgatgagtttgaggctatggatttactagactgcctggataccacatggtctggtgctgttgtctatgtcgatgaaggtgtcaagaaggatttgagccggccttatgggagagttaaccggaaacagctcaaatccaaaatgcttcagaaatgtattaccaacggtgttaaatttcatcagtctaaggtcactaatgtggttcacgaggaggcaaactccactgtggtctgcagtgacggtgtaaagattcaggcttccgtggttcttgatgccactgggttttcccgatgcttggttcagtatgacaaaccttacaaccctgggtaccaagtagcttacgggattatagctgaagttgatggtcacccattcgatgtagacaaaatggtgttcatggattggagagacaaacatctggactcatatcctgagctgaaagaacggaacagcaagatcccaacgttcttgtacgctatgccattttcttccaaccgaatatttcttgaagaaacttctttagttgctagacctggtctgagaatggaagatatccaagaaagaatggctgctagactgaaacatctggggatcaatgtgaagaggattgaggaagacgagcgttgtgtgatcccgatgggcggtcctttaccagtcttacctcaacgggttgtggggattggtgggacagcaggaatggttcatccttcaactggttacatggttgctaggactcttgcagctgcaccaatagttgcaaatgccattgtgagatacctcggttcaccaagtagtaatagcctgagaggagatcaactctctgctgaggtttggagagacttgtggcctatcgaacggcgtagacagagggagttcttctgttttggaatggatattctgctgaaactcgatttagacgctactagaaggttctttgatgcattctttgatctgcaacctcattactggcacggattcttgtcttccaggctgtttctcccggaactgttggtcttcgggttgtcgctcttctcacacgcttccaatacctcaagattggagatcatgacaaaggggactgttcctcttgctaagatgatcaacaatttggtacaagatagagactaaggaccagaaacttagacatataagtatatctgttctttggttcttgaccagtagtatatccgcattgcaagtcgttggataattgtgtataaaccacagatccataacctgaatccttgtgaaatcaaattgttactactagttcattaaaacc

SEQ ID NO: 30 - protein, 500 aa, Arabidopsis thaliana
MDTLLKTPNKLDFFIPQFHGFERLCSNNPYPSRVRLGVKKRAIKIVSSVVSGSAALLDLVPETKKENLDFELPLYDTSKSQVVDLAIVGGGPAGLAVAQQVSEAGLSVCSIDPSPKLIWPNNYGVWVDEFEAMDLLDCLDTTWSGAVVYVDEGVKKDLSRPYGRVNRKQLKSKMLQKCITNGVKFHQSKVTNVVHEEANSTVVCSDGVKIQASVVLDATGFSRCLVQYDKPYNPGYQVAYGIIAEVDGHPFDVDKMVFMDWRDKHLDSYPELKERNSKIPTFLYAMPFSSNRIFLEETSLVARPGLRMEDIQERMAARLKHLGINVKRIEEDERCVIPMGGPLPVLPQRVVGIGGTAGMVHPSTGYMVARTLAAAPIVANAIVRYLGSPSSNSLRGDQLSAEVWRDLWPIERRRQREFFCFGMDILLKLDLDATRRFFDAFFDLQPHYWHGFLSSRLFLPELLVFGLSLFSHASNTSRLEIMTKGTVPLAKMINNLVQDR

SEQ ID NO: 31 - DNA, 1550 bp, Citrus X paradisi
  CDS (45)..(1355): coding for epsilon-cyclase
aaataaaaaacactggcaaatgctctattagtacagtgttattaatggacatgtttatactaccgccaatatcaattggtaatggtattttggatttggtggtgattggttgtggcccagctggtcttgctttggctgcagaatcagcgaagttgggattaaatgttggacttattggcccggatctccctttcacaaacaattatggtgtgtgggaagatgaatttagagatcttggacttgaagggtgtatcgaacatgtctggagagacacagttgtatatattgatgaagatgaacccatcttgattggtcgtgcttatggacgagttagtcgacatttgcttcatgaagaattattaagaaggtgtgtcgagtcaggtgtttcatatcttagctcaaaagtggaaagcattacggaatctaccagtggtcatcgtcttgtagcttgtgaacatgatatgattgtcccctgcaggcttgctactgttgcttctggagcagcatcagggaagctattggaatatgaggtggggggtcccaaagtttctgtccaaacagcttatggtgtggaggttgaggtggaaaataatccatatgatccaagccttatggttttcatggactacagagactgtactaagcaagaagttccatcttttgaatctgacaatccaacatttctttatgtcatgcccatgtcttcaacaagagttttctttgaggaaacttgtttggcatcgaaagatggtttacgttttgacatattgaagaaaaagctcatggcaaggttagagagattgggaatccaggttttgaaaacttatgaagaggaatggtcatatattccagttggtggttccttaccaaatacagaacaaagaaacctcgcatttggtgctgctgctagcatggtgcatccagccactggctactcagtagtcagatcactgtcagaggctccaaactatgcttctgcaattgcatatatattgaaacacgatcattccagaggtagacttacacatgaacaaagtaatgagaatatctcaatgcaagcttggaatactctctggccacaggaaaggaagcgccaaagagcttttttcctctttggactagcactcattttgcaactggatattgagggcatcaggacattctttcgcactttcttccgattacccaagtggatgtggcacggtttccttggttctagtctctcatcagccgatctcattctatttgccttctatatgtttattatagcaccaaatgatctgagaaagtgccttatcagacatctagtttcagatccaactggagcaactatggtaagaacatacctgactttatagttagtttgtattttccatatttcagcccttgtttggtatattttggattgccatacgtgacacataatgagcttgtatatatactccatgtatactgtaaactgttagtttgacaaatgaagcccttttttatttttaaaaaaaaaaaaaaaaaaaaaaaaaaaaaaaaaaaaaaaaaaaaaaaaaaaaaaa

SEQ ID NO: 32 - protein, 437 aa, Citrus X paradisi
MDMFILPPISIGNGILDLVVIGCGPAGLALAAESAKLGLNVGLIGPDLPFTNNYGVWEDEFRDLGLEGCIEHVWRDTVVYIDEDEPILIGRAYGRVSRHLLHEELLRRCVESGVSYLSSKVESITESTSGHRLVACEHDMIVPCRLATVASGAASGKLLEYEVGGPKVSVQTAYGVEVEVENNPYDPSLMVFMDYRDCTKQEVPSFESDNPTFLYVMPMSSTRVFFEETCLASKDGLRFDILKKKLMARLERLGIQVLKTYEEEWSYIPVGGSLPNTEQRNLAFGAAASMVHPATGYSVVRSLSEAPNYASAIAYILKHDHSRGRLTHEQSNENISMQAWNTLWPQERKRQRAFFLFGLALILQLDIEGIRTFFRTFFRLPKWMWHGFLGSSLSSADLILFAFYMFIIAPNDLRKCLIRHLVSDPTGATMVRTYLTL

SEQ ID NO: 33 - DNA, 1830 bp, Citrus X paradisi
  CDS (89)..(1660): coding for epsilon-cyclase
gggttcagtttgtcgttgaggacaggccacaaacgcaacacaagcttcatctttaccaaatttccgtaagcaacttctgggctgaaaaatgctcccatttctctcctctctgcttaatggagtcacggataacccttgtaggaaagccatggatactttactcaaaactcataacaagcttgaattcttgccccaagttcacggggctttggaaaaatccagtagtttaagctcattgaagattcagaaccaggagcttaggtttggtctcaagaagtctcgtcaaaagaggaataggagttgtttcattaaggctagtagtagtgctcttttggagctagttcctgaaaccaagaaggaaaatcttgaatttgagcttcccatgtatgacccatcaaagggccttgttgtagacctagcagttgtcggtggcggcccggctgggcttgctgttgctcagcaagtttcaggggcggggctttcggtttgctcgattgatccatctcccaaattgatttggccaaataattatggtgtttgggtggatgaatttgaggccatggatttgcttgattgccttgatactacttggtctggtgctgttgtgcacattgatgataatacaaagaaggatcttaatagaccttatgggagagttaataggaagttgctgaagtcgaaaatgctgcaaaaatgcataaccaatggtgttaagtttcaccaagctaaagttattaaggttattcatgaagagtccaaatctttgttgatttgcaatgatggtgtgacaattcaggcagccgtggttcttgatgctacggggttctctaggtgtcttgtccagtatgataagccctataatccaggttaccaagtggcatatggaatactagctgaggtagaacagcacccgtttgatttagacaagatggttttcatggattggagagattcgcatctgaacaacaattcgcagctcaaagaggcaaatagcaaaattcctacttttctttatgccatgcccttttcgtcaaacaggatatttcttgaagagacttcgctagtggcgcggcctggagtgccaatgaaagatatccaggaaagaatggtggctagattaaagcacttaggcataaaagttaaaagcattgaagaggatgagcattgtgtcattccgatgggtgggccccttccagtgcttcctcaaagagttgttggaataggtggtaccgctgggatggtgcacccttcaactggctatatggtggcaaggactttagctgcggctcctattgttgcaaatgcaatcgttcgaagcctcagttctgacagaagcatttcaggacacaaattgtctgctgaagtttggaaagatttgtggcccatagaaaggagaaggcaaagggagttcttctgttttggtatggatatcctgctcaaacttgacttacctgccactaggaggtttttcgatgctttttttgatctggagcctcgttattggcatggtttcttatcatcgagattgtttctccccgagcttttagtttttgggctttctctattctcacatgcctctaatacttctaggctagagatcatggcaaagggaactcttcctttggttaacatgatcaacaacttggtacaagatacagattaaggtgaccatgatagttataatgtgcttaataactcatgcactaatcgtttataaaacacttcaaattagttttgatgtttatagcttattacatgaaccaaagcttatgatagacgtgctttggtatttaagagtttcagccaaaaaaaaaaaaaaaaaaaaaaaaa

SEQ ID NO: 34 - protein, 524 aa, Citrus X paradisi
MLPFLSSLLNGVTDNPCRKAMDTLLKTHNKLEFLPQVHGALEKSSSLSSLKIQNQELRFGLKKSRQKRNRSCFIKASSSALLELVPETKKENLEFELPMYDPSKGLVVDLAVVGGGPAGLAVAQQVSGAGLSVCSIDPSPKLIWPNNYGVWVDEFEAMDLLDCLDTTWSGAVVHIDDNTKKDLNRPYGRVNRKLLKSKMLQKCITNGVKFHQAKVIKVIHEESKSLLICNDGVTIQAAVVLDATGFSRCLVQYDKPYNPGYQVAYGILAEVEQHPFDLDKMVFMDWRDSHLNNNSQLKEANSKIPTFLYAMPFSSNRIFLEETSLVARPGVPMKDIQERMVARLKHLGIKVKSIEEDEHCVIPMGGPLPVLPQRVVGIGGTAGMVHPSTGYMVARTLAAAPIVANAIVRSLSSDRSISGHKLSAEVWKDLWPIERRRQREFFCFGMDILLKLDLPATRRFFDAFFDLEPRYWHGFLSSRLFLPELLVFGLSLFSHASNTSRLEIMAKGTLPLVNMINNLVQDTD

SEQ ID NO: 35 - DNA, 787 bp, Citrus sinensis
  CDS (2)..(787): coding for epsilon-cyclase  (partial)
tcttgctttggctgcagaatcagcgaagttgggattaaatgttggacttattggcccggatctccctttcacaaacaattatggtgtgtgggaagatgaatttagagatcttggacttgaagggtgtatcgaacaagtctggagagacacagttgtatatattgatgaagatgaacccatcttgattggtcgtgcttatggacgagttagtcgacatttgcttcatgaagaattattaagaaggtgtgtcgagtcaggtgtatcatatcttagctcaaaagtggaaagcattacggaatctaccagtggtcatcgtcttgtagcttgtgaacatgatatgattgtcccctgcaggcttgctactgttgcttctggagcagcatcagggaagctattggaatatggggtggggggtcccaaagtttctgtccaaacagcttatggtgtggaggttgaggtggaaaataatccatatgatccaagccttatggttttcatggactacagagactgtactaagcaagaagttccatcttttgaatctgacaatccaacatttctttatgtcatgcccatgtcttcaacaagagttttctttgaggaaacttgtttggcatcgaaagatggcttacgttttgacatattgaagaaaaagctcatggcaaggttagagagattgggaatccaggttttgaaaacttatgaagaggaatggtcatatattccagttggtggttccttaccaaatacagaacaaagaaacctcgcatatggtgctgctgctagc

SEQ ID NO: 36 - protein, 262 aa, Citrus sinensis
LALAAESAKLGLNVGLIGPDLPFTNNYGVWEDEFRDLGLEGCIEQVWRDTVVYIDEDEPILIGRAYGRVSRHLLHEELLRRCVESGVSYLSSKVESITESTSGHRLVACEHDMIVPCRLATVASGAASGKLLEYGVGGPKVSVQTAYGVEVEVENNPYDPSLMVFMDYRDCTKQEVPSFESDNPTFLYVMPMSSTRVFFEETCLASKDGLRFDILKKKLMARLERLGIQVLKTYEEEWSYIPVGGSLPNTEQRNLAYGAAAS

SEQ ID NO: 37 - DNA, 2357 bp, Spinacia oleracea
  CDS (264)..(1814): coding for episilon-cyclase
gcacgagacaccacaaaaccattgaggagagagaaagtcaaccaaatttcacaccttccacctccctcttccatggccgcaacccaaacccagccaccttcaccgccgccgtcgacagcacactgaacttcaccactacaaacttaaaaaaaatcttggagaaatttgattccgtaaaaatggagtattattgtctcggagcttcgaaattcgcaacaatggcggtttctcctgcgcttaatcacgacaatttgaggaataaaatggttaaacaacgccagaatttccagacgttttgcttttggaggccgaattcttcgaacgttgtagtagaatgtagtagtcgtaggagtggaagtagtgttttgaggagtgcgaatagcgacagtagttgcgtaattgcgccagaggattttgcgaacgaagaagatttcatcaaagctggtggttccgagcttctttatgttcaaatgcagcagaataaagctatggattgttactccaaaatttccgataagctgcgtcaaatatcagatgccaatgaactgctggatatggtggttattggttgtggtccagctggtctagctttggctgcagaatcggctaaacttggattaaaagttggccttgttggtcctgatcttccttttacgaataactacggcgtttgggaagatgaattcagagcattgggacttggaggctgtatcgagcacgtttggcgtgataccattgtgtatattgatgatgacaatcctatatatattggtcgatcttatggaaaagtcagccggcaattacttcacaaggaactggtgcacaggtgtttggagtcaggtgtctcttatctgaatgcgaaagtggaaaatattatggaaggacctgatggacataggcttgttgcttgtgaacgtggtgtcactattccctgcaggcttgtaactgttgcatctggagcagcttcagggaaacttctggagtatgaagtgggtggtccaagggtttgtgtacaaacagcttatggtgtggaggtggaggtggaaaacagtccttatgatcccaatgtgatggtgttcatggactacagagactacactaaactgagcgttcaatctctggaggcaaagtatccaacattcttgtatgcaatgccgatatcaccaactaggatcttctttgaggagacttgcttggcttcagtagatgcaatgccctttgacctgctcaagaaaaagcttatgacaagattacaaactatgggtgttcgtatcaccaaaatatatgaagaggagtggtcttatatacctgttggtgggtccttaccaaatacagagcaaagaaaccttgcatttggtgctgctgcgagcatggtgcatccagccacaggttattcagtcgtgagatcactgtcagaagctccaaagtatgcttctgcaattgcaaacttgatcaagaatgacctgtcaaaaaatgcaatattgcgtcagaggagtgtggggaatatctcaatgcaagcctggaatactctttggccacaagaaaggaaacgtcagagagcattcttcctgttcggactatcacttatagtccagcttgatattgagggtatcaggacattcttccgcaccttcttccgagtgccaaaatggatgtgggagggattcctcggttctaatctctcttcagctgatctcatattgtttgccttttatatgttctttattgctccgaatgacttgagaatgggtcttataaggcatctactatctgatcctacaggggcgaccatgataagaacgtacataacactataaaagtaatatgaaatgctcactcctttgtacatcatgcaaaattggtacgaattgactggactatgcagtctaacttggtgtaaaaaaaacacaattaataaattttttgtaggtgcagcctctatacttgatattctcgattcagatataatattgtcagtattcttcgttaaagatcagttgtttctacaattccagaggctcctggaattggtgttacccttccatgtagctcattgataaatgttgagggtagaggctttttcttagatgcttgcttgcagcttgctcatggatatattcagttgttcagtagacacgttaacaactactacagtgggggcatcattgatctggaccgggagagctgagcatctatcacaggttagccagctcaactacgtaggtcaaccttgagccactcccaaacatttttgcagctgatggggttcaccctgtaaggtgagtttcttaccaactccaccaacttatgttggttttaaattgctactcgtctgttatgaagtagcaagctcgtgc

SEQ ID NO: 38 - protein, 517 aa, Spinacia oleracea
MVKQRQNFQTFCFWRPNSSNVVVECSSRRSGSSVLRSANSDSSCVIAPEDFANEEDFIKAGGSELLYVQMQQNKAMDCYSKISDKLRQISDANELLDMVVIGCGPAGLALAAESAKLGLKVGLVGPDLPFTNNYGVWEDEFRALGLGGCIEHVWRDTIVYIDDDNPIYIGRSYGKVSRQLLHKELVHRCLESGVSYLNAKVENIMEGPDGHRLVACERGVTIPCRLVTVASGAASGKLLEYEVGGPRVCVQTAYGVEVEVENSPYDPNVMVFMDYRDYTKLSVQSLEAKYPTFLYAMPISPTRIFFEETCLASVDAMPFDLLKKKLMTRLQTMGVRITKIYEEEWSYIPVGGSLPNTEQRNLAFGAAASMVHPATGYSVVRSLSEAPKYASAIANLIKNDLSKNAILRQRSVGNISMQAWNTLWPQERKRQRAFFLFGLSLIVQLDIEGIRTFFRTFFRVPKWMWEGFLGSNLSSADLILFAFYMFFIAPNDLRMGLIRHLLSDPTGATMIRTYITL

SEQ ID NO: 39 - DNA, 1378 bp, Solanum tuberosum
  CDS (2)..(1147): coding for episilon-cyclase (partial)
tagcggnnnnnaggatgagttcaaagatcttggtcttcaagcctgcattgaacatgtttggcgggataccattgtatatcttgatgatgatgatcctattcttattggccgtgcctatggaagagttagtcgccatttactgcacgaggagttactcaaaaggtgtgtggaggcaggtgttttgtatctaaactcgaaagtggataggattgttgaggccacaaatggccacagtcttgtagagtgcgagggtgatgttgtgattccctgcaggtttgtgactgttgcatcgggagcagcctcggggaaattcttgcagtatgagttgggaggtcctagagtttctgttcaaacagcttatggagtggaagttgaggtcgataacaatccatttgacccgagcctgatggttttcatggattatagagactatgtcagacacgacgctcaatctttagaagctaaatatccaacatttctctatgccatgcccatgtctccaacacgagtctttttcgaggaaacttgtttggcttcaaaagatgcaatgccattcgatctgttaaagaaaaaattgatgttacgattgaacaccctcggtgtaagaattaaagaaatttatgaggaggaatggtcttacataccagttggaggatctttgccaaatacagaacaaaaaacacttgcatttggtgctgctgctagcatggttcatccagccacaggttattcagtcgtcagatcactgtctgaagctccaaaatgcgccttcgtgcttgcaaatatattacgacaaaatcatagcaagaatatgcttactagttcaagtaccccgagtatttcaactcaagcttggaacactctttggccacaagaacgaaaacgacaaagatcgtttttcctatttggactggctctgatattgcagctggatattgaggggataaggtcatttttccgcgcgttcttccgtgtgccaaaatggatgtggcagggatttcttggttcaagtctttcttnagcagacctcatgttatttgccttctacatgtttattattgcaccaaatgacatgagaagaggcttaatcagacatcttttatctgatcctactggtgcaacattgataagaacttatcttacattttagagtaaattcctcctacaatagttgttgaannagaggcctcattacttcagattcataacagaaatcgcggtctctcgaggccttgtatataacattttcactaggttaatattgcttgaataagttgcacagtttcagtttttgtatctgcttcttttttgtccaagatcatgtattganccaatttatatacattgccagtatatataaattttataaaaaaaaaaa

SEQ ID NO: 40 - protein, 382 aa, Solanum tuberosum
  MISC_FEATURE (2)..(4), (340): each occurrence of Xaa is any amino acid
SXXXDEFKDLGLQACIEHVWRDTIVYLDDDDPILIGRAYGRVSRHLLHEELLKRCVEAGVLYLNSKVDRIVEATNGHSLVECEGDVVIPCRFVTVASGAASGKFLQYELGGPRVSVQTAYGVEVEVDNNPFDPSLMVFMDYRDYVRHDAQSLEAKYPTFLYAMPMSPTRVFFEETCLASKDAMPFDLLKKKLMLRLNTLGVRIKEIYEEEWSYIPVGGSLPNTEQKTLAFGAAASMVHPATGYSVVRSLSEAPKCAFVLANILRQNHSKNMLTSSSTPSISTQAWNTLWPQERKRQRSFFLFGLALILQLDIEGIRSFFRAFFRVPKWMWQGFLGSSLSXADLMLFAFYMFIIAPNDMRRGLIRHLLSDPTGATLIRTYLTF

SEQ ID NO: 41 - DNA, 497 bp, Daucus carota
  CDS (1)..(495): coding for episilon-cyclase  (partial)
tatggtgtttgggtggatgaatttatagatcttggacttgaagggtgtattgagcatgtttggcgggatactattgtatatcttgatgatggtgatcctattatgattggccgtgcttacggaagagttagtcgccatttgcttcatgaagaattgcttaaaaggtgtgtcgagtcaggtgtttcgtatcttagctcaaaagttgaaaagattattgaagctggagatggccacagcctggttgagtgtgaaaataatattgtcattccatgcaggcttgctactgttgcatctggagcagcttctgggaaacttttgcagtatgaggttgggggtcccagagtttctgtccaaacagcttatggtgtcgaggttgaggtggaaaacaatccatatgatcccagtctaatggttttcatggattacagagattataccaaacaaaaagttccaggcatggaggcagaatatccaactttcctgtatgccatgccatt

SEQ ID NO: 42 - protein, 165 aa, Daucus carota
YGVWVDEFIDLGLEGCIEHVWRDTIVYLDDGDPIMIGRAYGRVSRHLLHEELLKRCVESGVSYLSSKVEKIIEAGDGHSLVECENNIVIPCRLATVASGAASGKLLQYEVGGPRVSVQTAYGVEVEVENNPYDPSLMVFMDYRDYTKQKVPGMEAEYPTFLYAMP

SEQ ID NO: 43 - DNA, 605 bp, Daucus carota
  CDS (3)..(605): coding for episilon-cyclase     (partial)
tcattggccgtgcttatggaagattagtcgccatttgcttcatgaagaattgcttaaaaggtgtgtcgagtcaggtgtttcgtatcttagctcaaaagttgaaaagattattgaagctggagatggccacagcctggttgagtgtgaaaataatattgtcattccatgcaggcttgctactgttgcatctggagcagcttctgggaaacttttgcagtatggggttgggggtcccagagtttctgtccaaacagcttatggtgtcgaggttgaggtggaaaccaatcccatatgatcccagtctaatggttttcatggattacagagattataccaaacaaaaagttccaggcatggaggcagaatatccaacatttctttatgtgatgcccatgtccccaacaaggattttctttgaggagacatgtttggcttcaaaagatgcgatgccattcgatctactgaagaaaaaactcatgtcaagattacagacgatgggaattcgagttgccaagacatatgaagaggaatggtcttatatacctgttgggggatctttacctaatactgagcaaaagaatctcgcctttggtgctgccgctaga

SEQ ID NO: 44 - protein, 201 aa, Daucus carota
IGRAYGRLVAICFMKNCLKGVSSQVFRILAQKLKRLLKLEMATAWLSVKIILSFHAGLLLLHLEQLLGNFCSMGLGVPEFLSKQLMVSRLRWKPIPYDPSLMVFMDYRDYTKQKVPGMEAEYPTFLYVMPMSPTRIFFEETCLASKDAMPFDLLKKKLMSRLQTMGIRVAKTYEEEWSYIPVGGSLPNTEQKNLAFGAAAR

SEQ ID NO: 45 - DNA, 1697 bp, Lycopersicon esculentum
  CDS (6)..(1583): coding for epsilon-cyclase
ttgaaatggagtgtgttggagttcaaaatgttggagcaatggcagttttaacgcgtccgagattgaaccgttggtcgggaggagagttatgccaagaaaaaagcatctttttggcgtatgagcagtatgaaagtaaatgtaatagcagtagtggtagtgacagttgtgtagttgataaagaagattttgctgatgaagaagattatataaaagccggtggttcgcaacttgtatttgttcaaatgcagcagaaaaaagatatggatcagcagtctaagctttctgatgagttacgacaaatatctgctggacaaaccgtactggatttagtggtaatcggctgtggtcctgctggtcttgctcttgccgcggagtcagctaaattggggttgaacgtggggctcgttgggcctgatcttcctttcacaaacaactatggtgtatgggaggacgagttcaaagatcttggtcttcaagcctgcattgaacatgtttggcgggataccattgtatatcttgatgatgatgaacctattcttattggccgtgcctatggaagagttagtcgccattttctgcacgaggagttactcaaaaggtgtgtggaggcaggtgttttgtatctaaactcgaaagtggataggattgttgaggccacaaatggccagagtcttgtagagtgcgaaggtgatgttgtgattccctgcaggtttgtgactgttgcatcgggggcagcctcggggaaattcttgcagtatgagttgggaagtcctagagtttctgttcaaacagcttatggagtggaagttgaggttgataacaatccatttgacccgagcctgatggttttcatggattatagagattatctcagacacgacgctcaatctttagaagctaaatatccaacatttctttatgccatgcccatgtctccaacacgagtctttttcgaggaaacttgtttggcttcaaaagatgcaatgccattcgatctgttaaagaaaaaactgatgctacgattgaacacccttggtgtaagaattaaagaaatttacgaggaggaatggtcttacataccggttggtggatctttgccaaatacagaacaaaaaacacttgcatttggtgctgctgctagcatggttcatccagccacaggttattcagtcgtcagatcactttctgaagctccaaaatgcgcctctgtacttgcaaatatattacgacaacattatagcaagaacatgcttaccagttcaagtatcccgagtatatcaactcaagcttggaacactctttggccacaagaacgaaaacgacaaagatcgtttttcctatttggactggctctgatattgcagctggatattgaggggataaggtcatttttccgcgcattcttccgtgtgccaaaatggatgtggcagggatttcttggttcaagtctttcttcagcagacctcatgttatttgccttctacatgtttattattgcaccaaatgacatgagaaaaggcttgatcagacatcttttatctgatcctactggtgcaacattgataagaacttatcttacattttagagtaaactcctcctacaataattgttgaatcagaggcctcattacttcagattcataacagaaatcgcggtctctcgaggcattgtatataacattttcactagcttaata

SEQ ID NO: 46 - protein, 526 aa, Lycopersicon esculentum
MECVGVQNVGAMAVLTRPRLNRWSGGELCQEKSIFLAYEQYESKCNSSSGSDSCVVDKEDFADEEDYIKAGGSQLVFVQMQQKKDMDQQSKLSDELRQISAGQTVLDLVVIGCGPAGLALAAESAKLGLNVGLVGPDLPFTNNYGVWEDEFKDLGLQACIEHVWRDTIVYLDDDEPILIGRAYGRVSRHFLHEELLKRCVEAGVLYLNSKVDRIVEATNGQSLVECEGDVVIPCRFVTVASGAASGKFLQYELGSPRVSVQTAYGVEVEVDNNPFDPSLMVFMDYRDYLRHDAQSLEAKYPTFLYAMPMSPTRVFFEETCLASKDAMPFDLLKKKLMLRLNTLGVRIKEIYEEEWSYIPVGGSLPNTEQKTLAFGAAASMVHPATGYSVVRSLSEAPKCASVLANILRQHYSKNMLTSSSIPSISTQAWNTLWPQERKRQRSFFLFGLALILQLDIEGIRSFFRAFFRVPKWMWQGFLGSSLSSADLMLFAFYMFIIAPNDMRKGLIRHLLSDPTGATLIRTYLTF

SEQ ID NO: 47 - DNA, 510 bp, Tagetes erecta
  misc_feature (1)..(510): coding for epsilon-cyclase specific probe
ggcacgaggcaaagcaaaggttgtttgttgttgttgttgagagacactccaatccaaacagatacaaggcgtgactggatatttctctctcgttcctaacaacagcaacgaagaagaaaaagaatcattactaacaatcaatgagtatgagagctggacacatgacggcaacaatggcggcttttacatgccctaggtttatgactagcatcagatacacgaagcaaattaagtgcaacgctgctaaaagccagctagtcgttaaacaagagattgaggaggaagaagattatgtgaaagccggtggatcggagctgctttttgttcaaatgcaacagaataagtccatggatgcacagtctagcctatcccaaaagctcccaagggtaccaataggaggaggaggagacagtaactgtatactggatttggttgtaattggttgtggtcctgctggccttgctcttgctggagaatcagccaagctaggcttgaatgtcgcacttatcg

SEQ ID NO: 48 - DNA, 20 bp, Artificial sequence
  Description of the artificial sequence oligonucleotide primer
ggcacgaggcaaagcaaagg

SEQ ID NO: 49 - DNA, 21 bp, Artificial sequence
  Description of the artificial sequence oligonucleotide primer
cgataagtgcgacattcaagc

SEQ ID NO: 50 - DNA, 734 bp, Tagetes erecta
  misc_feature (1)..(734): fragment of epsilon cyclase gene obtain by iPCR comprising part of  promoter region
ctaacaatcaatgagtagagagctggacacatgacggcaacaatggcggcttttacatgccctaggtttatgactagcatcagatacacgaagcaaattaagtgcaacgctgctaaaagccagctagtcgttaaacaagagattgaggaggaagaagattatgtgaaagccggtggatcggagctgctttttgttcaaatgcaacagaataagtccatggatgcacagtctagcctatcccaaaaggtcactccagacttaattgcttataaataaataaatatgttttttaggaataatgatatttagatagattagctatcacctgtgctgtggtgtgcagctcccaagggtcttaccgatagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggscaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaaggttgtttgttgttgttgttgagagacactccaatccaaacagatacaaggcg

SEQ ID NO: 51 - DNA, 280 bp, Tagetes erecta
  misc_feature (1)..(280): fragment of epsilon cyclase gene obtain by TAIL-PCR comprising part of  promoter region
gtcgagtatggagttcaattaaaataaagagaagaraaagattaagagggtgatggggatattaaagacggccaatrtagtgatgccacgtaagaaaaaggtaagtgaaaacatacaacgtggctttaaaagatggcttggctgctaatcaactcaactcaactcatatcctatccattcaaattcaattcaattctattgaatgcaaagcaaagcaaagcaaaggttgtttgttgttgttgttgagagacactccaatccaaacagatacaaggcgtga

SEQ ID NO: 52 - DNA, 23 bp, Artificial sequence
  Description of the artificial sequence oligonucleotide primer
cgccttgtatctgtttggattgg

SEQ ID NO: 53 - DNA, 24 bp, Artificial sequence
  Description of the artificial sequence oligonucleotide primer
ctaacaatcaatgagtatgagagc

SEQ ID NO: 54 - DNA, 26 bp, Artificial sequence
  Description of the artificial sequence oligonucleotide primer
agagcaaggccagcaggaccacaacc

SEQ ID NO: 55 - DNA, 26 bp, Artificial sequence
  Description of the artificial sequence oligonucleotide primer
ccttgggagcttttgggataggctag

SEQ ID NO: 56 - DNA, 26 bp, Artificial sequence
  Description of the artificial sequence oligonucleotide primer
tcacgccttgtatctgtttggattgg

SEQ ID NO: 57 - DNA, 15 bp, Artificial sequence
  Description of the artificial sequence oligonucleotide primer
gtcgagtatggagtt

SEQ ID NO: 58 - DNA, 734 bp, Tagetes erecta; identical to SEQ ID NO: 50
  misc_feature (1)..(734): coding for epsilon-cyclase genomic iPCR-fragment

SEQ ID NO: 59 - DNA, 25 bp, Artificial sequence
  Description of the artificial sequence oligonucleotide primer
ctcgagagtaaaatcgttagttatg

SEQ ID NO: 60 - DNA, 29 bp, Artificial sequence
  Description of the artificial sequence oligonucleotide primer
ccatggccattgattgttagtaatgattc

SEQ ID NO: 61 - DNA, 29 bp, Artificial sequence
  Description of the artificial sequence oligonucleotide primer
ccatggtaatttgcttcgtgtatctgatg

SEQ ID NO: 62 - DNA, 26 bp, Artificial sequence
  Description of the artificial sequence oligonucleotide primer
ccatggcgctagcagcgacagtaatg

SEQ ID NO: 63 - DNA, 23 bp, Artificial sequence
  Description of the artificial sequence oligonucleotide primer
gatatccggtgtgagggaactag

SEQ ID NO: 64 - DNA, 22 bp, Artificial sequence
  Description of the artificial sequence oligonucleotide primer
gcaagctcgacagctacaaacc

SEQ ID NO: 65 - DNA, 24 bp, Artificial sequence
  Description of the artificial sequence oligonucleotide primer
gaagcatgcagctagcagcgacag

SEQ ID NO: 66 - DNA, 1795 bp, Artificial sequence
  Description of the artificial sequence coding for ketolase - 35S terminator construct
ccatggcgctagcagcgacagtaatgttggagcagcttaccggaagcgctgaggcactcaaggagaaggagaaggaggttgcaggcagctctgacgtgttgcgtacatgggcgacccagtactcgcttccgtcagaggagtcagacgcggcccgcccgggactgaagaatgcctacaagccaccaccttccgacacaaagggcatcacaatggcgctagctgtcatcggctcctgggccgcagtgttcctccacgccatttttcaaatcaagcttccgacctccttggaccagctgcactggctgcccgtgtcagatgccacagctcagctggttagcggcagcagcagcctgctgcacatcgtcgtagtattctttgtcctggagttcctgtacacaggcctttttatcaccacgcatgatgctatgcatggcaccatcgccatgagaaacaggcagcttaatgacttcttgggcagagtatgcatctccttgtacgcctggtttgattacaacatgctgcaccgcaagcattgggagcaccacaaccacactggcgaggtgggcaaggaccctgacttccacaggggaaaccctggcattgtgccctggtttgccagcttcatgtccagctacatgtcgatgtggcagtttgcgcgcctcgcatggtggacggtggtcatgcagctgctgggtgcgccaatggcgaacctgctggtgttcatggcggccgcgcccatcctgtccgccttccgcttgttctactttggcacgtacatgccccacaagcctgagcctggcgccgcgtcaggctcttcaccagccgtcatgaactggtggaagtcgcgcactagccaggcgtccgacctggtcagctttctgacctgctaccacttcgacctgcactgggagcaccaccgctggccctttgccccctggtgggagctgcccaactgccgccgcctgtctggccgaggtctggttcctgcctagctggacacactgcagtgggccctgctgccagctgggcatgcctgcaggtcgacggatccccgggaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggatatc

SEQ ID NO: 67 - DNA, 28 bp, Artificial sequence
  Description of the artificial sequence oligonucleotide primer
gagctcactcactgatttccattgcttg

SEQ ID NO: 68 - DNA, 37 bp, Artificial sequence
  Description of the artificial sequence oligonucleotide primer
cgccgttaagtcgatgtccgttgatttaaacagtgtc

SEQ ID NO: 69 - DNA, 34 bp, Artificial sequence
  Description of the artificial sequence oligonucleotide primer
atcaacggacatcgacttaacggcgtttgtaaac

SEQ ID NO: 70 - DNA, 25 bp, Artificial sequence
  Description of the artificial sequence oligonucleotide primer
taagctttttgttgaagagatttgg

SEQ ID NO: 71 - DNA, 28 bp, Artificial sequence
  Description of the artificial sequence oligonucleotide primer
gtcgactacgtaagtttctgcttctacc

SEQ ID NO: 72 - DNA, 26 bp, Artificial sequence
  Description of the artificial sequence oligonucleotide primer
ggatccggtgatacctgcacatcaac

SEQ ID NO: 73 - DNA, 28 bp, Artificial sequence
  Description of the artificial sequence oligonucleotide primer
aagcttaccgatagtaaaatcgttagtt

SEQ ID NO: 74 - DNA, 31 bp, Artificial sequence
  Description of the artificial sequence oligonucleotide primer
ctcgagcttaccgatagtaaaatcgttagtt

SEQ ID NO: 75 - DNA, 28 bp, Artificial sequence
  Description of the artificial sequence oligonucleotide primer
gtcgacaacaacaacaaacaacctttgc

SEQ ID NO: 76 - DNA, 28 bp, Artificial sequence
  Description of the artificial sequence oligonucleotide primer
ggatccaacaacaacaaacaacctttgc

SEQ ID NO: 77 - DNA, 777 bp, Arabidopsis thaliana
  promoter (1)..(777): modified version of the AP3 promoter
gagctcactcactgatttccattgcttgaaaattgatgatgaactaagatcaatccatgttagtttcaaaacaacagtaactgtggccaacttagttttgaaacaacactaactggtcgaagcaaaaagaaaaaagagtttcatcatatatctgatttgatggactgtttggagttaggaccaaacattatctacaaacaaagacttttctcctaacttgtgattccttcttaaaccctaggggtaatattctattttccaaggatctttagttaaaggcaaatccgggaaattattgtaatcatttggggaaacatataaaagatttgagttagatggaagtgacgattaatccaaacatatatatctctttcttcttatttcccaaattaacagacaaaagtagaatattggcttttaacaccaatataaaaacttgcttcacacctaaacacttttgtttactttagggtaagtgcaaaaagccaaccaaatccacctgcactgatttgacgtttacaaacgccgttaagtcgatgtccgttgatttaaacagtgtcttgtaattaaaaaaatcagtttacataaatggaaaatttatcacttagttttcatcaacttctgaacttacctttcatggattaggcaatactttccatttttagtaactcaagtggaccctttacttcttcaactccatctctctctttctatttcacttctttcttctcattatatctcttgtcctctccaccaaatctcttcaacaaaaagctt

SEQ ID NO: 78 - DNA, 212 bp, Solanum tuberosum
  intron (1)..(212): PIV2 intron of ST-LS1 gene
gtcgactacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccggatcc

SEQ ID NO: 79 - DNA, 358 bp, Tagetes erecta
  misc_feature (1)..(358): coding for sense-strand of epsilon cyclase promoter directed dsRNA
aagcttaccgatagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggccaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaagcaaaggttgtttgttgttgttgtcgac

SEQ ID NO: 80 - DNA, 361 bp, Tagetes erecta
  misc_feature (1)..(361): coding for antisense-strand of epsilon cyclase promoter directed dsRNA
ctcgagcttaccgatagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggccaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaagcaaaggttgtttgttgttgttggatcc

SEQ ID NO: 81 - DNA, 1537 bp, Cucumis sativus
  promoter (1)..(1537): promoter of chromoplast-specific carotenoid-associated protein (CHRC)
gagctctacaaattagggttactttattcattttcatccattctctttattgttaaattttgtacatttattcaataatattatatgtttattacaaattctcactttcttattcatacctattcactcaagcctttaccatcttccttttctatttcaatactatttctacttcatttttcacgtttttaacatctttctttatttcttgtccacttcgtttagggatgcctaatgtcccaaatttcatctctcgtagtaacacaaaaccaatgtaatgctacttctctctacatttttaatacaaataaagtgaaacaaaatatctataaataaacaaatatatatattttgttagacgctgtctcaacccatcaattaaaaaattttgttatatttctactttacctactaaatttgtttctcatatttaccttttaacccccacaaaaaaaaattataaaaaagaaagaaaaaagctaaaccctatttaaatagctaactataagatcttaaaattatcctcatcagtgtatagtttaattggttattaacttataacattatatatctatgacatatactctctcctagctatttctcacattttttaacttaagaaaatagtcataacatagtctaaaattcaaacatccacatgctctaatttgattaacaaaaagttagaaatatttatttaaataaaaaagactaataaatatataaaatgaatgttcatacgcagacccatttagagatgagtatgctttcacatgctgagattattttcaaaactaaggttgtagcaatattaaatcaataaaattattataaataacaaaattaacctgctcgtgtttgctgtatatgggaggctacaaaataaattaaactaaagatgattatgttttagacattttttctatctgtattagtttatacatattaattcaggagctgcacaacccaattctattttcgttccttggtggctgggtttctcacaaggttcaatagtcaatattaggttttattggacttttaatagtatcaaacaaatctatgtgtgaacttaaaaattgtattaaatatttagggtaacctgttgccgtttttagaataatgtttcttcttaatacacgaaagcgtattgtgtattcattcatttggcgcctcacatgcttcggttggctcgctttagtctctgccttctttgtatattgtactccccctcttcctatgccacgtgttctgagcttaacaagccacgttgcgtgccattgccaaacaagtcattttaacttcacaaggtccgatttgacctccaaaacaacgacaagtttccgaacagtcgcgaagatcaagggtataatcgtctttttgaattctatttctctttatttaatagtccctctcgtgtgatagtttttaaaagatttttaaaacgtagctgctgtttaagtaaatcccagtccttcagtttgtgcttttgtgtgttttgtttctctgatttacggaatttggaaataataagctt

SEQ ID NO: 82 - DNA, 22 bp, Artificial sequence
  Description of the artificial sequence oligonucleotide primer
gagctctacaaattagggttac

SEQ ID NO: 83 - DNA, 23 bp, Artificial sequence
  Description of the artificial sequence oligonucleotide primer
aagcttattatttccaaattccg